SEQUENCE LISTING (89 sequences)

SEQ ID NO: 1 - protein, 155 aa, Escherichia coli
MIKRVLVVSMVGLSLVGCVNNDTLSGDVYTASEAKQVQNVSYGTIVNVRPVQIQGGDDSNVIGAIGGAVLGGFLGNTVGGGTGRSLATAAGAVAGGVAGQGVQSAMNKTQGVELEIRKDDGNTIMVVQKQGNTRFSPGQRVVLASNGSQVTVSPR

SEQ ID NO: 2 - protein, 155 aa, Serratia sp.
MMKYFLVITLASITLVGCANTSTLSGDVYSASDAKQVQTVTYGTIISTRPVQIQAGEENNIIGALGGAVLGGFLGNTVGGGTGRSLATAAGAVAGGVAGQSVEGAINRVQGVQLEIRKDDGNTIMVVQKQGTTKFYAGQRIAMVSDGRSVTVSPR

SEQ ID NO: 3 - protein, 155 aa, Erwinia sp.
MMMRLLIVALTGVTLAGCVNDSSLSGDVYSASEAKQIQNVTYGTLVSVRPVQIQGDDNTNVIGAIGGAVLGGFLGNTVGGGTGRSLATAAGAVAGGVAGQGVQSSMNKSQGVELEIRRDDGSTIAVVQKQAASRYSVGQRVAMTSNGREVTVSPR

SEQ ID NO: 4 - protein, 155 aa, Edwardsiella tarda
MMKRLIIVAIASATLAGCANTSTLSGDVYSANQAKQVQQVSYGTIVSIEPVRIQAGNEANMIGTIGGAVIGGLLGNTIGGGTGRSLATAAGAVAGGVAGNSIEGAVNRTDGVQLVIKMDSGKTIAVVQKADKQAFRVNQRVMLLSNGSSVNVSPM

SEQ ID NO: 5 - protein, 155 aa, Serratia odorifera
MIKRLIVVAFAGLSLVGCANDSMSGDVYSASQAKQVQQASYGTLVSVRPVKISGEGGSNMLGTIGGAVIGGFLGNTIGGGTGRSLATAAGAVAGGVAGDQIGEAAGRTSGVELEIRPDTKGDNIIVVQKAGTTKFSPGQRVRMARSGSTITVSPL

SEQ ID NO: 6 - protein, 155 aa, Proteus penneri
MFKHLLIGLFTMTVLTGCVNTSSLSGDTYTASQAKQAQNITYGTVVSVRSVNIQAGSDENILGAIGGAVLGGMLGNTVGGGTGRNLATAAGAIAGGMAGQQAQGALNTTKGVQIEVRLDSGRTVAIVQKADNTVYSKGQRVAVIGNGNNLTVSPR

SEQ ID NO: 7 - protein, 157 aa, Xenorhabdus nematophilus
MFKRFLVGAVVVTTLSGCADMGALSSDTYSMDQAKQAQTVTYGTILSVRPVTIKGNQAGDPNVLGLIGGAVLGGLLGNTVGGGSGQRLATAAGAIAGGMAGQRIEGAIDQTKGVELEIRTDSGKNIIVVQKLDQVVFNRGQRVRIANSGNSLTVSPR

SEQ ID NO: 8 - protein, 165 aa, Gilliamella bombicola
MKKIVTLAILATILSGCSNSDLYSGDVYTADQAKQVQQVSYGTVVSVRPVKIQANASSDGKSENVAGNLGGAVLGGVLGNTIGGGTGRVLAVATGAIGGAILGDTIQDKTSQASAVELEIKQENGSNIVIVQKGSAKDFFAGQAVRLVTNGKKISASPRYNNQSK

SEQ ID NO: 9 - protein, 155 aa, Halomonas anticariensis
MKRLLPILAIGLLTLAGCANTAPYSGDVYRGSQAQTGQTVSYGTIVALRQVQIQADNPQGNVVGGLGGAVIGGLLGNQIGGGSGRTLATAAGAIGGSVAGSKIEDRANRTTAWEMEIRRDRGDNVIVVQKADRAFQVGQRVRLIGSGANLSVAPY

SEQ ID NO: 10 - protein, 157 aa, Oceanisphaera profunda
MKRNTYSAIALAAALGLAGCANPDVYSGDVYGSRAAKQAQTVTYATVTSVRPVLIQAGDENPNVIGTLGGGAIGGILGHQVGGGTGQVLATAVGALAGTMVGSRAEDKLSQVKAVELEVRTDSGENLIVVQKADRTWQPGQRVRLVGSGSNLSVAPN

SEQ ID NO: 11 - protein, 154 aa, Aeromonas molluscorum
MLQRLILIALLAAAVTGCANSDVYSGDVYTKDRAKQVQTVSYGTIVSTRPVKIQAEENSILGTIGGGVVGGLLGSTIGGGTGRSLAAAGGAIAGAAAGKAAGDKLNQVDGVELEIKQDNGSSIVVVQKADPSFQPGAKVRLTQGNGSVNAAVVP

SEQ ID NO: 12 - protein, 153 aa, Haemophilus pittmaniae
MKKMTVALALLVALGVTGCANTDVFSGDVYTADQAKEARSISYGTILSVRPVKIQADNPGVIGTIGGGALGGIAGSTVGGGTGQALATAVGAIAGAVVGSKVEEKASQVNAAELVIRKDNGKQIVVVQKADPNFVAGRRVRIVGGSSLNVSVL

SEQ ID NO: 13 - protein, 154 aa, Simonsiella muelleri
MKKNIILTTLMAALAVTGCANKSIYSGNVYTSSQAKETRAISYGTIVSVREVQIQAPSSGVIGTTAGGALGGIAGSSMGGGRGSDLLGVLVGTVGAIVGNQIEQGMSKVASLEMVIRKDDGAEIVVVQKKEAGLTVGKRVRLVGSASDVNVSPL

SEQ ID NO: 14 - protein, 158 aa, Shewanella halifaxensis
MTIKTNFVVLLTMSIATIAGCTTPTPYGDAYAASETRTIQQVYYGTIVKAEPVTIAASDKTNIIAMIAGAAIGGILGSEVGGGAGSDIAAIGGSVAGGYAGSEIADAAGERNGVNLTIRLEDGRIISIVQEVNPSMIFQQGQAVQVNVDGNTARVVPR

SEQ ID NO: 15 - protein, 163 aa, Pseudogulbenkiania sp.
MIHRKEMNMLKSALRTLVAAALVTGTLAGCATSDSAAVYSKGQMRQAQNVEFGTVVSVQNVLMEGNNNELLTLGGAALGGIAGSTIGQGRGAAAGTIVGALAGGFGTQAAQRNLGTKNALEITVKLDRSGRMLSIVQEADIPFAPGQRVKVLTGGGNDRVAPL

SEQ ID NO: 16 - protein, 154 aa, Klebsiella variicola
MLKKMCLIAFTLVIAGCTNTSHLSGDVHSSATAGRVQQVFYGQLVSVRAVQIQAGDNSNALGAVAGGVIGGVAGSAVGGGSGQRLATATGAVAGGLAGQAVQARMNLVQGVELEIRLDDGRTFMVVQRQGPTRFSVGQRVAISSVGGQTTVSPR

SEQ ID NO: 17 - protein, 156 aa, Klebsiella pneumoniae
MFTRSVLMLAGLLALAGCTEKPVGSAHHASAALSGQVMALSHGTITGVRYITIQNDTGSSAAGTVGGALIGGAAGNAIGGGSGRLLATAGGTVAGAMAGQSIQRGMARHNAAELFIRPDNGQEFVTVQPAREGEFVVGQRVNITSHRGQVTISPAQ

SEQ ID NO: 18 - protein, 165 aa, Bordetella avium
MSTTISPSVGRIGRGIAVVTLAAGMLVLAGCANHSASSSVYTYGQAQREQIVRTGTVTGVRPITIQTDKSSGVGALAGGALGGVAGNAIGGGTGRTIATVGGAILGALAGNVAENQVGKTSGYEITVRLDNGETRVVAQEADNPVSVGQRVQVISGAGPTRVVPY

SEQ ID NO: 19 - protein, 166 aa, Pusillimonas sp.
MISTSFRHSVARPARLLALTALVGSLSMLAACANDNASSSVYSYGQAQREQIVRTGTVEAVRNVTIQKDSTSGAGMIAGGALGGVAASTIGGGTGQVLATIGGGILGAMAGNAVENQVGKTKGLEITVRLDNGETRVIAQAADVPVGVGQRVRLISGNGPTRVVPM

SEQ ID NO: 20 - protein, 165 aa, Achromobacter arsenitoxydans
MHKHQQAVKRSPLTTIGAGAILGLAVILAGCANPSASSRVYTFDQAQRAQTVTLGYVVGVRPVTIQASQTSGLGVMAGGALGAVTGSAIGGGSGRRITTVGGAVGGAMAGNAIENASARRAGLELTVRLDSGDLRVITQEADVPLSTGQRVQIVTQGGVSRVVGI

SEQ ID NO: 21 - protein, 158 aa, Paraburkholderia phytofirmans
MRTTSRLVVAALIAGSLAMSGCAYNSSSADVYTASQAQREETVRMGTVDSVRAVKISSNNGQPSGLGAIGGGALGAVAGSAIGGGRGSIVTGIIGGLAGAVAGNAVENGAAVHDGLEITVRLDNGDMRAITQSATGEIFRAGERVRLLSSGGVTRVTH

SEQ ID NO: 22 - protein, 156 aa, Burkholderia cepacia
MSSPKVLTFVMLTAAATLTGCITPPGSADVYSSGQAQREQTVRMGTVESVRATRIQGSDGGSVLGSLGGGALGAVAGSAIGGGRGSVLTTIAGGIAGAVAGNAVGQGLSSANGVEITVRLDNGDLRSIAQAATPETFRAGDRVRLLSSAGVTRVTH

SEQ ID NO: 23 - protein, 148 aa, Janthinobacterium agaricidamnosum
MKALITIALISTLAGCAVAPNSNTVYTTRQAQSEQSVRLATVESVRQVTIDKGSSGTGVLAGAALGGLAGSTVGGGKGSIATSIIGAIAGGAVGQNVEANVNNKPGFEITVRLDNGELRSIVQDADEAFRPGERVRLLSDGRKTRVTH

SEQ ID NO: 24 - protein, 154 aa, Sedimenticola thiotaurini
MQYRFFIILIILTTITACTTSGLSGTTYSRTDARKVQTVKYGTIESVTPVVIEGRTDGIVGTGSGAVVGGIAGSTVGGGKGRTIAAVVGAVAGGLAGQAAEKKLTTKQGQELTIRLEATGDIISVVQEVENEQFFRPGDRIRLLQYGGNTRVAY

SEQ ID NO: 25 - protein, 154 aa, Pseudomonas fulva
MLKPGFFAALFAAATLTLGGCASSLTGDSYSRDEARAVQTVRMGTIESLRPVKIEGTKTPIGGGAGAVIGGVAGSGVGGGRGSAVAAVIGAVAGGLLGAAAEEGITRTQGVEITVREDDGSMRAYVQAVEENQIFRVGDRVRIMTVNGTSRVTH

SEQ ID NO: 26 - protein, 155 aa, Pseudomonas alcaligenes
MQRFPTLLLTASLSTVLLLSGCASNLNGSTYSRADARAVQTVRLGTIESLRPVQIEGTRTPIGAIAGAAVGGIAGSGVGGGRGSSIATVLGGVAGGMAGAAAEEGLTRSQGVEITVREDDGNLRAYVQEVDPAQQFRVGDRVRIASVNGASRVTL

SEQ ID NO: 27 - protein, 150 aa, Methylovorus glucosetrophus
MNTVKLVGIAVMLAMLGACASSNSGSVYSRDEARKVQTVKMGVVESVRTVKLEGTKSPVGTAGGAVVGGVAGSTMGGGKGQAIATVLGAIVGGLAGSAAEEGLTRKDGLEITVKLDSGTMIAVVQEADDQFQPGERVRILESGGTTRVSH

SEQ ID NO: 28 - protein, 157 aa, Thauera sp.
MVNHKGLKLRAAAGAIVAVLVLGGCAQGLGGGTYSRTEARRAMTVQFGTVESVRGVQLEGTKSPVGTVAGAAVGGIAGSTIGGGRGQAVATVIGAVAGGVAGSALEEGMTRTPGVEVTVRLDNGQFLAVVQADEGERLRPGERVRVLRDGGTTRVTR

SEQ ID NO: 29 - protein, 113 aa, Escherichia coli
MKITLSKRIGLLAILLPCALALSTTVHAETNKLVIESGDSAQSRQHAAMEKEQWNDTRNLRQKVNKRTEKEWDKADAAFDNRDKCEQSANINAYWEPNTLRCLDRRTGRVITP

SEQ ID NO: 30 - protein, 246 aa, Escherichia coli
MKFKVIALAALMGISGMAAQANELPDGPHIVTSGTASVDAVPDIATLAIEVNVAAKDAATAKKQADERVAQYISFLELNQIAKKDISSANLRTQPDYDYQDGKSILKGYRAVRTVEVTLRQLDKLNSLLDGALKAGLNEIRSVSLGVAQPDAYKDKARKAAIDNAIHQAQELANGFHRKLGPVYSVRYHVSNYQPSPMVRMMKADAAPVSAQETYEQAAIQFDDQVDVVFQLEPVDQQPAKTPAAQ

SEQ ID NO: 31 - protein, 810 aa, Escherichia coli
MAMKKLLIASLLFSSATVYGAEGFVVKDIHFEGLQRVAVGAALLSMPVRTGDTVNDEDISNTIRALFATGNFEDVRVLRDGDTLLVQVKERPTIASITFSGNKSVKDDMLKQNLEASGVRVGESLDRTTIADIEKGLEDFYYSVGKYSASVKAVVTPLPRNRVDLKLVFQEGVSAEIQQINIVGNHAFTTDELISHFQLRDEVPWWNVVGDRKYQKQKLAGDLETLRSYYLDRGYARFNIDSTQVSLTPDKKGIYVTVNITEGDQYKLSGVEVSGNLAGHSAEIEQLTKIEPGELYNGTKVTKMEDDIKKLLGRYGYAYPRVQSMPEINDADKTVKLRVNVDAGNRFYVRKIRFEGNDTSKDAVLRREMRQMEGAWLGSDLVDQGKERLNRLGFFETVDTDTQRVPGSPDQVDVVYKVKERNTGSFNFGIGYGTESGVSFQAGVQQDNWLGTGYAVGINGTKNDYQTYAELSVTNPYFTVDGVSLGGRLFYNDFQADDADLSDYTNKSYGTDVTLGFPINEYNSLRAGLGYVHNSLSNMQPQVAMWRYLYSMGEHPSTSDQDNSFKTDDFTFNYGWTYNKLDRGYFPTDGSRVNLTGKVTIPGSDNEYYKVTLDTATYVPIDDDHKWVVLGRTRWGYGDGLGGKEMPFYENFYAGGSSTVRGFQSNTIGPKAVYFPHQASNYDPDYDYECATQDGAKDLCKSDDAVGGNAMAVASLEFITPTPFISDKYANSVRTSFFWDMGTVWDTNWDSSQYSGYPDYSDPSNIRMSAGIALQWMSPLGPLVFSYAQPFKKYDGDKAEQFQFNIGKTW

SEQ ID NO: 32 - protein, 784 aa, Escherichia coli
MKKRIPTLLATMIATALYSQQGLAADLASQCMLGVPSYDRPLVQGDTNDLPVTINADHAKGDYPDDAVFTGSVDIMQGNSRLQADEVQLHQKEAPGQPEPVRTVDALGNVHYDDNQVILKGPKGWANLNTKDTNVWEGDYQMVGRQGRGKADLMKQRGENRYTILDNGSFTSCLPGSDTWSVVGSEIIHDREEQVAEIWNARFKVGPVPIFYSPYLQLPVGDKRRSGFLIPNAKYTTTNYFEFYLPYYWNIAPNMDATITPHYMHRRGNIMWENEFRYLSQAGAGLMELDYLPSDKVYEDEHPNDDSSRRWLFYWNHSGVMDQVWRFNVDYTKVSDPSYFNDFDNKYGSSTDGYATQKFSVGYAVQNFNATVSTKQFQVFSEQNTSSYSAEPQLDVNYYQNDVGPFDTRIYGQAVHFVNTRDDMPEATRVHLEPTINLPLSNNWGSINTEAKLLATHYQQTNLDWYNSRNTTKLDESVNRVMPQFKVDGKMVFERDMEMLAPGYTQTLEPRAQYLYVPYRDQSDIYNYDSSLLQSDYSGLFRDRTYGGLDRIASANQVTTGVTSRIYDDAAVERFNISVGQIYYFTESRTGDDNITWENDDKTGSLVWAGDTYWRISERWGLRGGIQYDTRLDNVATSNSSIEYRRDEDRLVQLNYRYASPEYIQATLPKYYSTAEQYKNGISQVGAVASWPIADRWSIVGAYYYDTNANKQADSMLGVQYSSCCYAIRVGYERKLNGWDNDKQHAVYDNAIGFNIELRGLSSNYGLGTQEMLRSNILPYQNTL

SEQ ID NO: 33 - protein, 172 aa, synthetic construct
  REGION 1..172: note = SlyB of SEQ ID NO:1 with a C-terminal TEV-His tag
MIKRVLVVSMVGLSLVGCVNNDTLSGDVYTASEAKQVQNVSYGTIVNVRPVQIQGGDDSNVIGAIGGAVLGGFLGNTVGGGTGRSLATAAGAVAGGVAGQGVQSAMNKTQGVELEIRKDDGNTIMVVQKQGNTRFSPGQRVVLASNGSQVTVSPRSGGGENLYFQGHHHHHH

SEQ ID NO: 34 - protein, 772 aa, synthetic construct
  REGION 1..772: note = Strep_His_TEV FhuA
MKKTAIAIAVALAGFATVAQAASWSHPQFEKSGGHHHHHHSGGGGGENLYFQGAVEPKEDTITVTAAPAPQESAWGPAATIAARQSATGTKTDTPIQKVPQSISVVTAEEMALHQPKSVKEALSYTPGVAVGTRGASNTYDYLIIRGFAADGQSQNNYLNGLKLQGNFYNDAVIDPYMLERAEIMRGPVSVLYGKSSPGGLLNMVSKRPTTEPLKEVQFKAGTDSLFQTGFDFSDALDDDGVYSYRLTGLARSANAQQKGSEEQRYAIAPAFTWRPDDKTNFTFLSYFQNEPETGYYGWLPKEGTVEPLPNGKRLPTDFNEGAKNNTYSRNEKMIGYSFDHEFNDTFTVRQNLRFAQNKVSQKSVYGYGMCSDPLYTKDQEALKASPCLSIPQSQWGHTLTRQYVIDNEKLENFSVDTQLQSKFATGSVDHTLLTGVDFMRMRNDIDSWFGYAGSVAPSDIYNLDRSDFDFGAHPNPSGPYRVLLKQKQTGLYVQDQAQWDKVLVTLGGRYDWADQSSFNRDYGNKSERDDKEFTWRGGVNYLFDNGVTPYFSYSESFEPASQTDANGDLFAPSKGKQYEVGVKYVPEDRPIVVTGALYQLTKTNNLMADPNGSLFSVEGGEIRARGVELEAKAALSASVNVVGSYTYTDAEYTTDTTYKGNTPAQVPKHMASLWADYTFFDGPLSGLTLGTGGRYTGSSYGDPANSFKVGSYTVVDALVRYDLARVGMAGSNVALHVNNLFDREYVASCFNTYGCFWGAERQVVATATFRF

SEQ ID NO: 35 - protein, 647 aa, synthetic construct
  REGION 1..647: note = Strep_His_TEVBtuB
MKKTAIAIAVALAGFATVAQAASWSHPQFEKSGGHHHHHHSGGGGGENLYFQGQDTSPDTLVVTANRFEQPRSTVLAPTTVVTRQDIDRWQSTSVNDVLRRLPGVDITQNGGSGQLSSIFIRGTNASHVLVLIDGVRLNLAGVSGSADLSQFPIALVQRVEYIRGPRSAVYGSDAIGGVVNIITTRDHPGTEISAGWGSNSYQNYDVSTQQQLGDKTRVTLLGDYAHTHGYDVVAYGNTGTQAQPDNDGFLSKTLYGALEHNFTDAWSGFVRGYGYDNRTNYDAYYSPGLPLVDTRKLYSQSWDAGLRYNGELIKSQLITSYSHSKDYNYDPHYGRYDSSATLDEMKQYTVQWANNIIIGHGNIGAGVDWQKQSTAPGTAYVEDGYDQRNTGIYLTGLQQVGDFTFEGAGRSDDNSQFGRHGTWQTSAGWEFIEGYRFIASYGTSYKAPNLGQLYGSYGNPNLNPEKSKQWEGAFEGLTAGVNWRISGYRNDVSDLIDYDDHTLKYYNEGKARIKGVEATANFDTGPLTHTVSYDYVDARNAITDTPLLRRAKQQVKYQLDWQLYDFDWGITYQYLGTRYDKDYSSYPYQTVKMGGVSLWDLAVAYPVTSHLTVRGKIANLFDKDYETVYGYQTAGREYTLSGSYTF

SEQ ID NO: 36 - protein, 843 aa, synthetic construct
  REGION 1..843: note = Strep_His_TEVBamA
MKKTAIAIAVALAGFATVAQAASWSHPQFEKSGGHHHHHHSGGGGGENLYFQGAEGFVVKDIHFEGLQRVAVGAALLSMPVRTGDTVNDEDISNTIRALFATGNFEDVRVLRDGDTLLVQVKERPTIASITFSGNKSVKDDMLKQNLEASGVRVGESLDRTTIADIEKGLEDFYYSVGKYSASVKAVVTPLPRNRVDLKLVFQEGVSAEIQQINIVGNHAFTTDELISHFQLRDEVPWWNVVGDRKYQKQKLAGDLETLRSYYLDRGYARFNIDSTQVSLTPDKKGIYVTVNITEGDQYKLSGVEVSGNLAGHSAEIEQLTKIEPGELYNGTKVTKMEDDIKKLLGRYGYAYPRVQSMPEINDADKTVKLRVNVDAGNRFYVRKIRFEGNDTSKDAVLRREMRQMEGAWLGSDLVDQGKERLNRLGFFETVDTDTQRVPGSPDQVDVVYKVKERNTGSFNFGIGYGTESGVSFQAGVQQDNWLGTGYAVGINGTKNDYQTYAELSVTNPYFTVDGVSLGGRLFYNDFQADDADLSDYTNKSYGTDVTLGFPINEYNSLRAGLGYVHNSLSNMQPQVAMWRYLYSMGEHPSTSDQDNSFKTDDFTFNYGWTYNKLDRGYFPTDGSRVNLTGKVTIPGSDNEYYKVTLDTATYVPIDDDHKWVVLGRTRWGYGDGLGGKEMPFYENFYAGGSSTVRGFQSNTIGPKAVYFPHQASNYDPDYDYECATQDGAKDLCKSDDAVGGNAMAVASLEFITPTPFISDKYANSVRTSFFWDMGTVWDTNWDSSQYSGYPDYSDPSNIRMSAGIALQWMSPLGPLVFSYAQPFKKYDGDKAEQFQFNIGKTW

SEQ ID NO: 37 - protein, 407 aa, synthetic construct
  REGION 1..407: note = OmpC2xStrep
MMKVKVLSLLVPALLVAGAANAAEVYNKDGNKLDLYGKVDGLHYFSDNKDVDGDQTYMRLGFKGETQVTDQLTGYGQWEYQIQGNSAENENNSWTRVAFAGLKFQDVGSFDYGRNYGVVYDVTSWTDVLPEFGGDTYGSDNFMQQRGNGFATYRNTDFFGLVDGLNFAVQYQGKNGNPSGEGFTSGVTNNGRDALRQNGDGVGGSITYDYEGFGIGGAISSSKRTDAQNTAAYIGNGDRAETYTGGLKYDANNIYLAAQYTQTYNATRVGSLGWANKAQNFEAVAQYQFDFGLRPSLAYLQSKGKNLGRGYDDEDILKYVDVGATYYFNKNMSTYVDYKINLLDDNQFTRDAGINTDNIVALGLVYQFGGGSGGGGSGWSHPQFEKSGGSGGSGGGSSAWSHPQFEK

SEQ ID NO: 38 - protein, 653 aa, synthetic construct
  REGION 1..653: note = BtuB2xStrep
MIKKASLLTACSVTAFSAWAQDTSPDTLVVTANRFEQPRSTVLAPTTVVTRQDIDRWQSTSVNDVLRRLPGVDITQNGGSGQLSSIFIRGTNASHVLVLIDGVRLNLAGVSGSADLSQFPIALVQRVEYIRGPRSAVYGSDAIGGVVNIITTRDEPGTEISAGWGSNSYQNYDVSTQQQLGDKTRVTLLGDYAHTHGYDVVAYGNTGTQAQTDNDGFLSKTLYGALEHNFTDAWSGFVRGYGYDNRTNYDAYYSPGSPLLDTRKLYSQSWDAGLRYNGELIKSQLITSYSHSKDYNYDPHYGRYDSSATLDEMKQYTVQWANNVIVGHGSIGAGVDWQKQTTTPGTGYVEDGYDQRNTGIYLTGLQQVGDFTFEGAARSDDNSQFGRHGTWQTSAGWEFIEGYRFIASYGTSYKAPNLGQLYGFYGNPNLDPEKSKQWEGAFEGLTAGVNWRISGYRNDVSDLIDYDDHTLKYYNEGKARIKGVEATANFDTGPLTHTVSYDYVDARNAITDTPLLRRAKQQVKYQLDWQLYDFDWGITYQYLGTRYDKDYSSYPYQTVKMGGVSLWDLAVAYPVTSHLTVRGKIANLFDKDYETVYGYQTAGREYTLSGSYTFGGGSGGGGSGWSHPQFEKSGGSGGSGGGSSAWSHPQFEK

SEQ ID NO: 39 - protein, 333 aa, synthetic construct
  REGION 1..333: note = Tsx2xStrep
MKKTLLAAGAVLALSSSFTVNAAENDKPQYLSDWWHQSVNVVGSYHTRFGPQIRNDTYLEYEAFAKKDWFDFYGYADAPVFFGGNSDAKGIWNHGSPLFMEIEPRFSIDKLTNTDLSFGPFKEWYFANNYIYDMGRNKDGRQSTWYMGLGTDIDTGLPMSLSMNVYAKYQWQNYGAANENEWDGYRFKIKYFVPITDLWGGQLSYIGFTNFDWGSDLGDDSGNAINGIKTRTNNSIASSHILALNYDHWHYSVVARYWHDGGQWNDDAELNFGNGNFNVRSTGWGGYLVVGYNFGGGSGGGGSGWSHPQFEKSGGSGGSGGGSSAWSHPQFEK

SEQ ID NO: 40 - DNA, 70 bp, synthetic construct
  misc_feature 1..70: note = Primer
  source 1..70: mol_type = other DNA
atgagggatgcttgcgtggcgtttcatggtgaacaggagatttttcaatggtgtaggctggagctgcttc

SEQ ID NO: 41 - DNA, 70 bp, synthetic construct
  misc_feature 1..70: note = Primer
  source 1..70: mol_type = other DNA
gtcagtaacctgaccacacgcaaacttatttagcgcggagaaacggtcaccatatgaatatcctccttag

SEQ ID NO: 42 - DNA, 70 bp, synthetic construct
  misc_feature 1..70: note = Primer
  source 1..70: mol_type = other DNA
tatagggatgctgaaatagcgtttcgtgaagaacaggagatgggtaaatggtgtaggctggagctgcttc

SEQ ID NO: 43 - DNA, 70 bp, synthetic construct
  misc_feature 1..70: note = Primer
  source 1..70: mol_type = other DNA
tggccatgtgaccacacgtaaagcctggtttagcgtggagagacggtaaccatatgaatatcctccttag

SEQ ID NO: 44 - DNA, 70 bp, synthetic construct
  misc_feature 1..70: note = Primer
  source 1..70: mol_type = other DNA
cgggaaaaaaagacgcagtaattttttcatcagcgcaattcgaacagatagtgtaggctggagctgcttc

SEQ ID NO: 45 - DNA, 70 bp, synthetic construct
  misc_feature 1..70: note = Primer
  source 1..70: mol_type = other DNA
ataatcgcgttacactattttaataattaagacagggagaaataaaaatgcatatgaatatcctccttag

SEQ ID NO: 46 - DNA, 27 bp, synthetic construct
  misc_feature 1..27: note = Primer
  source 1..27: mol_type = other DNA
accgtgggtggcttccagtccaatacc

SEQ ID NO: 47 - DNA, 27 bp, synthetic construct
  misc_feature 1..27: note = Primer
  source 1..27: mol_type = other DNA
gaagccacccacggtgctggaaccacc

SEQ ID NO: 48 - DNA, 33 bp, synthetic construct
  misc_feature 1..33: note = Primer
  source 1..33: mol_type = other DNA
ttctggggtatgggtaccgtttgggatacaaac

SEQ ID NO: 49 - DNA, 31 bp, synthetic construct
  misc_feature 1..31: note = Primer
  source 1..31: mol_type = other DNA
acccataccccagaagaaggaagtacgaacc

SEQ ID NO: 50 - skipped

SEQ ID NO: 51 - protein, 49 aa, Escherichia coli
GDDSNVIGAIGGAVLGGFLGNTVGGGTGRSLATAAGAVAGGVAGQGVQS

SEQ ID NO: 52 - protein, 53 aa, Escherichia coli
EAGKSAIGAGLGSLVGAGIGALSSSKKDRGKGALIGAAAGAALGGGVGYYMDV

SEQ ID NO: 53 - protein, 49 aa, Escherichia coli
QDENRITGSVLGAVAGGVIGHQFGGGRGKDVATVVGALGGGYAGNQIQG

SEQ ID NO: 54 - protein, 42 aa, Escherichia coli
DMNRTTKGALLGAGVGLLTGNGVNGVLKGAAVGAGVGAVTEK

SEQ ID NO: 55 - protein, 43 aa, Escherichia coli
RDRNTAIGAGAGALGGAVLTDGSTLGTLGGAAVGGVIGHQVGK

SEQ ID NO: 56 - protein, 51 aa, Escherichia coli
KRNAQAFGALIGAVAGGVIGHNVGSGSNSGTTAGAVGGGAVGAAAGSMVND

SEQ ID NO: 57 - protein, 138 aa, Escherichia coli
CVNNDTLSGDVYTASEAKQVQNVSYGTIVNVRPVQIQGGDDSNVIGAIGGAVLGGFLGNTVGGGTGRSLATAAGAVAGGVAGQGVQSAMNKTQGVELEIRKDDGNTIMVVQKQGNTRFSPGQRVVLASNGSQVTVSPR

SEQ ID NO: 58 - protein, 138 aa, Serratia sp.
CANTSTLSGDVYSASDAKQVQTVTYGTIISTRPVQIQAGEENNIIGALGGAVLGGFLGNTVGGGTGRSLATAAGAVAGGVAGQSVEGAINRVQGVQLEIRKDDGNTIMVVQKQGTTKFYAGQRIAMVSDGRSVTVSPR

SEQ ID NO: 59 - protein, 138 aa, Erwinia sp.
CVNDSSLSGDVYSASEAKQIQNVTYGTLVSVRPVQIQGDDNTNVIGAIGGAVLGGFLGNTVGGGTGRSLATAAGAVAGGVAGQGVQSSMNKSQGVELEIRRDDGSTIAVVQKQAASRYSVGQRVAMTSNGREVTVSPR

SEQ ID NO: 60 - protein, 138 aa, Edwardsiella tarda
CANTSTLSGDVYSANQAKQVQQVSYGTIVSIEPVRIQAGNEANMIGTIGGAVIGGLLGNTIGGGTGRSLATAAGAVAGGVAGNSIEGAVNRTDGVQLVIKMDSGKTIAVVQKADKQAFRVNQRVMLLSNGSSVNVSPM

SEQ ID NO: 61 - protein, 138 aa, Serratia odorifera
CANDSMSGDVYSASQAKQVQQASYGTLVSVRPVKISGEGGSNMLGTIGGAVIGGFLGNTIGGGTGRSLATAAGAVAGGVAGDQIGEAAGRTSGVELEIRPDTKGDNIIVVQKAGTTKFSPGQRVRMARSGSTITVSPL

SEQ ID NO: 62 - protein, 138 aa, Proteus penneri
CVNTSSLSGDTYTASQAKQAQNITYGTVVSVRSVNIQAGSDENILGAIGGAVLGGMLGNTVGGGTGRNLATAAGAIAGGMAGQQAQGALNTTKGVQIEVRLDSGRTVAIVQKADNTVYSKGQRVAVIGNGNNLTVSPR

SEQ ID NO: 63 - protein, 140 aa, Xenorhabdus nematophilus
CADMGALSSDTYSMDQAKQAQTVTYGTILSVRPVTIKGNQAGDPNVLGLIGGAVLGGLLGNTVGGGSGQRLATAAGAIAGGMAGQRIEGAIDQTKGVELEIRTDSGKNIIVVQKLDQVVFNRGQRVRIANSGNSLTVSPR

SEQ ID NO: 64 - protein, 149 aa, Gilliamella bombicola
CSNSDLYSGDVYTADQAKQVQQVSYGTVVSVRPVKIQANASSDGKSENVAGNLGGAVLGGVLGNTIGGGTGRVLAVATGAIGGAILGDTIQDKTSQASAVELEIKQENGSNIVIVQKGSAKDFFAGQAVRLVTNGKKISASPRYNNQSK

SEQ ID NO: 65 - protein, 138 aa, Halomonas anticariensis
CANTAPYSGDVYRGSQAQTGQTVSYGTIVALRQVQIQADNPQGNVVGGLGGAVIGGLLGNQIGGGSGRTLATAAGAIGGSVAGSKIEDRANRTTAWEMEIRRDRGDNVIVVQKADRAFQVGQRVRLIGSGANLSVAPY

SEQ ID NO: 66 - protein, 138 aa, Oceanisphaera profunda
CANPDVYSGDVYGSRAAKQAQTVTYATVTSVRPVLIQAGDENPNVIGTLGGGAIGGILGHQVGGGTGQVLATAVGALAGTMVGSRAEDKLSQVKAVELEVRTDSGENLIVVQKADRTWQPGQRVRLVGSGSNLSVAPN

SEQ ID NO: 67 - protein, 137 aa, Aeromonas molluscorum
CANSDVYSGDVYTKDRAKQVQTVSYGTIVSTRPVKIQAEENSILGTIGGGVVGGLLGSTIGGGTGRSLAAAGGAIAGAAAGKAAGDKLNQVDGVELEIKQDNGSSIVVVQKADPSFQPGAKVRLTQGNGSVNAAVVP

SEQ ID NO: 68 - protein, 135 aa, Haemophilus pittmaniae
CANTDVFSGDVYTADQAKEARSISYGTILSVRPVKIQADNPGVIGTIGGGALGGIAGSTVGGGTGQALATAVGAIAGAVVGSKVEEKASQVNAAELVIRKDNGKQIVVVQKADPNFVAGRRVRIVGGSSLNVSVL

SEQ ID NO: 69 - protein, 136 aa, Simonsiella muelleri
CANKSIYSGNVYTSSQAKETRAISYGTIVSVREVQIQAPSSGVIGTTAGGALGGIAGSSMGGGRGSDLLGVLVGTVGAIVGNQIEQGMSKVASLEMVIRKDDGAEIVVVQKKEAGLTVGKRVRLVGSASDVNVSPL

SEQ ID NO: 70 - protein, 138 aa, Shewanella halifaxensis
CTTPTPYGDAYAASETRTIQQVYYGTIVKAEPVTIAASDKTNIIAMIAGAAIGGILGSEVGGGAGSDIAAIGGSVAGGYAGSEIADAAGERNGVNLTIRLEDGRIISIVQEVNPSMIFQQGQAVQVNVDGNTARVVPR

SEQ ID NO: 71 - protein, 134 aa, Pseudogulbenkiania sp.
CATSDSAAVYSKGQMRQAQNVEFGTVVSVQNVLMEGNNNELLTLGGAALGGIAGSTIGQGRGAAAGTIVGALAGGFGTQAAQRNLGTKNALEITVKLDRSGRMLSIVQEADIPFAPGQRVKVLTGGGNDRVAPL

SEQ ID NO: 72 - protein, 138 aa, Klebsiella variicola
CTNTSHLSGDVHSSATAGRVQQVFYGQLVSVRAVQIQAGDNSNALGAVAGGVIGGVAGSAVGGGSGQRLATATGAVAGGLAGQAVQARMNLVQGVELEIRLDDGRTFMVVQRQGPTRFSVGQRVAISSVGGQTTVSPR

SEQ ID NO: 73 - protein, 139 aa, Klebsiella pneumoniae
CTEKPVGSAHHASAALSGQVMALSHGTITGVRYITIQNDTGSSAAGTVGGALIGGAAGNAIGGGSGRLLATAGGTVAGAMAGQSIQRGMARHNAAELFIRPDNGQEFVTVQPAREGEFVVGQRVNITSHRGQVTISPAQ

SEQ ID NO: 74 - protein, 135 aa, Bordetella avium
CANHSASSSVYTYGQAQREQIVRTGTVTGVRPITIQTDKSSGVGALAGGALGGVAGNAIGGGTGRTIATVGGAILGALAGNVAENQVGKTSGYEITVRLDNGETRVVAQEADNPVSVGQRVQVISGAGPTRVVPY

SEQ ID NO: 75 - protein, 135 aa, Pusillimonas sp.
CANDNASSSVYSYGQAQREQIVRTGTVEAVRNVTIQKDSTSGAGMIAGGALGGVAASTIGGGTGQVLATIGGGILGAMAGNAVENQVGKTKGLEITVRLDNGETRVIAQAADVPVGVGQRVRLISGNGPTRVVPM

SEQ ID NO: 76 - protein, 135 aa, Achromobacter arsenitoxydans
CANPSASSRVYTFDQAQRAQTVTLGYVVGVRPVTIQASQTSGLGVMAGGALGAVTGSAIGGGSGRRITTVGGAVGGAMAGNAIENASARRAGLELTVRLDSGDLRVITQEADVPLSTGQRVQIVTQGGVSRVVGI

SEQ ID NO: 77 - protein, 137 aa, Paraburkholderia phytofirmans
CAYNSSSADVYTASQAQREETVRMGTVDSVRAVKISSNNGQPSGLGAIGGGALGAVAGSAIGGGRGSIVTGIIGGLAGAVAGNAVENGAAVHDGLEITVRLDNGDMRAITQSATGEIFRAGERVRLLSSGGVTRVTH

SEQ ID NO: 78 - protein, 136 aa, Burkholderia cepacia
CITPPGSADVYSSGQAQREQTVRMGTVESVRATRIQGSDGGSVLGSLGGGALGAVAGSAIGGGRGSVLTTIAGGIAGAVAGNAVGQGLSSANGVEITVRLDNGDLRSIAQAATPETFRAGDRVRLLSSAGVTRVTH

SEQ ID NO: 79 - protein, 133 aa, Janthinobacterium agaricidamnosum
CAVAPNSNTVYTTRQAQSEQSVRLATVESVRQVTIDKGSSGTGVLAGAALGGLAGSTVGGGKGSIATSIIGAIAGGAVGQNVEANVNNKPGFEITVRLDNGELRSIVQDADEAFRPGERVRLLSDGRKTRVTH

SEQ ID NO: 80 - protein, 137 aa, Sedimenticola thiotaurini
CTTSGLSGTTYSRTDARKVQTVKYGTIESVTPVVIEGRTDGIVGTGSGAVVGGIAGSTVGGGKGRTIAAVVGAVAGGLAGQAAEKKLTTKQGQELTIRLEATGDIISVVQEVENEQFFRPGDRIRLLQYGGNTRVAY

SEQ ID NO: 81 - protein, 134 aa, Pseudomonas fulva
CASSLTGDSYSRDEARAVQTVRMGTIESLRPVKIEGTKTPIGGGAGAVIGGVAGSGVGGGRGSAVAAVIGAVAGGLLGAAAEEGITRTQGVEITVREDDGSMRAYVQAVEENQIFRVGDRVRIMTVNGTSRVTH

SEQ ID NO: 82 - protein, 134 aa, Pseudomonas alcaligenes
CASNLNGSTYSRADARAVQTVRLGTIESLRPVQIEGTRTPIGAIAGAAVGGIAGSGVGGGRGSSIATVLGGVAGGMAGAAAEEGLTRSQGVEITVREDDGNLRAYVQEVDPAQQFRVGDRVRIASVNGASRVTL

SEQ ID NO: 83 - protein, 132 aa, Methylovorus glucosetrophus
CASSNSGSVYSRDEARKVQTVKMGVVESVRTVKLEGTKSPVGTAGGAVVGGVAGSTMGGGKGQAIATVLGAIVGGLAGSAAEEGLTRKDGLEITVKLDSGTMIAVVQEADDQFQPGERVRILESGGTTRVSH

SEQ ID NO: 84 - protein, 133 aa, Thauera sp.
CAQGLGGGTYSRTEARRAMTVQFGTVESVRGVQLEGTKSPVGTVAGAAVGGIAGSTIGGGRGQAVATVIGAVAGGVAGSALEEGMTRTPGVEVTVRLDNGQFLAVVQADEGERLRPGERVRVLRDGGTTRVTR

SEQ ID NO: 85 - DNA, 25 bp, synthetic construct
  source 1..25: mol_type = other DNA
caccgctagcgatacgctgaccgac

SEQ ID NO: 86 - DNA, 28 bp, synthetic construct
  source 1..28: mol_type = other DNA
gtatcgctagcggtgaggatcagtaccg

SEQ ID NO: 87 - DNA, 70 bp, synthetic construct
  source 1..70: mol_type = other DNA
cataatcaacgctagactgttcttattgttaacacaagggagaagagatggtgtaggctggagctgcttc

SEQ ID NO: 88 - DNA, 70 bp, synthetic construct
  source 1..70: mol_type = other DNA
cggcagaaaatggcgagcaaatttattcattagcgcaattcaaaaagatacatatgaatatcctccttag

SEQ ID NO: 89 - protein, 15 aa, synthetic construct
SGGHHHHHHSGGGGG

(19) United States
(12) Patent Application Publication
(10) Pub. No.: US 2026/0115279 A1
(43) Pub. Date: Apr. 30, 2026

(54) Methods for Isolation of Lipid-Disc Compositions and Uses Thereof

(71) Applicants: VIB VZW, Gent (BE); Vrije Universiteit Brussel, Brussel (BE)

(72) Inventors: Han Remaut, Roosbeek (BE); Van-Son Nguyen, Brussel (BE); Arne Janssens, Huldenberg (BE)

(21) Appl. No.: 18/688,085

(22) PCT Filed: Aug. 30, 2022
(86) PCT No.: PCT/EP2022/074095

§ 371 Date: Feb. 29, 2024

(30) Foreign Application Priority Data
Aug. 30, 2021 (EP) 21193811.3
Apr. 20, 2022 (EP) 22169108.2

(51) Int. Cl. A61K 39/385; A61K 9/1272; C12N 15/70; C12P 21/02

(52) U.S. Cl. CPC A61K 39/385; A61K 9/1272; C12N 15/70; C12P 21/02; C12N 2800/101

(57) ABSTRACT

The invention relates to the field of bacterial membrane protein structures. More specifically, the invention relates to lipid nanodiscs compartmentalized by SlyB protein oligomers isolated from the outer membrane of Gram-negative bacteria. More specifically, the invention provides for a SlyB nanodisc structure wherein the SlyB-oligomer forms the membrane scaffold protein belt, which is surrounded by outer saccharolipid moieties anchored to the SlyB proteins, and which encloses a lipid bilayer nanodomain containing one or more phospholipid layers, wherein macromolecules such as (outer) membrane protein molecules may be captured and stabilized. More specifically, methods to produce and isolate chemically defined stable SlyB nanodisc particles are disclosed herein. Finally, the invention relates to the use of said SlyB nanodiscs as a self-adjuvanting vehicle, as part of an immunogenic composition, and provides for novel means for use in eliciting an immune response against macromolecules enclosed in said SlyB nanodiscs, or for use in a vaccine composition.

### CROSS REFERENCE TO RELATED APPLICATIONS

This application is a national phase entry under 35 U.S.C. § 371 of International Patent Application PCT/EP2022/074095, filed Aug. 30, 2022, designating the United States of America and published in English as International Patent Publication WO2023/031205 on Mar. 9, 2023, which claims the benefit under Article 8 of the Patent Cooperation Treaty to European Patent Application Serial No. 21193811.3, filed Aug. 30, 2021, and European Patent Application Serial No. 22169108.2, filed Apr. 20, 2022, the entireties of which are hereby incorporated by reference.

### INCORPORATION BY REFERENCE

The ST.26 XML Sequence listing named “10371 US 2025-03-07—Substitute Sequence Listing ST26.xml”, created on Mar. 7, 2025, and having a size of 98,267 bytes, is hereby incorporated herein by this reference in its entirety.

### FIELD OF THE INVENTION

The invention relates to the field of bacterial membrane protein structures. More specifically, the invention relates to lipid nanodiscs compartmentalized by SlyB protein oligomers isolated from the outer membrane of Gram-negative bacteria. More specifically, the invention provides for a SlyB nanodisc structure wherein the SlyB-oligomer forms the membrane scaffold protein belt, which is surrounded by outer saccharolipid moieties anchored to the SlyB proteins, and which encloses a lipid bilayer nanodomain containing one or more phospholipid layers, wherein macromolecules such as (outer) membrane protein molecules may be captured and stabilized. More specifically, methods to produce and isolate chemically defined stable SlyB nanodisc particles are disclosed herein. Finally, the invention relates to the use of said SlyB nanodiscs as a self-adjuvanting vehicle, as part of an immunogenic composition, and provides for novel means for use in eliciting an immune response against macromolecules enclosed in said SlyB nanodiscs, or for use in a vaccine composition.

### INTRODUCTION

Gram-negative bacteria are surrounded by a cytoplasmic or ‘inner’ membrane and the protective outer membrane (OM) layer, the latter being highly asymmetric and composed from integral outer membrane β-barrel proteins, lipoproteins, lipopolysaccharides, and phospholipids. While these components are synthesized in the cytoplasm of the bacterial cell, translocation over the inner membrane and passage through the periplasm is required to result in an outer membrane localization, which occurs by specific translocator and assembly systems. The established outer membrane environment containing a dynamic mixture of components including outer membrane proteins has been considered as a source of bacterially derived membrane protein structures which can be isolated from those bacterial cells and having physical properties making them applicable and suitable for a number of applications, such as for instance as a structural chaperone tool, to provide a hydrophobic environment, or to stabilize certain components. Several bacterially derived membrane structures are known in the art, such as ‘nanodiscs’ providing for a synthetic non-covalent structure of phospholipid bilayer and membrane scaffold proteins, which are typically represented by genetically engineered protein mimics of Apolipoprotein A-1; or ‘liposome’ particles or vesicles known as vehicle or carrier for cargo delivery systems; or ‘Bacterial Outer Membrane Vesicles’ (OMV), which are spherical vesicles with a bilayered proteolipid structure that are naturally secreted from the Gram-negative bacteria by blebbing of the outer cell membrane. OMVs are associated with various biological functions, such as roles in pathogenesis, cell-to-cell communication, increased tolerance to stress and antibiotics, as reviewed in detail in ref [51]. OMVs present numerous proteins on their surface, resembling their parent bacterial membrane, which makes them promising vaccine alternatives, for instance in prevention of bacterial infections, and beyond, as platform to develop vaccines against different types of pathogens. The first and most representative OMV-based vaccines target Neisseria meningitidis serogroup B (MenB), through OMVs containing several variants of PorA, an outer membrane protein (OMP) of said pathogen. MenB vaccines (e.g., VA-MENGOC-BC, MenBvac, and MeNZB) successfully combated several outbreaks of MenB-caused meningitis in Cuba, Norway, and New Zealand [52]. Despite the promises OMV based vaccines hold, they also have several significant disadvantages, such as safety and low production rate that restricts their wide usage as vaccines. Indeed, the endotoxic component of LPS, Lipid A, as present in Gram-negative outer membranes, and thus in OMVs can cause a severe, even lethal inflammatory response when administered to a subject. By applying genetically detoxified strains however, modified in their lipid A synthesis [53, 51], detoxified OMVs provided already for a solution to the safety issue. In addition, the release of outer membrane vesicles is difficult to control, with a manufacturing yield often too low and/or unpredictable for manufacturing purposes. Detergent-based isolation of OMVs and/or the use of genetically modified bacteria targeting genes, for example, involved in crosslinking the outer membrane and peptidoglycan layer in the periplasm (e.g., Tol-Par system), have been proposed for increasing the blebbing and final yield. However, such solutions also bring their disadvantages. Detergent-induced OMVs often lose the lipoprotein antigens, the vesicle integrity may be compromised, and cytoplasmic protein contamination occurs. Disturbing the Tol-Pal system, on the other hand, can lead to a defect in the polymerization of LPS O-antigen, a key target of protective immunity [54,55]. Furthermore, OMV cargo varies between strains, which may, in some cases, limit their applicability to a specific subset of strains. One solution is to bioengineering recombinant OMVs with exogenic antigens, recombinant strains carrying antigens fused to a native OMV proteins such as ClyA to pull them to the cell surface [56]. Bioengineered OMVs significantly broadens the utilization of OMVs vaccines, from against limited pathogens to bacterial, viral, and tumor vaccines. The number of exogenous antigens presented at the OMV surface and, consequently, the efficiency of the OMV vaccine will however still be considerably limited by the native OMV proteins (e.g., ClyA) anchoring to the outer membrane, as well as the number of OMVs secreted by the host cell. Current unmet needs have been tackled by, for instance, the use of high-pressure homogenization technology in an attempt to produce ring-like vesicles from antigen-ClyA presenting bacterial membrane [57].

The application of lipid nanoparticles, such as nanodiscs, in delivery of hydrophobic chemotherapy agents was shown to be of benefit in view of bioavailability and stabilization, or delivery of hydrophilic molecules, as well as the use of adjuvant-loaded nanodiscs in cancer vaccination and immunotherapy. These findings position nanodiscs as a powerful vehicle in pharmaceutical applications (e.g. Kuai et al., 2018; J. Controlled Release 282, 131-139; Bariwal et al., 2022; Chem. Soc. Rev., 2022, 51, 1702). Though, the ApoI-based MSPs of currently used nanodiscs are disadvantageous in their production cost, long-term stability and clinical safety, requiring further engineering or improved alternatives.

Overall, despite many advancements in the application of bacterial membrane structures as chaperone tool, or as immunogen, adjuvant, delivery or vaccine vehicle in pharmaceutical applications, there is still no optimal and generic approach to efficiently produce the desired structures and overcome the disadvantages discussed herein. Recent improvements on using OMV-based and nanoparticle-based bacterial outer membrane structures for cancer vaccine development as for instance demonstrated by Zhao et al. (Not Protoc (2022). https://doi.org/10.1038/s41596-022-00713-7) are intriguing for further innovation into designing the most optimal bacterial outer membrane modalities for applications in vaccine and therapeutic development within optimal spectra of the desired and required properties.

By finding novel ways to produce and isolate improved bacterially-originating membrane protein structures, and further applications in uses thereof, such as novel stabilizing cargo-chaperones, which can be chemically defined, and hence safely act as vaccine vehicles, adjuvants, or immunogens, would further advance this technological field.

### SUMMARY OF THE INVENTION

The present invention is based on the finding that isolation of outer membrane lipoproteins and β-barrel proteins from Gram-negative bacteria, when performed under certain stress conditions, results in encapsulation of said outer membrane proteins within a nanoscale lipid domain that is enclosed by an oligomeric ring of SlyB lipoproteins, which basically act as membrane scaffolding proteins forming a discoidal structure around and protecting those outer membrane proteins (OMPs). The present invention further reveals generic methods to extract those SlyB:OMP nanodomain complexes from the bacterial outer membrane using detergents, as to obtain soluble nanodiscs, which provide for a novel nanodisc structure, derived from the bacterial outer membrane, those resulting particles hereafter referred to as “SlyB nanodiscs”.

SlyB is a component of the PhoPQ stress regulon, and is constituted of a 10 kDa periplasmic 3-sandwich domain and a glycine zipper domain that forms a transmembrane α-helical hairpin with a discrete LPS and phospholipid binding site. The recombinant production of BamA, an E. coli membrane β-barrel protein, revealed stable circular entities formed by SlyB monomers oligomerizing into ring-shaped transmembrane complexes that encapsulate β-barrel proteins such as BamA into lipid nanodomains of variable size. SlyB nanodomains were found to be formed as stress-induced complexes with the outer membrane proteome, thereby stabilizing enclosed outer membrane proteins in vitro and in vivo. When extracted from the outer membrane, said nanodomains form detergent soluble nanoscale particles called “SlyB outer membrane discs (SlyB OMDs)”, “SlyB lipid discs”, SlyB lipid nanodiscs”, or “SlyB nanodiscs”, as used interchangeably herein, which comprise a circular SlyB oligomer that encloses a lipid bilayer and its embedded membrane proteins. Lipopolysaccharide destabilization by antimicrobial peptides or cation shortage, conditions that induce the PhoP-PhoQ stress regulon in Gram-negative bacterial cells, but also disruptions in LPS biosynthesis, resulted in an increased expression of SlyB and unexpectedly in the formation of SlyB nanodomains as described herein. So, SlyB nanodomain formation is essential during lipopolysaccharide destabilization by antimicrobial peptides or cation shortage, conditions that result in a loss of OMPs and localized OM breaches in absence of a functional SlyB. SlyB proteins represent a larger family of broadly conserved (lipo)proteins with 2™ glycine zipper domains capable of forming lipid nanodomains. So, SlyB oligomer compositions are provided herein as compartmentalizing transmembrane chaperone or membrane scaffolding compositions. The production of soluble SlyB nanodiscs, in contrast to the costly and synthetic production of other nanodiscs, is simply obtained by applying specific SlyB nanodomain-inducing culturing conditions of Gram-negative bacterial cultures containing a SlyB-encoding gene, recombinantly introduced, or endogenously in their genome, and applying detergent solubilization methods to extract SlyB nanodomains form the bacterial membrane. Interestingly, this induction mechanism even further enabled to develop a generic production method of recombinant SlyB expression for formation of a nanodisc composition carrying encapsulated targets of interest, thereby providing a stable environment for the protein of interest within said SlyB nanodisc. Said recombinantly produced nanodisc particles are thus produced in a controlled manner, and consequently purified as isolated SlyB nanodisc particles using detergent-mediated extraction and preferably size-separation techniques, resulting in novel stabilizing nanodiscs for membrane proteins or other enclosed macromolecules, and thereby acting as vehicle or carrier to deliver ‘cargo’ molecule(s). These findings were strengthened by the observation that the isolated SlyB nanodiscs from E. coli recombinant cultures were capable to act as self-adjuvanting vaccine vehicle, eliciting an immune response in mice against nanodisc-embedded outer membrane proteins of interest, demonstrating their potential to utilize the SlyB-based nanodisc lipid particles in the biomedical field as vehicle carrier, as adjuvant, or even as a vaccine composition, besides its application as a tool in structural and biophysical analysis of membrane protein targets, or as an aid in purification.

The first aspect of the present invention relates to a SlyB lipid nanodisc composition, isolated from a host cell, preferably from a bacterial host cell, which comprises at least the following elements:
one or more oligomers formed by SlyB protomers, wherein each oligomer forms a circular or discoidal structure contains at least two or more SlyB protein subunits, preferably between 2 and 25 monomers, and preferably wherein the SlyB amino acid sequence is presented by the mature protein form of any one of the SlyB proteins of SEQ ID NO:1-28 (such as the mature proteins depicted in SEQ ID NOs: 57-84, resp.) or a bacterial homologue with a mature domain of at least 80% identity of any one thereof, wherein the % identity is determined over the full length of the mature domain of said SlyB sequence of SEQ ID NO: 1-28 (i.e. after removal of the N-terminal signal sequence, as shown in SEQ ID NOs: 57-84), or wherein at least one or more SlyB subunits of said oligomer may be a SlyB mutant, or a functional SlyB variant;
said SlyB oligomer enclosing a lipid nanodomain on its luminal side, which contains at least one or more phospholipid layers, ultimately lining the oligomer, and anchored to the SlyB oligomer, said inner lipid nanodomain possibly containing one or more proteins captured within said phospholipid layer(s); and
the external lining of said SlyB oligomer binding at least one molecule forming a saccharolipid moiety surrounding the SlyB oligomeric disc or belt, preferably one saccharolipid moiety for each SlyB protomer of said oligomer.

The isolated SlyB nanodisc composition described herein may thus contain one SlyB nanodisc, or preferably, a multiplicity of SlyB nanodiscs. Said multiplicity or plurality of SlyB nanodiscs may be identical in composition or differ in its composition. The SlyB proteins present in the SlyB oligomers of said lipid discs may be the same or different, and may be one of the wild type or native protein sequences of SEQ ID NO:57-84 or a naturally-occurring or variant homologue with a mature domain of at least 80% identity of any one thereof, or may contain discs that differ in SlyB protein sequence of their oligomers; or alternatively may contain a mixture, between discs or even within one disc, of SlyB proteins represented by any of the described sequences of SEQ ID NO:57-84 or homologue with a mature domain of at least 80% identity of any one thereof, and a variant or mutant form thereof. In a specific embodiment, the isolated SlyB nanodisc particle as described herein has a diameter below 40 nm, more specifically below 20 nm, even more specifically between 9 and 15 nm.

In a specific embodiment, said SlyB nanodiscs are isolated to obtain a composition, which may comprise further host-derived compounds, or further exogenously added compounds. Said compounds may be nucleic acid molecules, oligo or polysaccharides, proteins or complexes, or even chemically synthesized compounds. Another specific embodiment further relates to said SlyB lipid nanodiscs, wherein the outer saccharolipid moiety contains lipid A, a lipooligosaccharide (LOS), a lipopolysaccharide (LPS), or a modified LPS molecule, or a combination thereof, and preferably present in said SlyB oligomeric disc in a ratio of 1:1 per SlyB monomer.

A further embodiment discloses the SlyB nanodisc as described herein, further comprising a macromolecule, wherein said macromolecule is preferably encapsulated within the lipid bilayer nanodomain, thus within the lumen of the SlyB oligomeric belt. Said macromolecule may originate from the native host from which the SlyB nanodisc has been isolated, preferably a Gram-negative bacterial host, or may be a recombinantly produced macromolecule enclosed in the SlyB nanodisc upon production of the SlyB nanodomain in the host. More preferably, said macromolecule may be an endogenous or heterologous protein, such as a bacterial outer membrane protein, or a complex or more specifically a protein complex; or alternatively, may be an exogenously added nucleic acid molecule, optionally fused to a membrane protein located in said luminal lipid bilayer nanodomain. In one embodiment, the macromolecule(s) enclosed within the disc(s) are preferably not in direct contact or bound to the SlyB oligomer, but separated from SlyB within the central portion of the nanodisc by the phospholipid layer(s). In a specific embodiment, proteins present in the SlyB nanodisc are not fusion proteins, and preferably SlyB oligomers are not fused to further proteins larger than 15 kDa since this may not be beneficial for forming the discoidal belt for the nanodiscs. In a specific embodiment, said SlyB lipid nanodisc composition has a native or heterologous macromolecule present within the lumen of the nanodisc(s) which is a membrane protein, preferably an outer membrane protein, an outer membrane lipoprotein, or a β-barrel-containing membrane protein. Said macromolecule may thus be captured within said SlyB nanodisc, though still capable of displaying a portion of its surface through the lipid layers to the exterior part of the disc. So, in a further specific embodiment said centrally present macromolecule(s) within the lipid nanodisc(s) of the SlyB nanodisc composition may function as an immunogen or antigen upon administering the lipid disc composition to a subject. A further specific embodiment thus describes said isolated SlyB nanodisc composition wherein said nanodisc contains one or more macromolecules which are derived from, or at least the sequence or structure of the molecule is originating from a pathogenic species, such as a virus, a bacterium, specifically a Gram-negative bacterium, a fungus, a protozoan, a parasite, a human neoplastic cell or an animal neoplastic, tumor or cancer cell. In a more specific embodiment, the size of the SlyB nanodisc composition including the macromolecule or macromolecular complex is between 150 and 1000 kDa, more preferably between 250 and 750 kDa.

In a further embodiment, the invention relates to an immunogenic composition which contains the isolated SlyB nanodisc as described herein. Said SlyB nanodisc may be immunogenic by the presence of the discoidal SlyB belt and its (saccharo-)lipidic surrounding within the nanodisc as adjuvanting compound, so may be present in said immunogenic composition as self-adjuvanting carrier of an immunogen or antigen, represented by the macromolecule(s) preferably present within the SlyB nanodisc(s). Alternatively, the SlyB nanodisc comprised within said immunogenic composition may be functioning as a vehicle for a further component.

Another aspect of the invention relates to a chimeric gene for expression of SlyB to recombinantly produce SlyB lipid nanodiscs in a host, wherein the chimeric gene is a nucleic acid molecule which contains a promoter to induce the expression of an operably linked coding sequence for a SlyB protein comprising SEQ ID NO:57-84 or a bacterial homologue with a mature domain sequence of at least 80% identity of any one thereof, or a mutant variant of any one thereof. Preferably the chimeric gene contains a promoter that is heterologous to the SlyB coding sequence and is an inducible promoter, such as a synthetic promoter. In a specific embodiment, the chimeric gene comprises an inducible promoter as to induce SlyB expression in a host resulting in over-expression or ectopically expressed SlyB in said host, thereby providing for a SlyB-nanodisc inducing condition. Further embodiments relate to a host cell, which produces or comprises the SlyB nanodomain including the nanodisc as described herein, potentially in the form of an immunogenic composition as described herein, or alternatively a host cell containing the chimeric gene described herein, or a vector containing the chimeric gene described herein, for expressing and induction of SlyB, as to obtain a recombinant host cell with recombinantly produced SlyB forming the SlyB nanodomains and/or nanodiscs, optionally wherein the host cell is deficient in endogenous or native SlyB production.

Another aspect of the invention relates to a method to induce, produce and isolate a SlyB nanodisc in a host cell comprising the steps of: inducing the expression of recombinantly introduced SlyB in a host cell, wherein the induction results in SlyB oligomeric nanodomain formation in the membrane of the host cell, followed by purifying the SlyB nanodisc particles from said cell, preferably involving detergent-mediated extraction of detergent-soluble SlyB nanodisc-containing membrane structures, and isolating the SlyB nanodisc particles of below 40 nm diameter, preferably below 20 nm diameter, by using a size-separation technique such as gel- or ultrafiltration technique with a cut-off for separation of those small nanodiscs.

A specific embodiment relates to said method to produce a SlyB nanodisc, wherein the expressed SlyB protein is a native or wild type SlyB protein, preferably recombinantly expressed under SlyB-nanodomain-inducing conditions, as described herein, in a host cell that is lacking its native functional SlyB expression, and preferably isolated by detergent-mediated extraction.

Another specific embodiment relates to said method to produce SlyB nanodiscs, wherein the expressed SlyB protein is a tagged or a mutant or variant SlyB protein, recombinantly expressed under SlyB nanodomain-inducing conditions in a host cell, wherein said host does or does not contain a native or endogenous SlyB-encoding gene. Said SlyB deficient host may be a Gram-negative SlyB-knock-out/knock-down/or mutant bacterial host or a bacterial host lacking an endogenous SlyB-encoding gene or PhoPQ operon. Another specific embodiment relates to said method to produce a SlyB nanodisc composition, wherein the expressed SlyB protein is a recombinantly expressed SlyB comprising SEQ ID NO:1-28 or comprising any one of SEQ ID NOs: 54-87, or a bacterial homologue with a mature domain of at least 80% identity of any one of SEQ ID NOs: 57-84, or a mutant variant of any one thereof introduced in a host cell, wherein said host cell may be a bacterial host cell, a prokaryotic, fungal, or eukaryotic host cell. Another specific embodiment relates to said method to produce a SlyB nanodisc, wherein the expressed SlyB protein is expressed by introducing the chimeric gene as described herein in a host cell, and inducing the promoter of said chimeric gene, as to obtain overexpression of recombinant SlyB in said host cell.

Another specific embodiment relates to said method to produce a SlyB nanodisc composition, wherein the promoter to induce recombinantly introduced SlyB protein is a SlyB promoter and wherein the ‘SlyB-inducing conditions’, or as used interchangeably herein ‘SlyB nanodomain-inducing conditions’, comprise any of the cell culturing or incubation conditions that induce the PhoPQ stress regulon, or that apply damage to the outer membrane, which in its turn induce the SlyB promoter. Specifically said SlyB nanodomain-inducing conditions include, in a non-limiting manner, reducing the cell culture pH to 5 or lower, or addition of cationic antimicrobial peptides to the extent that the PhoP-Q regulon is induced, or depletion of divalent cations in said host cells, preferably by adding a metal chelator, such as EDTA, to said cell culture, or addition of a SlyB agonist to said cell culture for a period in time. In a further embodiment, said method to produce a SlyB nanodisc composition provides for ‘SlyB nanodomain-inducing conditions’, by inducing LPS destabilization to the cell culture, such as treating the cells with an LPS synthesis inhibitor, which is added to the extent that SlyB lipid discs are induced and formed without or with limited, altered or modified saccharolipid moieties.

In a further embodiment, said method to produce a SlyB nanodisc composition, provides for ‘SlyB nanodomain-inducing conditions’, by overexpression of a membrane protein in said host cell, more specifically an outer membrane protein, or by recombinantly expressing SlyB protein as such that the SlyB promoter operably linked to said SlyB protein encoding nucleic acid sequence in said host cell is specifically activated or triggered for a certain period of time during cell culturing, as to allow overexpression of SlyB in said cell culture. In a specific embodiment, said SlyB promoter is an endogenous or wild type SlyB promoter that is operably linked to the SlyB protein in the chimeric gene described herein. Said SlyB promoter may be induced by inducing the PhoPQ stress regulon cascade, or specifically by any of the SlyB nanodomain-inducing conditions described herein. In a further specific embodiment, said SlyB promoter is a heterologous promoter operably linked to the SlyB protein-encoding sequence of said chimeric construct described herein, wherein said heterologous promoter is an inducible promoter to allow (temporary) overexpression of SlyB in said host cell, which induces SlyB oligomer formation. Preferably said inducible promoter is a bacterial and/or synthetic promoter.

Another specific embodiment relates to said method for producing and isolating SlyB nanodisc particles comprising the steps of:
inducing expression of recombinant SlyB in a host cell, wherein the induction specifically results in SlyB oligomer formation, as described for the method above, and
purifying the SlyB nanodisc particles from said cell, preferably involving detergent-mediated extraction and size-separation of SlyB nanodisc-containing membrane structures, wherein said isolated SlyB optionally comprises a further macromolecule and/or host cell protein or complex,
and optionally, a further step of combining said purified SlyB nanodisc particles with another macromolecule to enclose within said SlyB nanodiscs of said composition.

A further embodiment relates to said method to produce a SlyB nanodisc composition, wherein the host cell additionally comprises an exogenous macromolecule that is not SlyB, wherein said macromolecule may be a protein, an oligo- or polysaccharide, a nucleic acid molecule, or a complex of any one thereof, which is upon expression of SlyB encapsulated in said SlyB nanodisc.

A further related aspect of the invention relates to the SlyB nanodisc composition obtainable by any of the methods to produce said SlyB nanodisc described herein.

In a final aspect, the SlyB nanodisc or immunogenic composition comprising said SlyB nanodisc as described herein may be used for immunization of a subject. In a specific embodiment, the SlyB nanodisc acts as a self-adjuvanting vehicle when used for immunization and/or eliciting a humoral immune response. Thus SlyB nanodiscs as described herein may be used as a vaccine or in a vaccine composition. Further embodiments thus relate to the use of SlyB nanodiscs in presenting an antigen or macromolecule, or alternatively in presenting a membrane protein or macromolecule enclosed within the lumen of the nanodisc, usable in different applications wherein membrane proteins are required in a stable position, or need to be inserted in lipid environments.

### DESCRIPTION OF THE FIGURES

The patent or application file contains at least one drawing executed in color. Copies of this patent or patent application publication with color drawing(s) will be provided by the Office upon request and payment of the necessary fee.

The drawings described are only schematic and are non-limiting. In the drawings, the size of some of the elements may be exaggerated and not drawn on scale for illustrative purposes.

FIGS. 1A-1D. SlyB is essential under PhoP/Q inducing OM stress conditions.

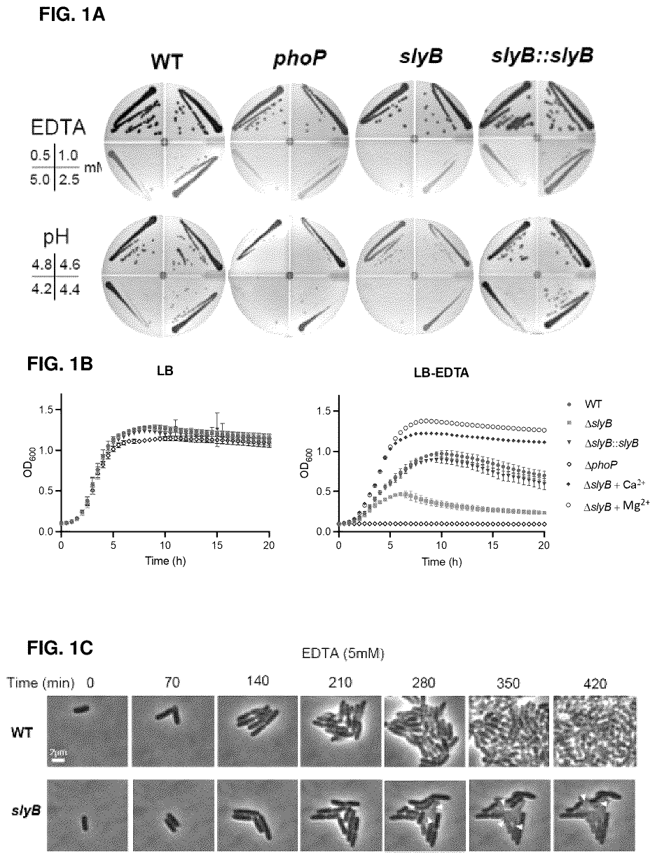

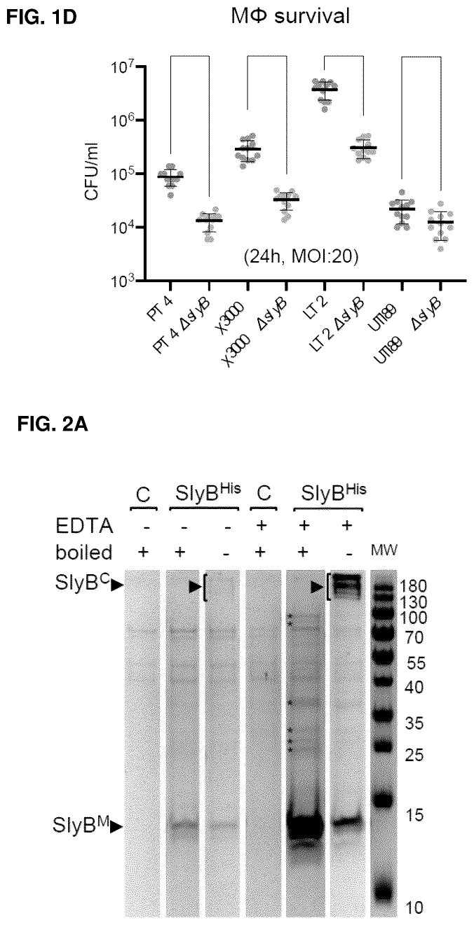

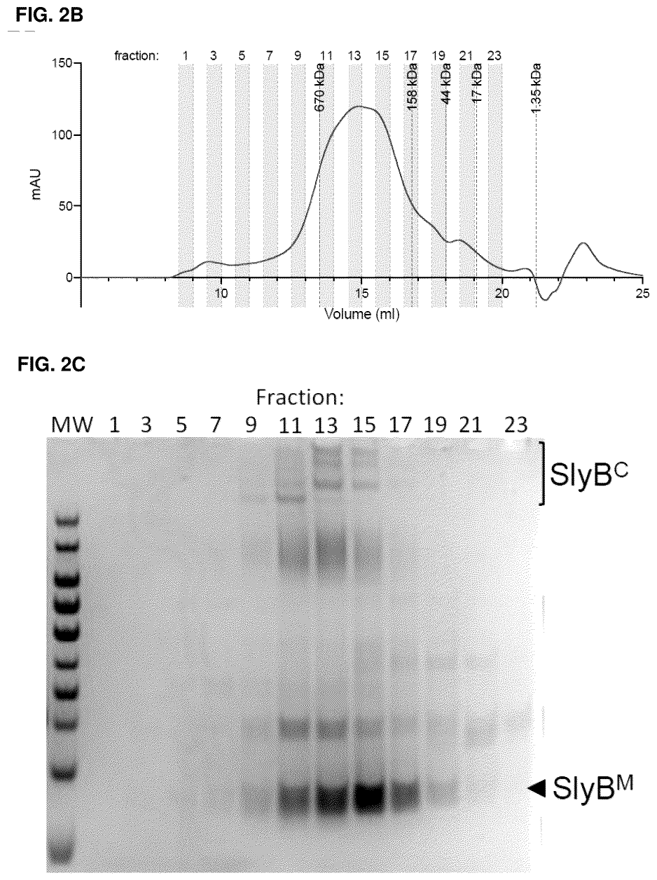

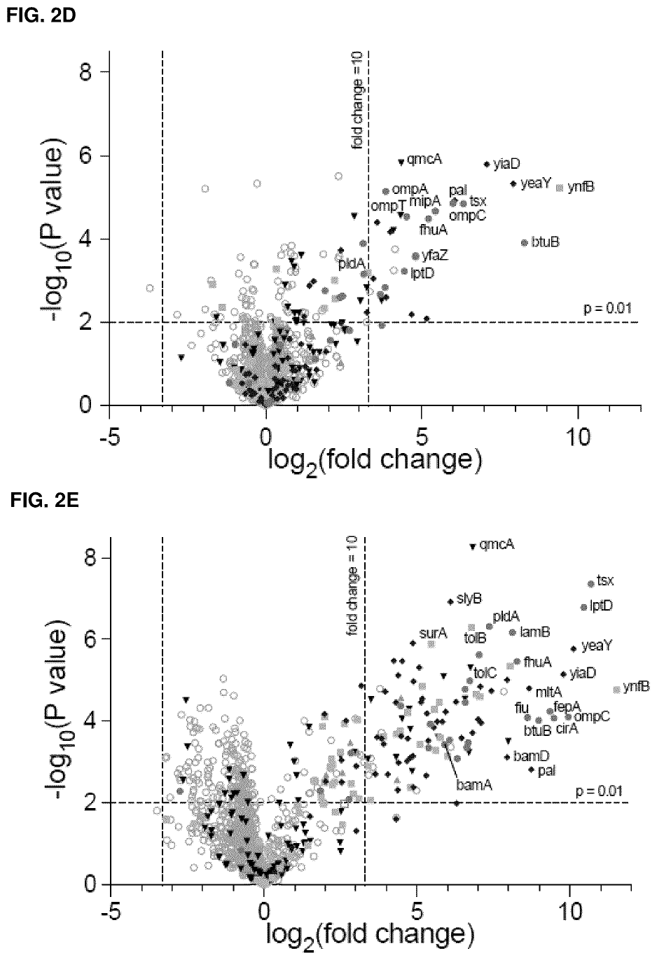

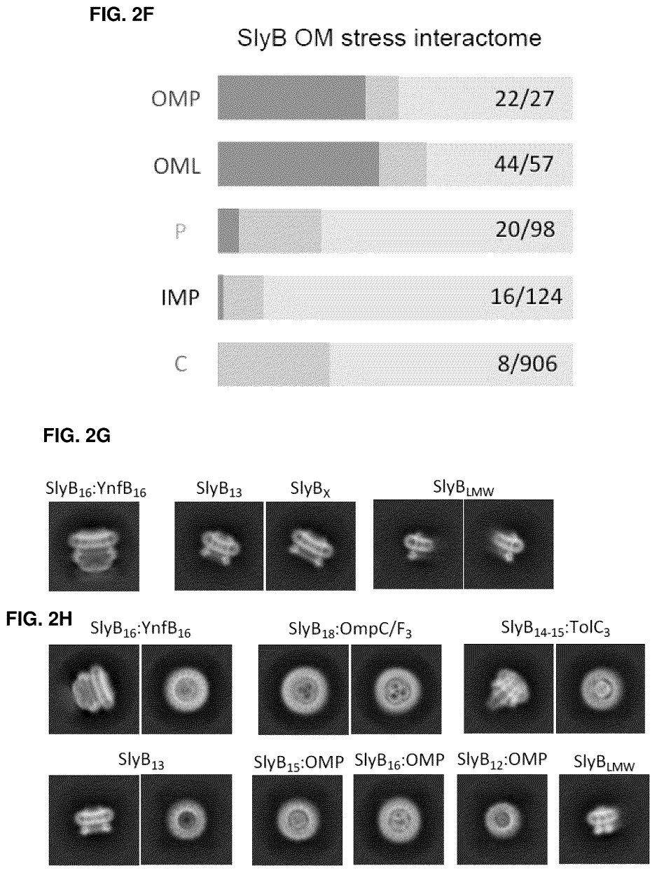

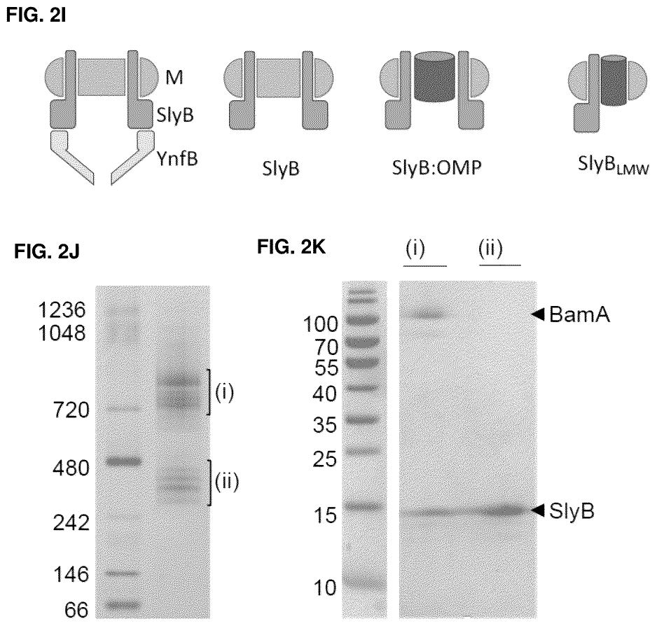

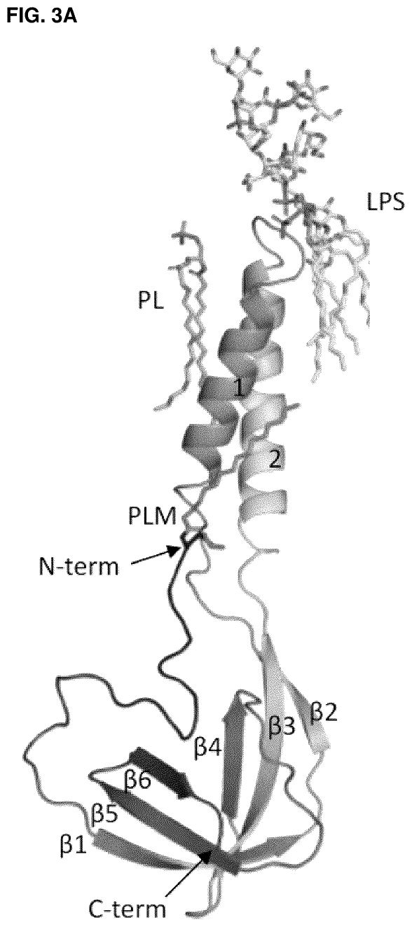

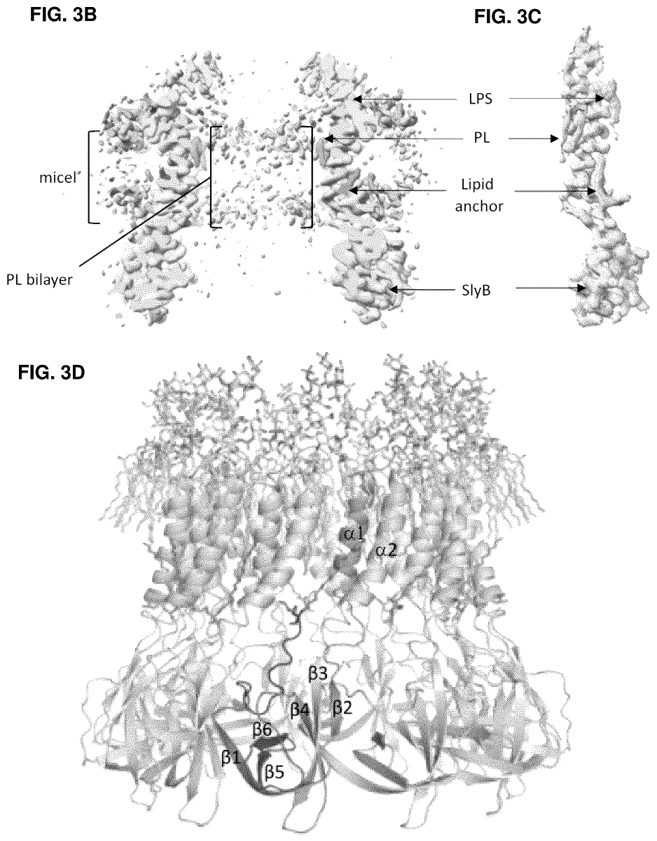

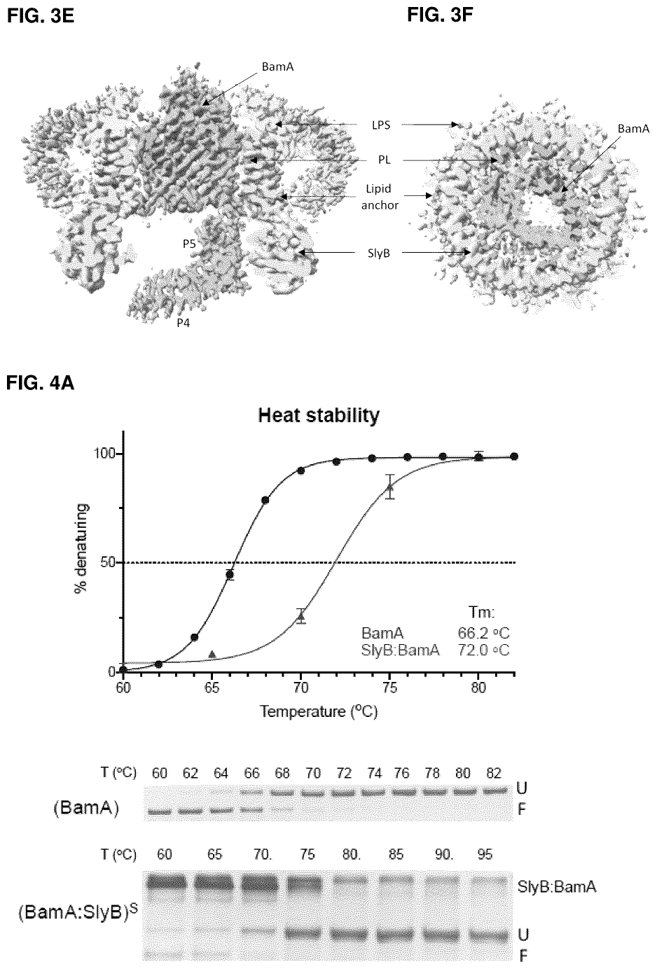

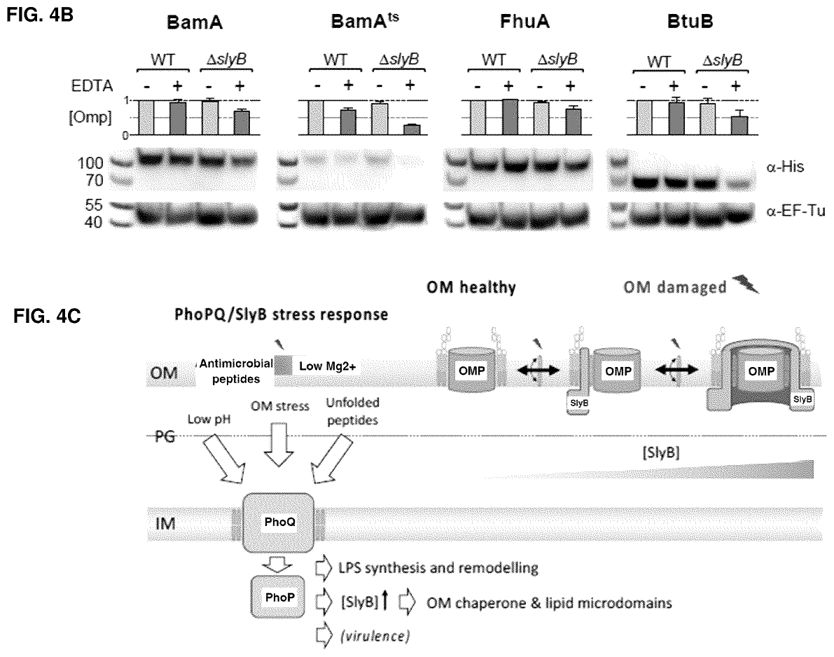

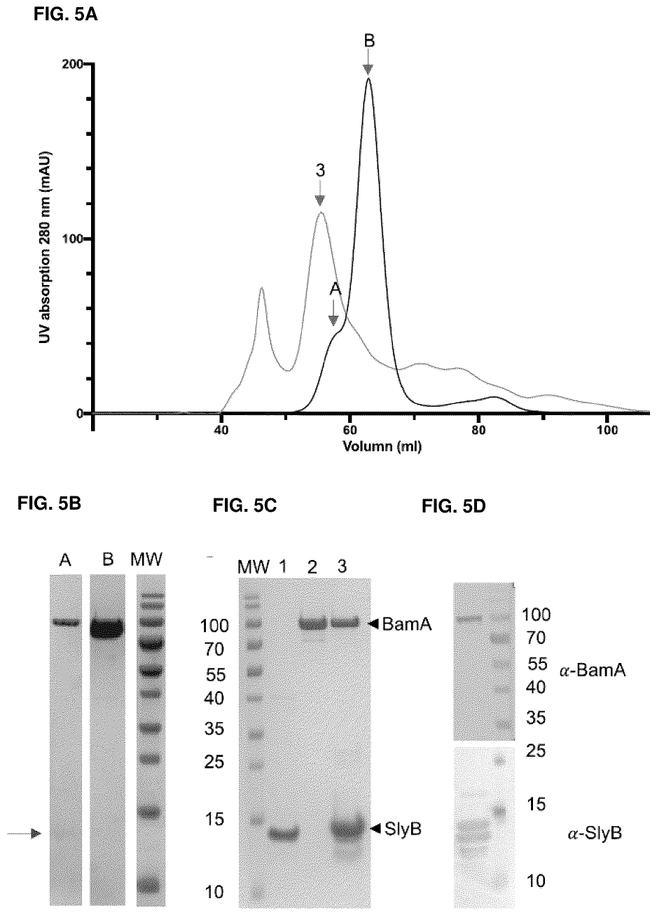

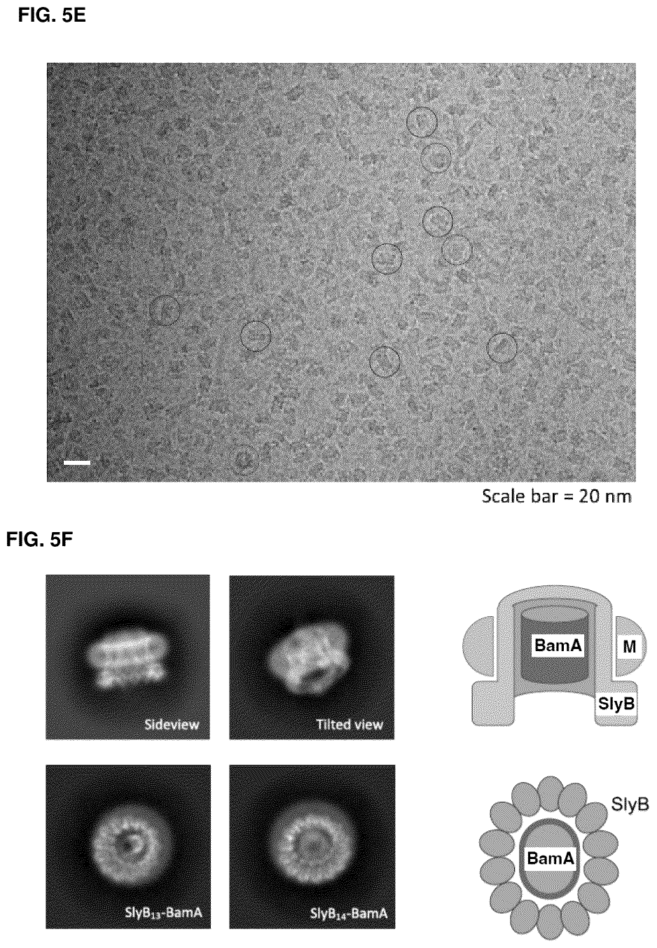

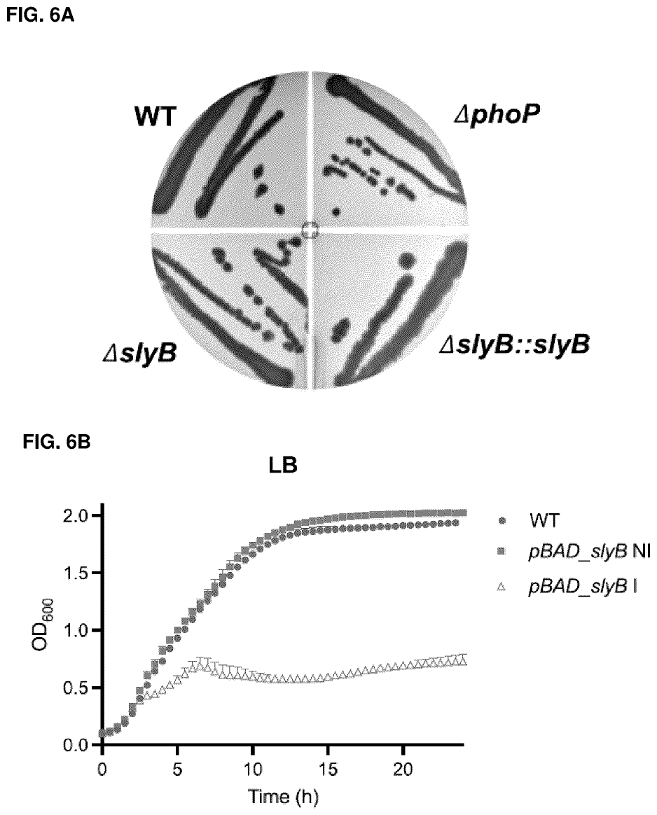

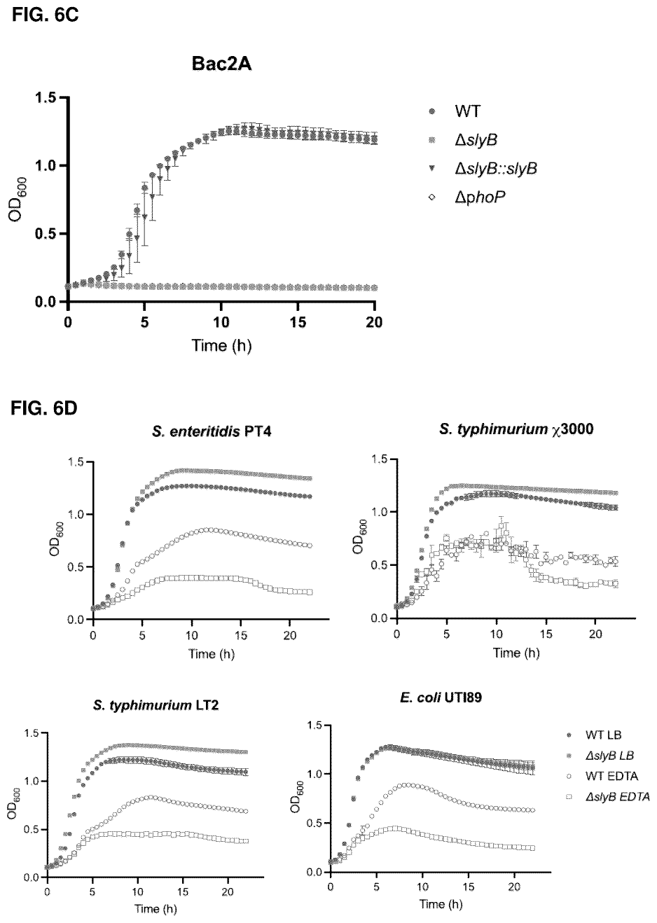

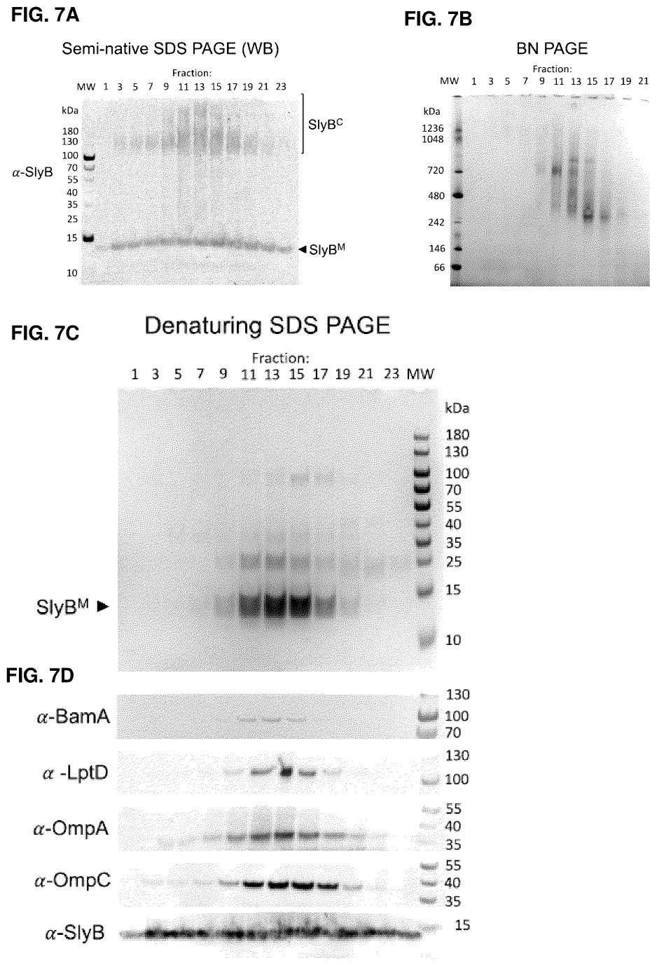

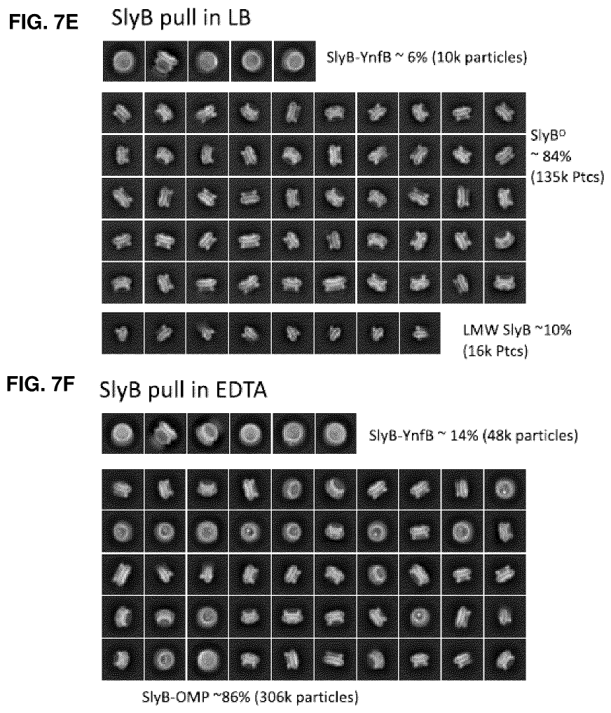

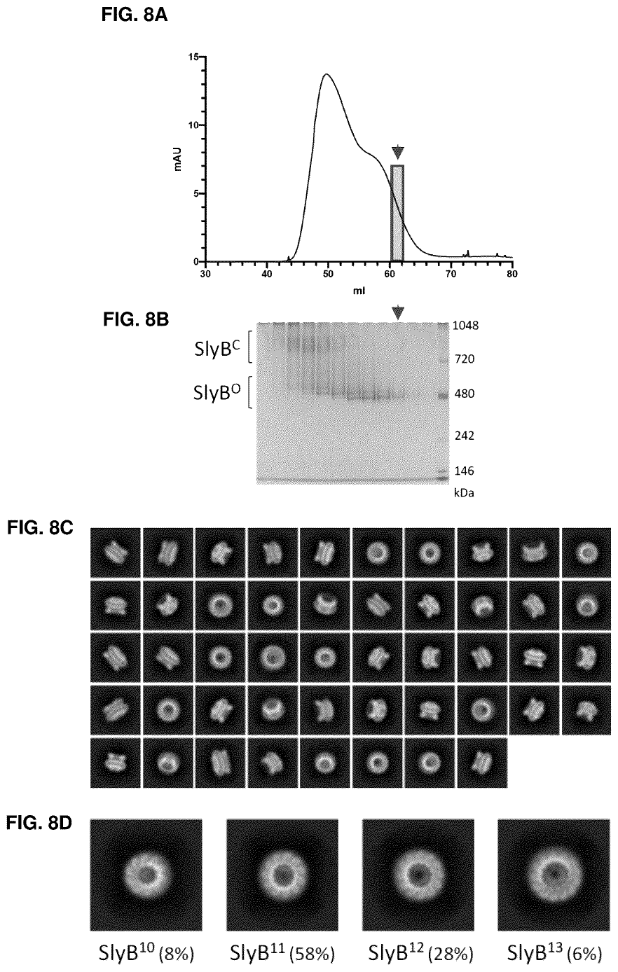

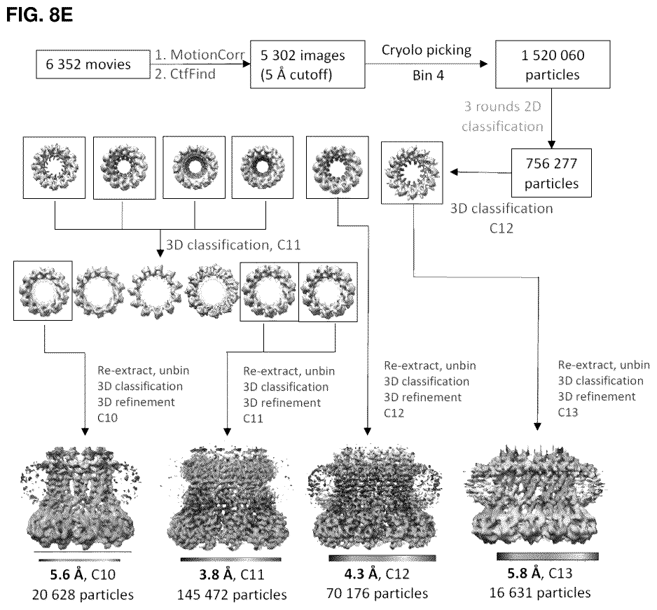

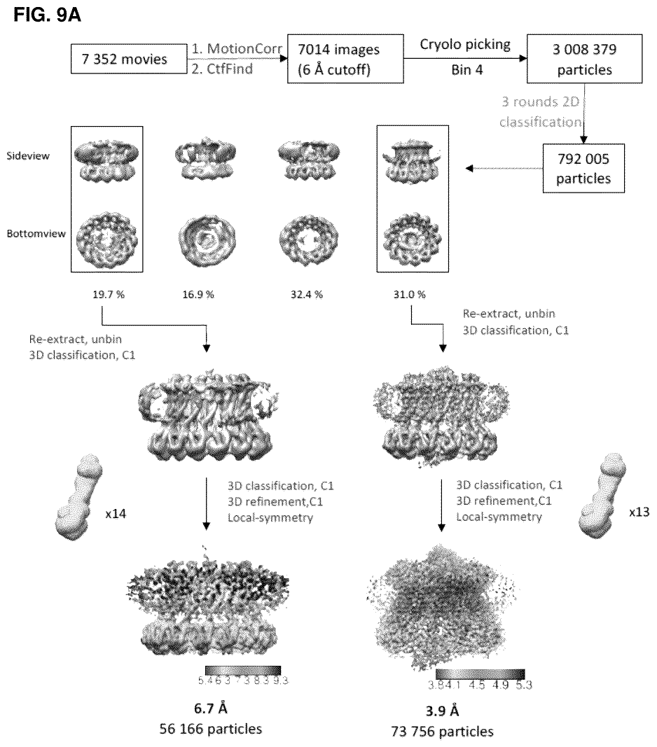

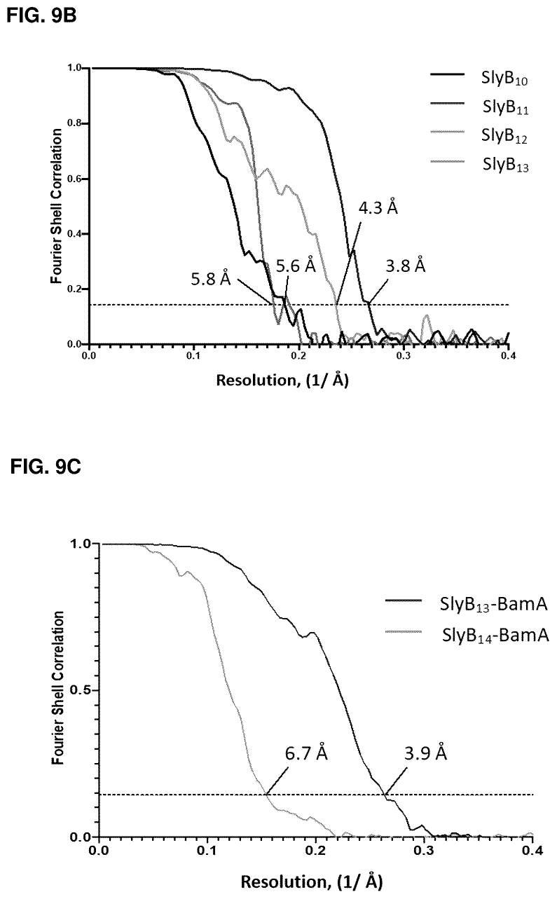

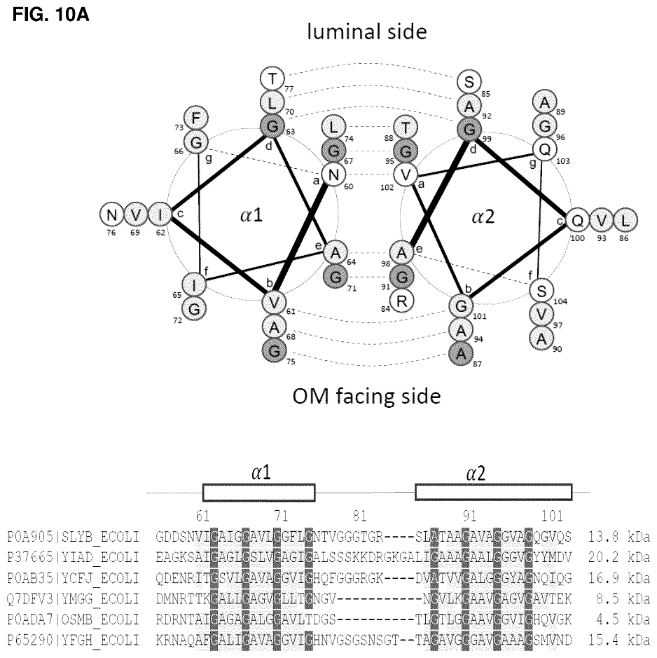

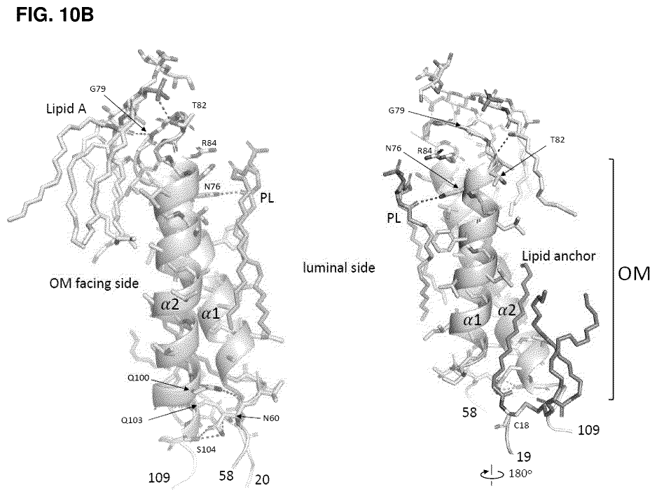

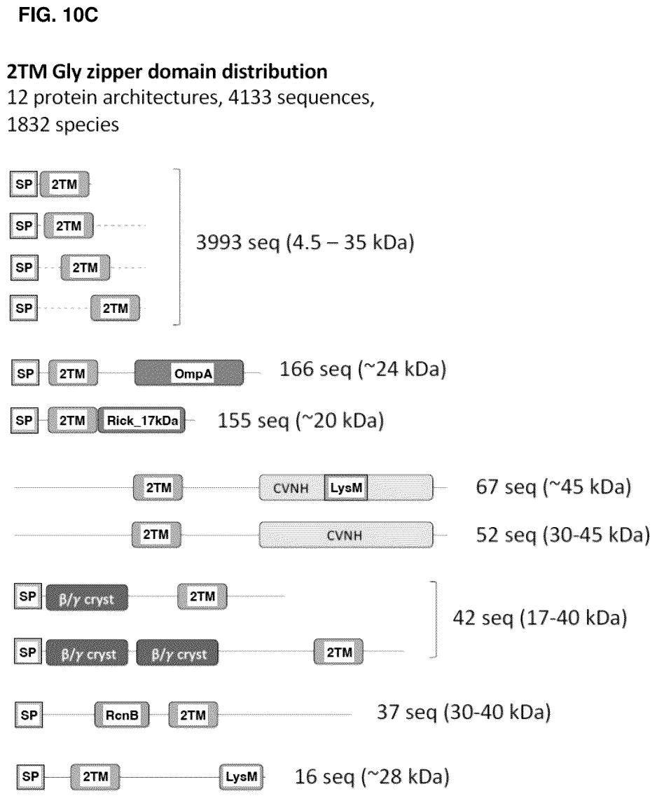

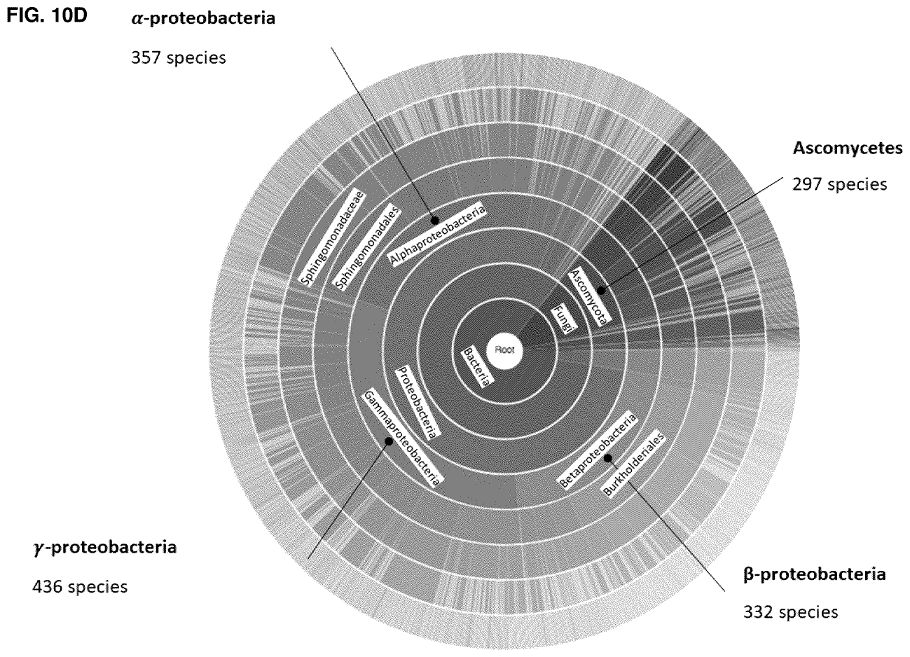

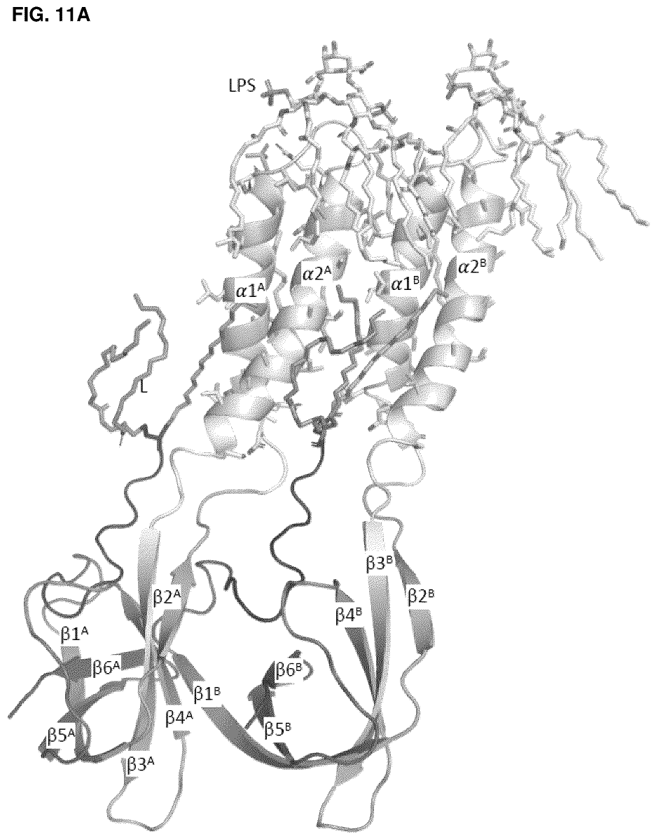

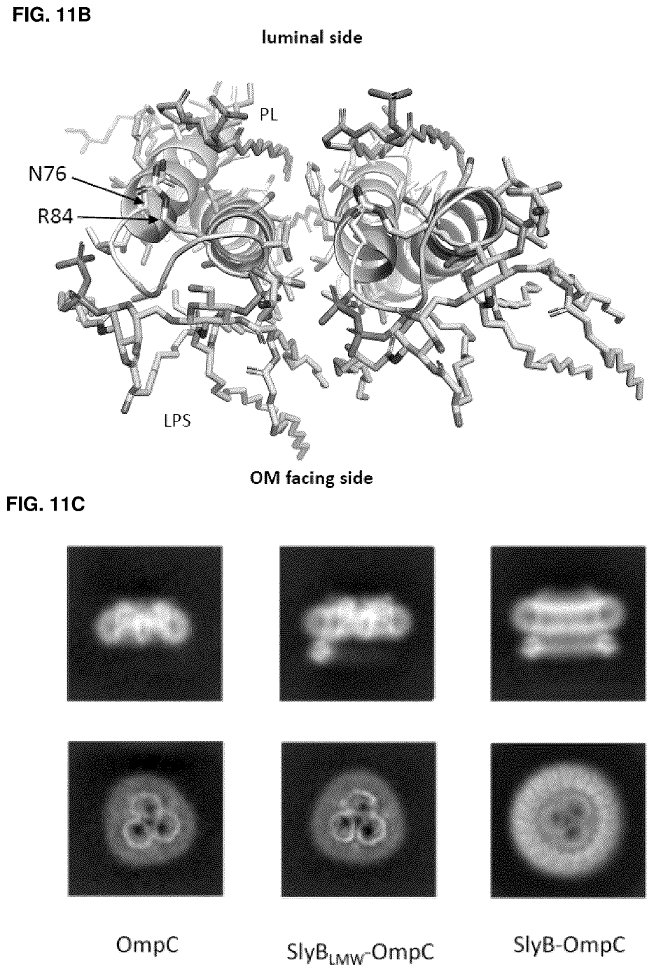

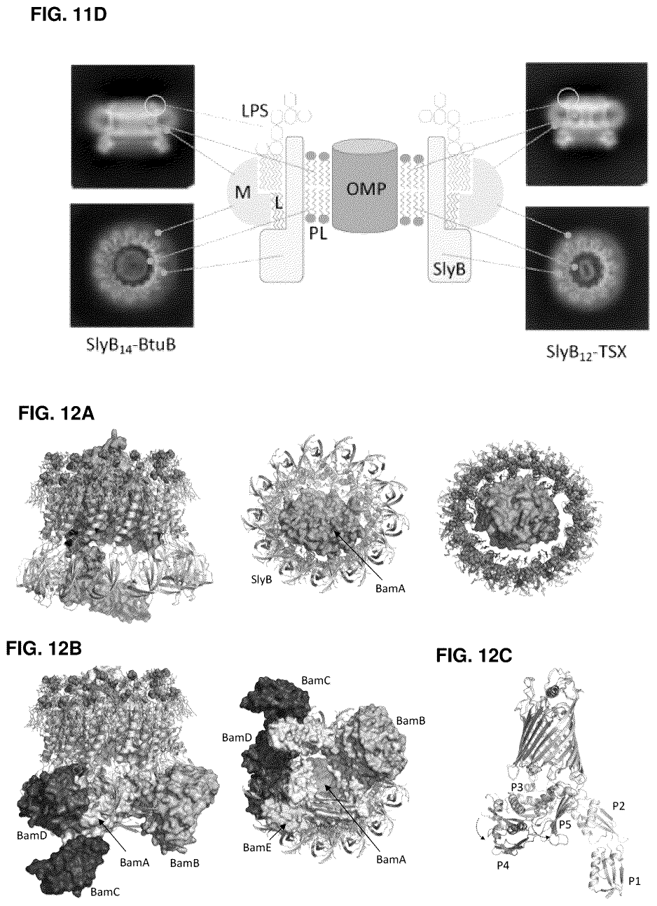

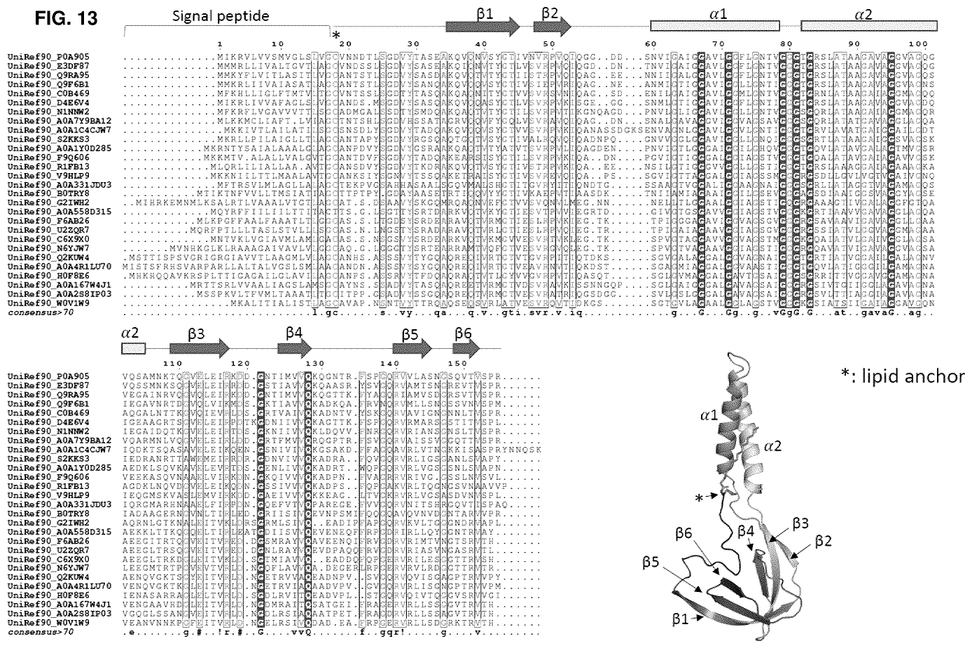

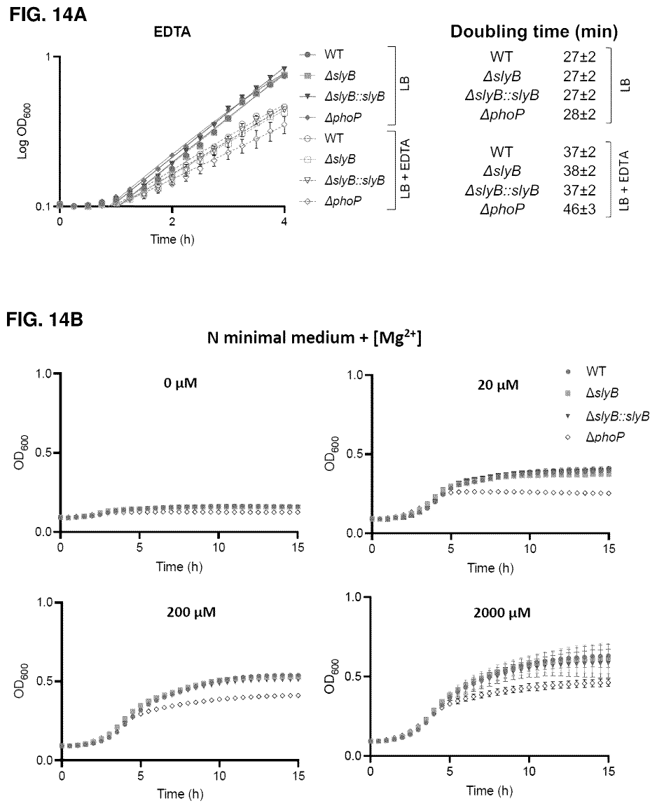

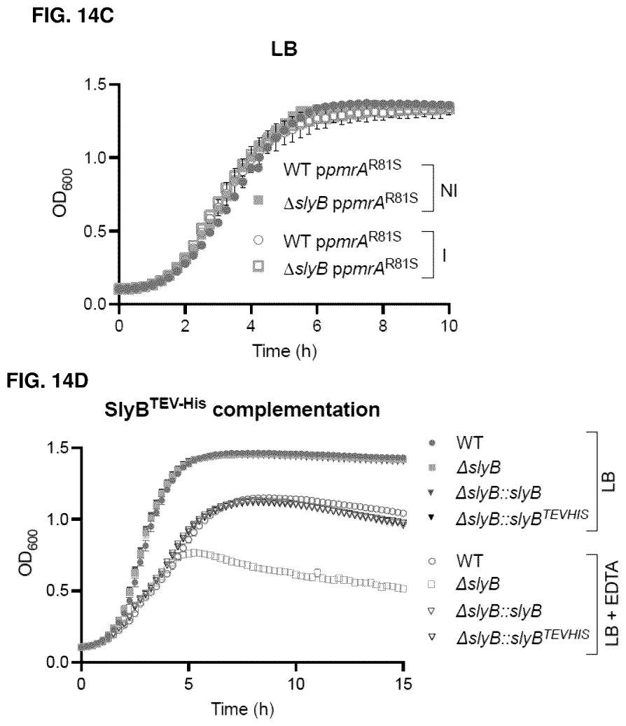

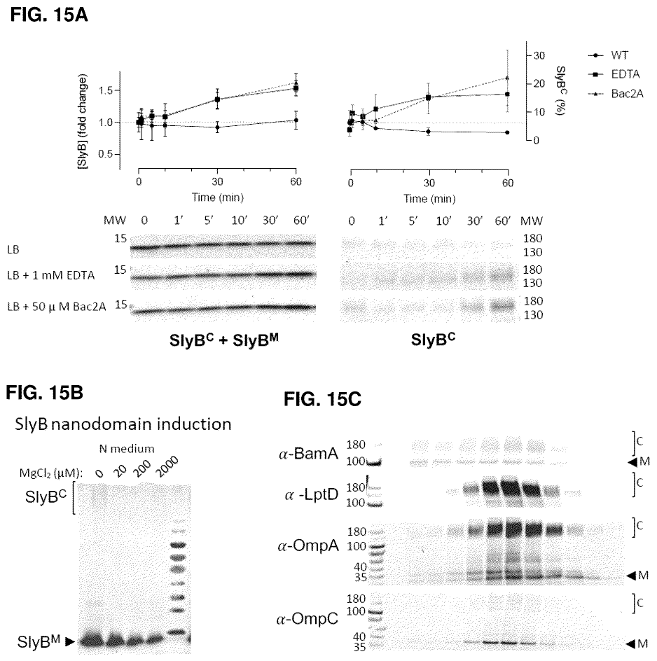

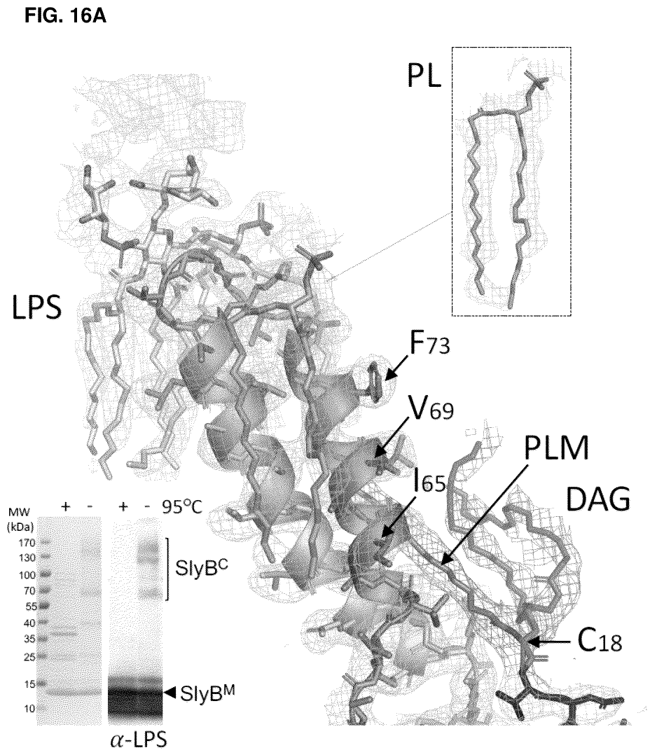

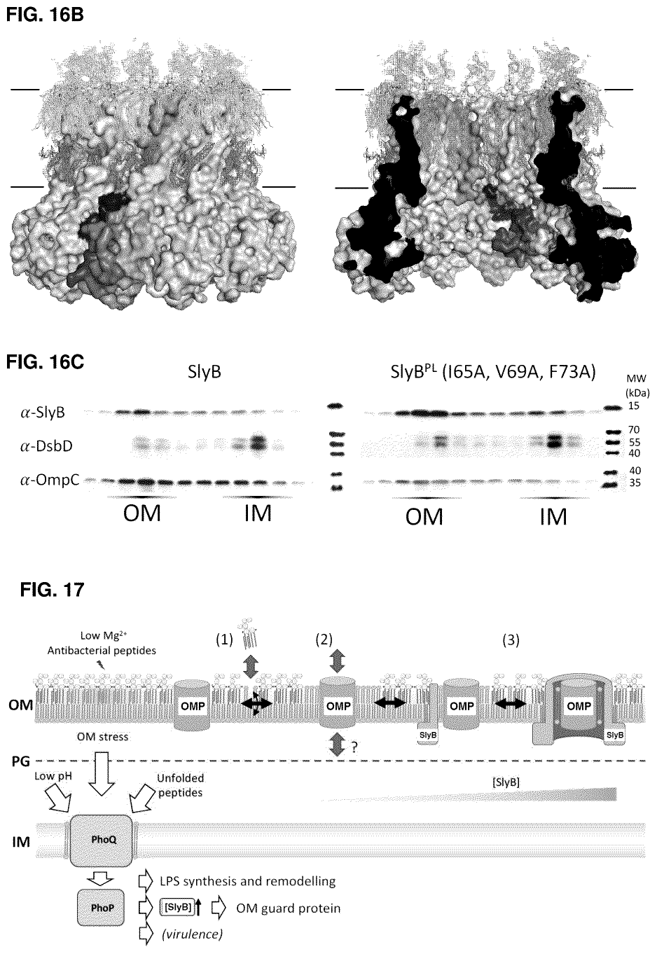

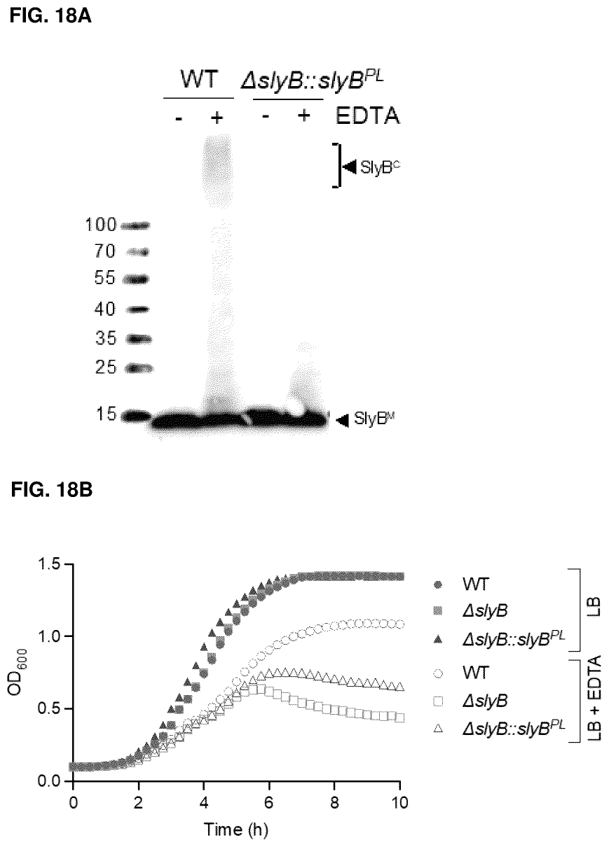

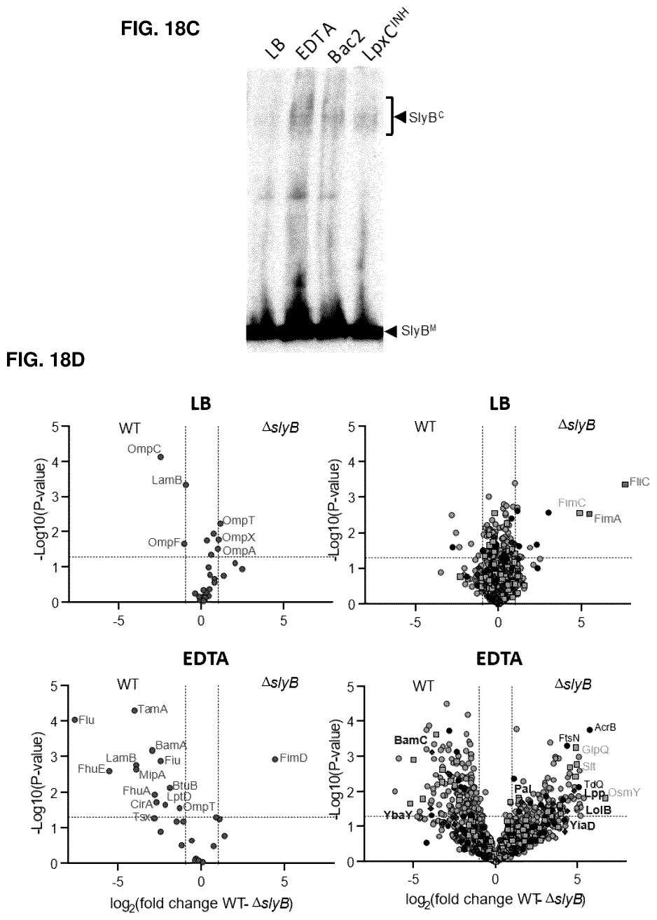

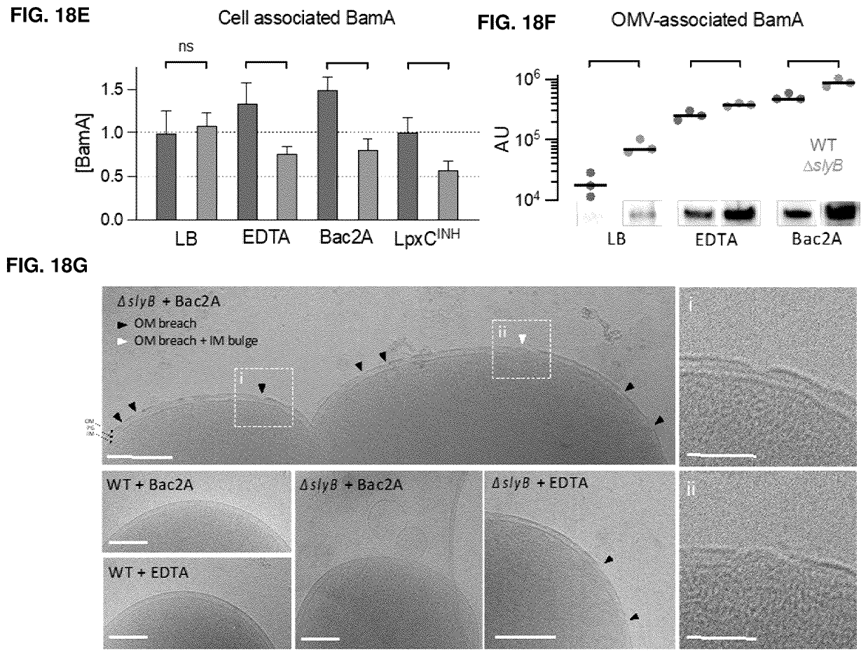

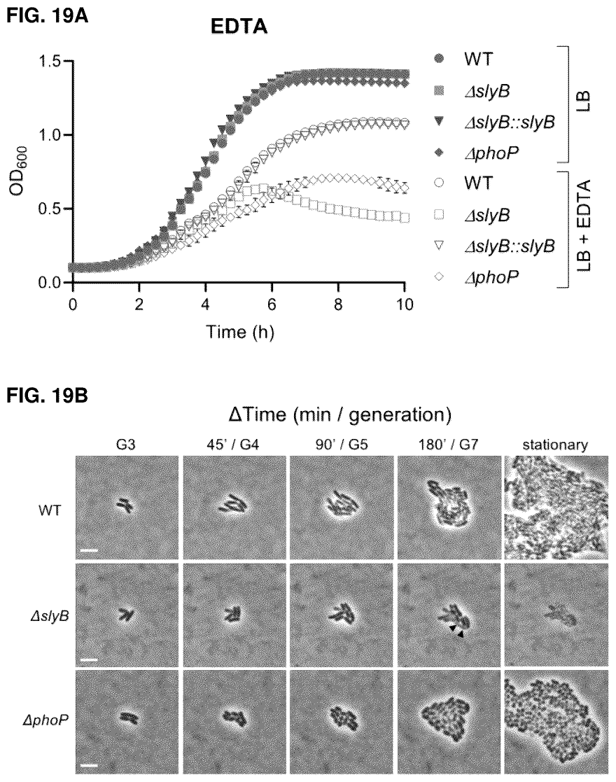

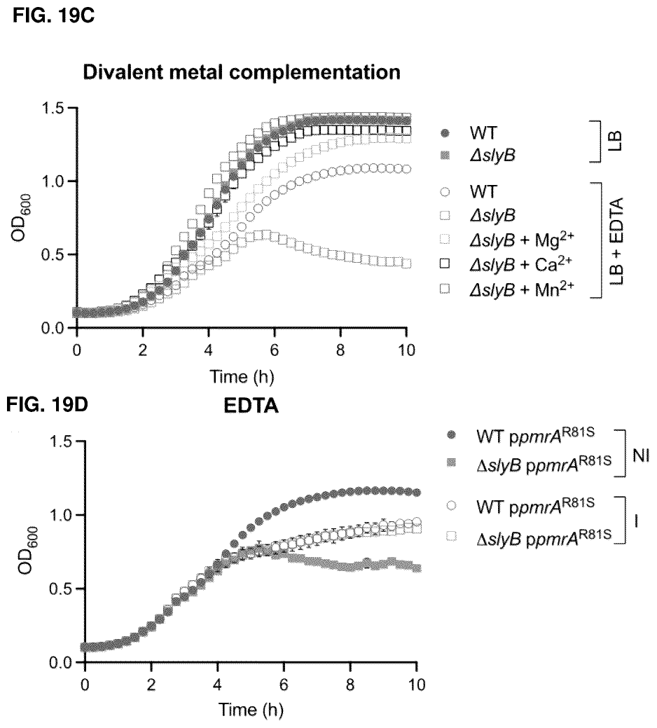

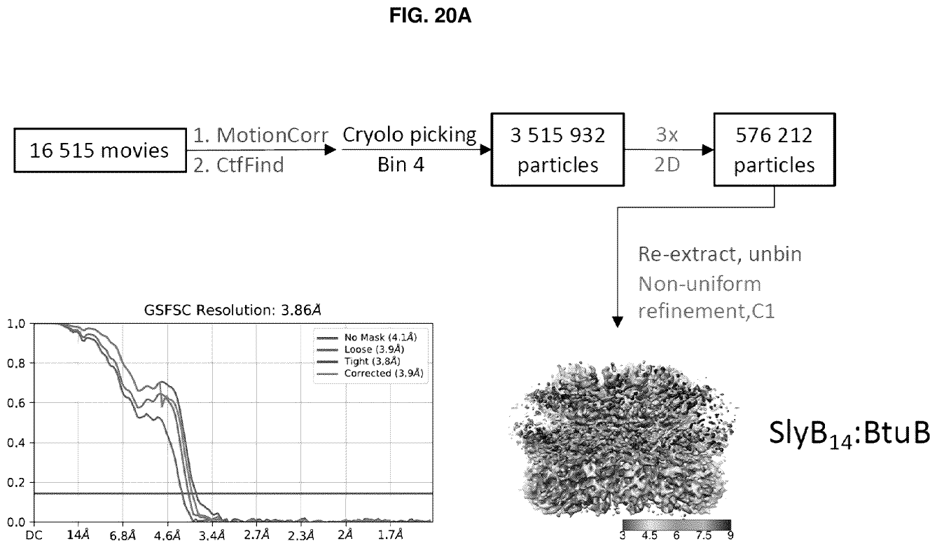

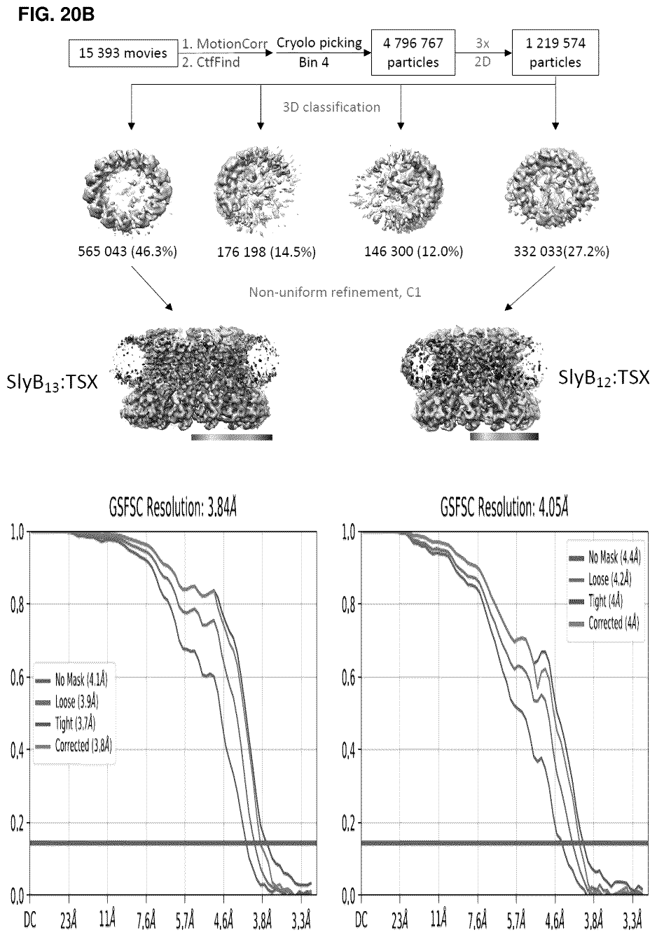

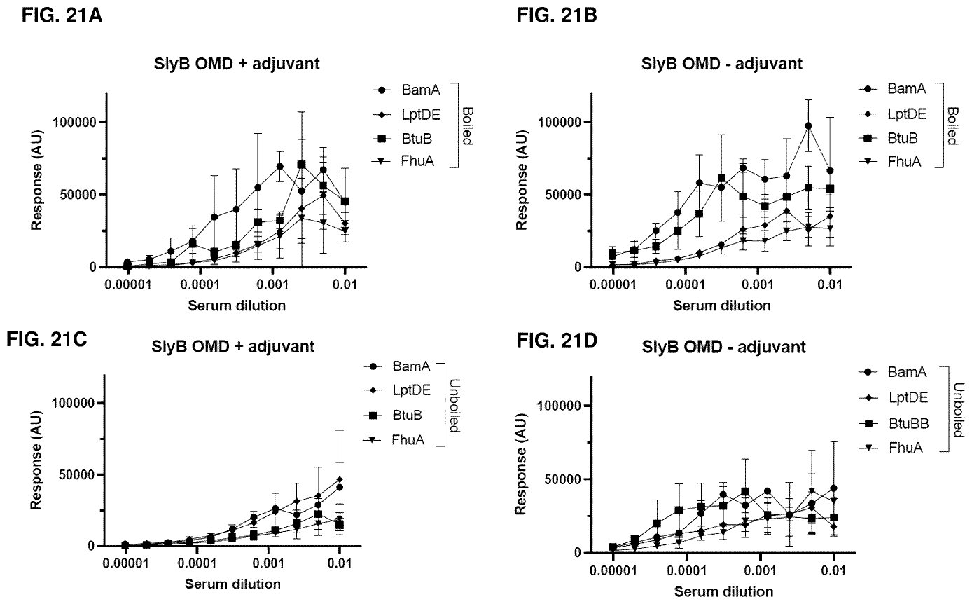

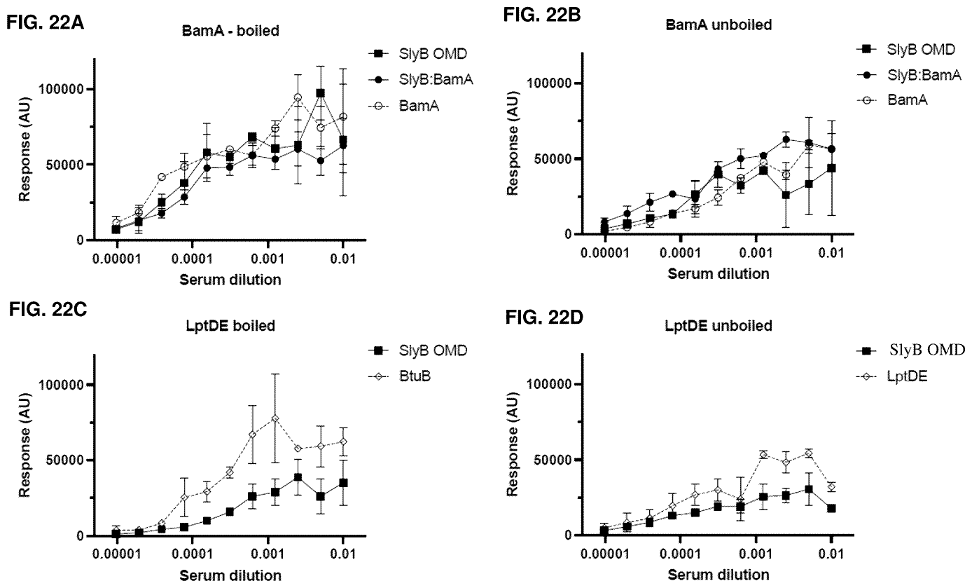

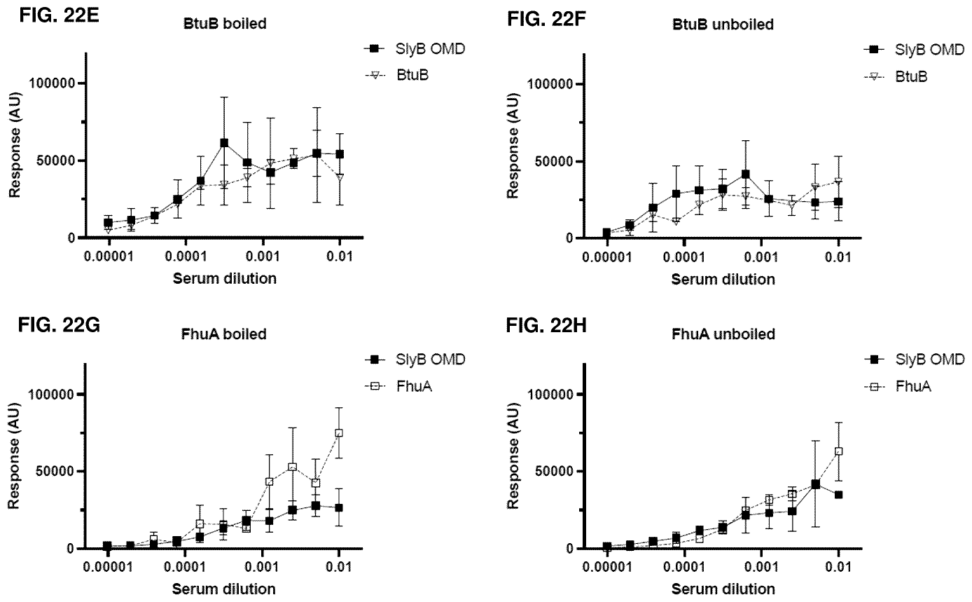

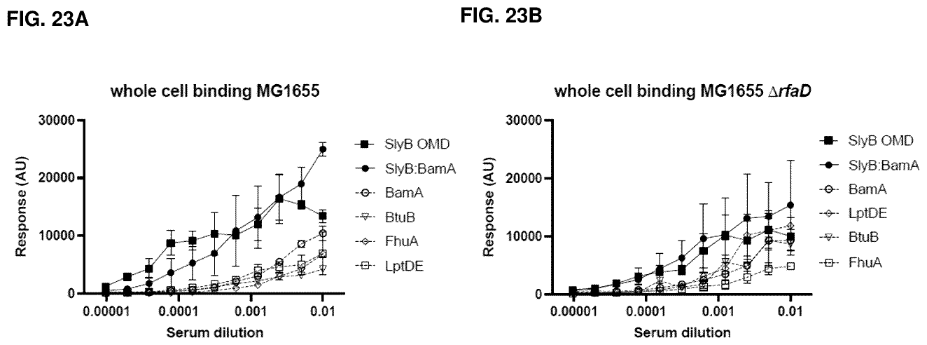

(FIG. 1A E. coli BW25113 and derivative strains ΔslyB, ΔphoP and ΔslyB::slyB streaked on sectional LB-agar plates supplemented with varying concentrations of known PhoP/Q inducers. (FIG. 1B) OD₆₀₀ growth curves of E. coli BW25113 and derivative strains ΔslyB, ΔphoP and ΔslyB::slyB in LB (left panel) or LB supplemented with 5 mM EDTA (right panel). Conditions labeled ‘+Mg²⁺’ or ‘+Ca²⁺’ represent media complemented with 10 mM of the respective ion. Mean±SD (n=4 biological replicates). (FIG. 1C Time lapse phase-contrast imaging of BW25113 and BW25113 ΔslyB grown on LB agar with 5 mM EDTA. White arrows indicate lysed cells. Data representative of at least three biological replicates. (FIG. 1D) Intramacrophage survival and replication of clinical Salmonella enterica (PT4, χ3000 and LT2) and E. coli (UTI89) isolates and their ΔslyB derivatives. 5*10⁴ cells/ml of macrophages were infected with bacteria at an MOI of 20. Titers of surviving bacteria after overnight infection drastically decreased for ΔslyB mutants of each tested strain, compared to WT titers. Mean±SD (n=12; unpaired T-test, ****: p<0.001, *: p<0.05).

FIGS. 2A-2K. SlyB binds the OM proteome under stress.

(FIG. 2A) Semi-native PAGE of Ni-NTA pulldowns from BW25113 (C) or BW25113 ΔslyB::slyB^{TEV_His} (labelled SlyB^{His}) grown in LB medium with (+) or without (−) 5 mM EDTA. Bands corresponding to high MW SlyB complexes or monomeric SlyB are labeled SlyB^{C} and SlyB^{M}, resp. Asterisks in the boiled sample indicate SlyB partners released from SlyB^{C}. (FIG. 2B) SEC profile and fractionated semi-native PAGE (FIG. 2C) of Ni-NTA pulldowns from BW25113 ΔslyB::slyB^{TEV_His} grown in LB+EDTA medium. Elution volumes of MW standards indicated by vertical lines. (FIGS. 2D and 2E) Volcano plots of the SlyB interactome identified by peptide mass fingerprint from Ni-NTA pulldowns from BW25113 ΔslyB::slyB^{TEV_His} in LB (d) and LB+EDTA (e) conditions. Proteins are colored according to subcellular localization: Outer membrane protein (OMP;red), Outer membrane Lipoprotein (OML; blue), periplasmic (P; orange), Inner membrane protein (IMP;black) and cytoplasmic (C; purple). (FIG. 2F) Fraction of SlyB interactors according to subcellular localization (light red; >10-fold enriched and p<0.01) relative to total detected (dark gray) and total genome (light grey). (FIGS. 2G and 2H) Selected cryoEM 2D classes of particles observed in Ni-NTA pulldowns from BW25113 ΔslyB::slyB^{TEV_His} in LB (g) and LB+EDTA (h). Box size: 275 A. (FIG. 2I) Schematic drawing of different SlyB complexes seen in SlyB interactome, showing SlyB (blue), YnfB (yellow), OMP (green) and detergent/lipid (orange). (FIGS. 2J and 2K) Blue-native PAGE (k) of SlyB:BamA affinity pulldown, and 2^{nd} dimension SDS-PAGE of indicated bands (i) and (ii).

FIG. 3A-3F. SlyB form lipid nanodiscs.

(FIG. 3A) Ribbon diagram of the SlyB protomer as found in SlyB₁₁ oligomer complexes (colored blue-red from N- to C-terminus). The N-terminal lipid anchor, and a bound PL and LPS molecule are shown in stick representation and colored slate, magenta and yellow, resp. (FIGS. 3B and 3C) Electron potential map of SlyB₁₁, shown as side view slice-through (b) or covering a single SlyB protomer (c). The map shows unambiguous density for SlyB (sand), its lipid anchor (slate), PL (magenta), LPS (yellow) and residual density corresponding to the detergent micelle and a luminal PL bilayer. (FIG. 3D) Ribbon diagram of the SlyB₁₁ oligomer (sand), with one protomer, the lipid anchor, PL and LPS shown and colored as in panel (a). (FIGS. 3E and 3F) Side (e) and top (f) viewed slice-throughs of the electron potential maps of the SlyB₁₃:BamA complex. Density corresponding to SlyB, lipid anchor, PL and LPS are colored as in panel (b), BamA is colored green.

FIGS. 4A-4C. SlyB protects the outer membrane proteome under stress conditions.

(FIG. 4A) Thermal unfolding plot showing % denatured BamA as monitored by semi-native SDS-PAGE (lower two panels) of BamA or SlyB:BamA complexes pretreated for 5 min at the indicated temperatures. Mean±SD (n=3). Tm: melting temperature (T at 50% denaturation). (FIG. 4B) Western analysis of OMP levels in whole cell extracts of BW25113 and BW25113 ΔslyB cells grown in LB or LB+5 mM EDTA, and expressing His-tagged BamA, the temperature sensitive mutant BamA^{ts} (i.e. R661G/D740G), FhuA or BtuB. Proteins are monitored by anti-His, using samples normalized by OD₆₀₀. Bar graphs show OMP concentration relative to WT-LB condition, measured by band density and normalized by anti-EF-Tu Western. Mean±SD (n=3). (FIG. 4C) Proposed role of SlyB in the PhoPQ regulon and OM protein homeostasis under OM stress conditions. OM damage results LPS destabilization and increased PL in the OM outer leaflet. PhoPQ activation 1) induces pathways aimed at LPS production and remodeling to restore membrane stability and asymmetry, 2) induces the overproduction of OM lipoprotein SlyB, and 3) induces virulence pathways in enteric pathogens (species variable, pleiotropic function). Under non-stressed conditions, SlyB is present as SlyB₁₆:YnfB₁₆ complexes of unknown function as well as SlyB phospholipid nanodomains (not shown). The excess SlyB produced under OM damage binds OMPs and OMLs in regions with compromised lipid asymmetry. Encapsulation of OMPs (and possibly OMLs) into SlyB bound lipid domains protects them from degradation and/or loss from the OM. OM: outer membrane; IM: inner membrane; PG: peptidoglycan.

FIGS. 5A-5F. Identification of the SlyB:BamA complex.

(FIGS. 5A and 5B) SEC profiles (a) and SDS-PAGE analysis (b) of recBamA purified from E. coli BL21AI pBamA (blue) showed the presence of BamA (B) as well as a contaminant shoulder at higher molecular weight (A). The latter fraction contained BamA as well as a ˜14 kDa protein, identified as SlyB by MS peptide fingerprinting (red arrow). Shown in green is the SEC profile of SlyB:BamA (labeled 3) purified from E. coli BL21AI pSlyB_BamA by 2-step affinity chromatography. (FIG. 5C) SDS-PAGE of purified SlyB (lane 1), BamA (lane 2), or the SlyB:BamA complex purified by 2-step affinity chromatography (i.e. lane 3). (FIG. 5D) anti-BamA and anti-SlyB stained western blot of the fraction containing the SlyB:BamA complex (lane 3). (FIG. 5E) Representative cryoEM micrograph of SlyB:BamA complex at 60K magnification. Top and side views are indicated by red and blue circles, resp. (FIG. 5F) Selected 2D cryoEM classes showing side (upper) and top (lower) views of SlyB:BamA complex, as well as the schematic presentation of the complex in both views. Box size: 275 Å. Green: BamA, blue: SlyB and orange: membrane/detergent (M). The topview reveals a SlyB oligomer comprising 13 or 14 SlyB copies that enclose a single BamA copy (SlyB₁₃-BamA and SlyB₁₄-BamA, resp.). (M: detergent micelle).

FIGS. 6A-6D. SlyB is required for growth under OM stress conditions.

(FIG. 6A) E. coli BW25113 and derivative strains ΔslyB, ΔphoP and ΔslyB::slyB streaked on sectional LB-agar plates in non-stressed condition. (FIG. 6B) OD₆₀₀ growth curves of E. coli BW25113 and BW25113 pBAD43_slyB grown in LB, without (NI) or with (I) L-arabinose induction. (FIG. 6C) OD₆₀₀ growth curves of E. coli BW25113 and derivative strains ΔslyB, ΔphoP and ΔslyB::slyB in LB supplemented with 100 μg·mL⁻¹ of the antimicrobial peptide Bactenecin 2A²¹. (FIG. 6D) OD₆₀₀ growth curves of S. enterica Sv Enteriditis strain PT4, S. enterica Sv. Typhimurium strains χ3000 and LT2, and E. coli strain UTI89 and their ΔslyB derivatives, grown in LB or LB supplemented with 5 mM EDTA.

FIGS. 7A-7F. The SlyB interactome under non stressed conditions.

(FIGS. 7A-7C) anti-BamA stained western blot of semi-native SDS-PAGE (a) and Coomassie stained blue-native PAGE (b) and denaturing SDS-PAGE (c) of Ni-NTA pulldowns from BW25113 ΔslyB::slyB^{TEV_His} grown in LB+5 mM EDTA medium. Fractions are as in FIG. 2b (see FIG. 2c for the corresponding Coomassie stained semi-native SDS PAGE). Bands corresponding to monomeric SlyB or high molecular weight SlyB complexes are labelled SlyB^{M} and SlyB^{C}, resp. (FIG. 7D) Western blot analysis of the SEC elution fractions run on denaturing SDS-PAGE (c) using anti (α)-BamA, α-LptD, α-OmpA, α-OmpC and α-SlyB as primary immune serum. MW: standards with molecular weight indicated in kDa. (FIGS. 7E and 7F) Representative 2D cryoEM classes from Ni-NTA pulldowns of BW25113 ΔslyB::slyB^{TEV_His} grown in LB (e) or LB+5 mM EDTA medium (f), generated by Cryosparc⁴². Box size: 275 Å. Both datasets contain a series of homogeneous classes corresponding to a SlyB:YnfB complex with C 16 symmetry (i.e. 16:16 stoichiometry) and corresponding to 6 and 14% of the aligned particles in the LB and LB+EDTA datasets, resp. (top row in e and f). The YnfB protein was identified as the dominant tryptic peptide in the MS peptide fingerprint and the sole significant periplasmic SlyB binding partner in the LB dataset (FIG. 2d). The remaining particles in the LB correspond to SlyB oligomers (SlyB⁰; middle five rows) of variable diameter and protomer number (as judged by top views), as well as a smaller fraction of particles corresponding to micelles with a low molecular weight SlyB complex (a single to a few SlyB protomers only; bottom row). In the LB+EDTA dataset, the majority of particles corresponds to SlyB oligomers as well as different SlyB:OMP complexes (bottom five rows).

FIGS. 8A-8E. Structure determination of SlyB oligomers.

(FIG. 8A) SEC profile (Superdex S200 16/600 column) of affinity purified SlyB. SlyB was over-expressed in E. coli BW25113 carrying pSlyB following by a 2-step Ni-IMAC purification on SlyB. (FIG. 8B) Blue-native PAGE of the elution fractions from SEC run shown in (a). Red arrow indicates the fraction that was used for cryo-EM single particle analysis. Bands corresponding to SlyB complexes or SlyB oligomers are labelled SlyB^{C} and SlyB^{O}, resp. (FIG. 8C) 2D classification analysis of SlyB^{O} single particles corresponding to the fraction indicated with the red arrow in panel a and b. Panel shows representative 2D classes generated by Cryosparc⁴². Box size: 275 Å. (FIG. 8D) Selected 2D classes showing top views of different SlyB oligomerization states (labelled SlyB^{X}, with X=the number of protomers in the particle) present in the analyzed fraction. Relative abundance of the oligomeric states indicated in parentheses. (FIG. 8E) Summary of the data processing strategies for the different SlyB oligomer reconstructions described herein. Steps performed using Relion⁵⁰ or Cryosparc⁴² are colored green or red, resp.

FIGS. 9A-9C. Structure determination of SlyB complex.

(FIG. 9A) Summary of the data processing strategies for the cryoEM 3D reconstruction of SlyB:BamA complexes (see FIG. 5 for raw particles and representative 2D classes). Steps performed using Relion⁵⁰ or Cryosparc⁴² are coloured green or red, resp. (FIGS. 9B and 9C) Fourier Shell Correlation curves for the final 3D reconstructions of SlyB oligomer (FIG. 9B) and SlyB:BamA (FIG. 9C) complexes. Average map resolutions mentioned throughout the manuscript are according to the FSC 0.143 criterion (shown as dotted line).

FIGS. 10A-10D. Structure properties of the 2™ glycine zipper domain.

(FIG. 10A) Helical wheel representation of the SlyB α1 and α2 helices that together form a conserved 2™ glycine zipper domain, and multiple sequence alignment of the corresponding regions of the six different 2™ Gly zipper domain containing proteins commonly identified in the E. coli genome (sequences show Uniprot ID and protein name, as well as MW of the full length proteins; numbering according to SlyB sequence). Hydrophobic and polar side chains are colored orange sand and white, resp. the conserved Gly or Ala residues in the GXXXG motifs forming the Gly zipper are colored magenta. Dotted lines connect opposing residues in the α1-α2 helical packing. (FIG. 10B) Ribbon representation of the 2™ Gly zipper domain as found in the SlyB structures reported in this study. Side chains are shown in stick representation, with N atoms colored blue, O atoms colored red and C atoms color coded according to (a). Shown as purple, yellow and magenta stick representation are the N-terminal lipid anchor, and a bound LPS and PL molecule in the SlyB protomers. LPS binds the loop connecting α1 and α2 by means of three H-bonds (red dotted lines), two between LPS fatty acids and the G79 and G80 backbone amine, and one between the D-glycosamine-4-phosphate and T82 side chain hydroxyl. In the transmembrane region α1 and α2 pack by the GXXXG knobs-in-hole pattern, with an additional stabilization of three H-bonds connecting the N- and C-terminal region of α1 and α2 resp. (i.e. N60 carbonyl—Q100 side chain amide and the N60 side chain amide with the Q103 side chain amide and the S104 hydroxyl). OM: outer membrane. (FIGS. 10C and 10D). Species wheel representation of the genomic distribution of the 2™ Gly zipper family (Rick_17 kDa_Anti; PF05433; https://pfam.xfam.org/family/Rick_17kDa_Anti#tabview=tab7). At least 12 discrete protein architectures containing the 2™ Gly zipper domain can be discerned, encompassing at least 4133 sequences originating from 1832 annotated species. Individual genomes hold 1 to 12 2™ Gly zipper containing sequences. The primary occurrence of 2™ Gly domain containing sequences is in Gram-negative phylum proteobacteria, with a smaller fraction in Ascomycete fungi. SP: signal peptide; 2™: 2™ Gly Zipper domain (green); OmpA: OmpA domain (blue; PFAM: PF00691; cell wall anchoring); Rick_17KDa: 17 kDa outer membrane surface antigen domain (fuchsia; PFAM: PF16998); CVNH: CyanoVirin-N Homology domain (yellow, PFAM: PF08881; glycan binding domain); LysM: LysM domain (magenta; PFAM: PF01476; peptidoglycan binding); b/g cryst: bacterial b/g crystallin domain (brown; PFAM: PF00030); RcnB: Nickel/cobalt transporter regulator domain (dark orange; PFAM: PF11776).

FIGS. 11A-11D. SlyB oligomerization, lipid nanodomain formation and OMP encapsulation.

(FIGS. 11A and 11B) Side and top view ribbon representation of two adjacent SlyB protomers in the SlyB₁₁ oligomer structure (see FIG. 3), with the 2™ domains, lipid anchor, LPS and PL colored as in FIG. 10; and the periplasmic domains colored blue to red from N- to C-terminus. Secondary structure elements are labeled α1-α2 and β1-β6 with A or B superscript for the left or right protomer, resp. Protomers interact by β-sheet augmentation in the periplasmic domain, i.e. strand β1 pairs with β4 of the adjacent SlyB protomer. In the transmembrane region, the protomers interact by non-specific hydrophobic contacts, so that SlyB does not form an obligate oligomer, and can also exist as a stable monomeric or non-circular, low MW oligomeric transmembrane protein. N76 and R84 are indicated as residues important for binding the luminal PL layer. (FIG. 11C) Side-by-side comparison of side and top view cryoEM 2D classes of the OmpC trimer, LMW SlyB:OmpC complexes and SlyB₁₈-OmpC complexes identified in a SlyB-OmpC double affinity purification. Top view classes of LMW SlyB:OmpC complexes did not show a well-resolved binding site for SlyB. Box size: 275 Å. (FIG. 11D) Schematic representation of SlyB:OMP nanodomains and side and top view cryoEM 2D classes of SlyB₁₄:BtuB and SlyB₁₂-TSX purified by double affinity purification. Box size: 275 Å. Top view classes show the presence of the BtuB or TSX β-barrels encapsulated by a SlyB oligomer of variable protomer number corresponding to the diameter of the enclosed OMP. β-barrels and SlyB oligomer are separated by a luminal PL bilayer, and SlyB oligomers are surrounded by a LPS ring. Side view classes show the inner core glycans protruding over the rim of the SlyB oligomer, as also seen for SlyB oligomers (FIG. 8) and SlyB:BamA complexes (FIG. 9). The N-terminal lipid anchor, PL, LPS and detergent micelle (M) are colored slate, magenta, yellow and light blue, resp.

FIGS. 12A-12C. Comparison of SlyB₁₃-BamA and BamABCDE holo complex.

(FIG. 12A) Side, periplasmic and extracellular views (from left to right) of the structure of the SlyB₁₃-BamA complex, with SlyB shown in ribbon representation (sand), the lipid anchor, LPS and PL shown in stick representation (slate, magenta and yellow, resp.). The embedded BamA molecule is shown as molecular surface (green). (FIG. 12B) Side and periplasmic view of the superimposition of the SlyB₁₃-BamA complex (shown in ribbon representation and colored as in (a)) and the BamABCDE holo complex (PDB: 5d0o Ref: 69), shown as molecular surface and colored BamA: sky blue; BamB: pink; BamC: red; BamD: blue and BamE: yellow. The overlay shows that for a SlyB₁₃ nanodomain, the Bam lipoproteins would go into steric clash with the SlyB periplasmic domains. (FIG. 12C) Superimposition of the BamA protomers as found in the SlyB₁₃-BamA (green) complex or the BamABCDE holo complex (sky blue). POTRA domains 1-5 are labelled P1-P5. In the SlyB₁₃:BamA complex P5 and P4 make an outward rotation (arrows) around the connector between P5 and the BamA β-barrel to avoid steric clash with the SlyB periplasmic domains. CryoEM maps for the SlyB₁₃-BamA complex showed only week density for the P3 domain and no density for P2 and P1, indicative of a large conformational flexibility in these domains.

FIG. 13. Multiple protein sequence alignment of bacterial SlyB proteins.

The amino acid sequences of SEQ ID NO:1-28 representing bacterial SlyB proteins were aligned herein to demonstrate the conserved motifs and regions of the SlyB family. Red-labeled white residues are identical in all represented SlyB proteins, boxed residue positions are highly conserved. The secondary structure elements corresponding to the different sequence motifs are indicated above the alignment, and the asterisk represents the N-terminal Cys residue of the SlyB mature domain (SEQ ID NOs: 57-84, resp.), which is modified by attachment of a lipid anchor during cleavage of the leader sequence.

FIGS. 14A-14D. SlyB is required for growth under OM stress conditions.

(FIG. 14A) Close-up of early exponential phase growth of E. coli BW25113 and derivative strains ΔslyB, ΔphoP and ΔslyB::slyB grown on LB and LB+1 mM EDTA. Calculated generation times for the respective strains and growth media. (FIG. 14B) OD₆₀₀ growth curves of E. coli BW25113 and derivative strains ΔslyB, ΔphoP and ΔslyB::slyB in N minimal medium with defined concentrations of Mg²⁺. Mean±SD (n=4 biological replicates). (FIG. 14C) OD₆₀₀ growth curves of E. coli BW25113 (WT) and BW25113 ΔslyB, transformed with ppmrA^{R815}. All cells grown in LB, with (I) or without (NI) 0.1% L-arabinose for pmrA^{R815} induction. Mean±SD (n=4 biological replicates). (FIG. 14D) OD₆₀₀ growth curves of E. coli BW25113 (WT), BW25113 ΔslyB, BW25113 ΔslyB attTn7::SlyB and BW25113 ΔslyB attTn7::SlyB^{TEVHIS} grown on LB and LB+EDTA (1 mM). Mean±SD (n=3 biological replicates).

FIGS. 15A-15C. The SlyB induction during stressed conditions.

(FIG. 15A) Quantitative analysis of total [SlyB](‘SlyB^{C}+SlyB^{M}’; i.e. denaturing SDS-PAGE) or oligomeric SlyB (SlyB^{C}; i.e. high MW in semi-native PAGE) by α-SlyB Western analysis of whole cell lysates at indicated time points post addition of buffer control (LB), 1 mM EDTA or 100 μg/ml Bactenecin (Bac2A). Plots show total [SlyB] or % SlyB^{C} relative to time 0. Mean±SD (n=3 biological replicates). (FIG. 15B) α-SlyB Western analysis of semi-native PAGE of BW25113 cells grown up to OD₆₀₀ of 0.1 on N minimal medium with indicated Mg²⁺ concentrations. (FIG. 15C) Western analysis of the semi-native SDS-PAGE of the SEC fractionated Ni-NTA pulldowns as in FIG. 2a, b and c. Western blot analysis use α-BamA, α-LptD, α-OmpA, α-OmpC or α-SlyB as primary immune serum; as indicated. MW: standards with molecular weight indicated in kDa.

FIGS. 16A-16C. Outer leaflet phospholipid triggers SlyB oligomerization.

(FIG. 16A) cryoEM Coulomb potential map of SlyB₁₂ shown in mesh representation carved (3 Å) around a SlyB monomer, viewed from the luminal side, and with map colored: orange (LPS), magenta (PL), light blue (SlyB) and blue (N-terminal lipid anchor, i.e. palmitic acid amide PLM and diacyl glycerol thioether, DAG). Selected residues are labeled: Cys18, Ile65, Val69 and Phe73. Inset: Coomassie-stained and α-LPS stained Western blot of SlyB^{C} complexes run on semi-native SDS-PAGE with (+) or without (−) heating (5 min 95° C.) of sample. LPS remains bound to SlyB^{C} under semi-native conditions. Map contoured at σ=2, with highlighted sections carved at 3.0 Å around the corresponding model coordinates. (FIG. 16B) Solvent accessible surface representation of the SlyB₁₂ model (colored sand and with 1 SlyB protomer in rainbow from blue (N-terminus) to red (C-terminus)), shown in side view (left) and cross-sectional side view (right). The bound LPS, PL and the N-terminal lipid anchor are shown in stick representation, and with cryoEM map shown as mesh, colored grey, blue and magenta, respectively. (FIG. 16C) Density gradient ultracentrifugation (2.02-1.44-0.77 M sucrose) of total membranes isolated from BW25113 ΔslyB with chromosomal complementation with slyB^{TEV_His} (left) or slyB^{PL} (right), a mutant (I65A, V69A, F73A) disrupting the outer leaflet PL binding site. Fractions (high to low density from left to right) are run on denaturing SDS-PAGE followed by Western analysis with α-SlyB or α-DsbD and α-OmpC as representative inner and outer membrane proteins.

FIG. 17. Proposed role of SlyB as outer membrane guard protein.

Proposed role of SlyB in the PhoPQ regulon as guardian of OM stability and OM protein homeostasis under OM stress conditions. LPS destabilizing conditions like loss of divalent metals or cationic antimicrobial peptide exposure result in PhoPQ activation. PhoPQ 1) induces pathways aimed at LPS production and remodeling to restore membrane stability and asymmetry, 2) induces the overproduction of OM lipoprotein SlyB, and 3) induces virulence pathways in enteric pathogens (species variable, pleiotropic function). Hypothetical model for SlyB activity as OM guard protein: OM destabilization results in LPS shedding and increased PL infiltration in the OM outer leaflet (1). Excessive outer leaflet PL and LPS demix into PL:PL bilayer islets, with increased fluidity and lowered tensile strength compared to LPS:PL bilayers, resulting in the loss of OM proteins from the OM (2). Increased [SlyB] and loss in lipid asymmetry act as biochemical switch to trigger SlyB oligomerization, resulting in the encapsulation of OM proteins residing in PL:PL islets into SlyB:OMP nanodomains (3). SlyB nanodomain formation guards OM stability during acute LPS destabilization, serving to prevent shedding of PL:PL islets and the associated fraction of the OM proteome. Under LPS stress, absence of SlyB results in localized breaches or punctures of the OM. OM: outer membrane; IM: inner membrane; PG: peptidoglycan.

FIGS. 18A-18G. SlyB acts as OM guard protein under LIPS stress conditions.

(FIG. 18A) α-SlyB Western analysis of semi-native PAGE of whole cell lysate of BW25113 (WT) and BW25113 ΔslyB::slyB^{PL} grown on LB with (+) or without (−) 1 mM EDTA. The SlyB^{PL} mutant is unable to form High MW complexes (SlyB^{C}). (FIG. 18B) Growth curves of BW25113, BW25113 ΔslyB and BW25113 ΔslyB::slyB^{PL} grown on LB with or without 1 mM EDTA. Mean±SD (n=4 biological replicates). (FIG. 18C) α-SlyB Western analysis of semi-native PAGE of whole cell lysates of BW25113 exposed to 1 mM EDTA, 100 μg/ml Bactenecin or 0.1 μg/ml LpxC inhibitor PF-04753299 show induction of SlyB^{C} formation. (FIG. 18D) Quantitative proteomics of BW25113 and BW25113 ΔslyB cell extracts, shown as log₂ fold change between cells grown on LB or LB+EDTA at 3 hours post inoculation (i.e. OD 0.4). Proteome fractions are labelled: OMP (red), OML (blue), periplasmic (orange), inner membrane (black), cytoplasmic (grey) or cell surface (magenta). N=3 biological triplicates. (FIGS. 18E and 18F) Quantitative Western analysis of cell- or OMV-associated BamA (i.e. in the cell pellet (e) or ultracentrifugation of culture supernatant) of BW25113 (blue) and BW25113 ΔslyB (orange) grown on LB and stressed for 30 min with (buffer; ‘LB’), 1 mM EDTA, 100 μg/ml Bactenecin or 0.1 μg/ml PF-04753299 (LpxC^{INH}). Cell associated [BamA] levels (e) are normalized to WT buffer control levels. N=3 biological replicates. OMV-associated BamA shown as absorption units. Null hypothesis was analyzed by unpaired t test where *: p<0.05, **: p<0.01 and ns: non-significant. (FIG. 18G) cryoTEM imaging of BW25113 and BW25113 ΔslyB whole cells, grown on LB or LB+EDTA (0.5 mM) or Bactenecin 2A (100 μg/ml). EDTA or Bac2A-stressed ΔslyB cells show localized breaches of the OM (black arrows), with outward bulging of the cytoplasmic membrane near larger OM breaches (white arrow). Scale bars 0.25 μm, or 0.1 μm (insets i, ii). OM: outer membrane, P: peptidoglycan layer, IM: inner membrane.

FIGS. 19A-19D. SlyB is essential under PhoP/Q inducing OM stress conditions.

(FIGS. 19A and 19C) OD₆₀₀ growth curves of E. coli BW25113 (WT) and derivative strains ΔslyB, ΔphoP and ΔslyB::slyB in LB or LB with indicated supplements, i.e. ‘EDTA’=1 mM EDTA (a, c); ‘Mg²⁺, Ca²⁺ or Mn²⁺’=10 mM of respective ion (c). Mean±SD (n=4 biological replicates). (FIG. 19B) Time lapse phase-contrast imaging of BW25113 and BW25113 ΔslyB or ΔphoP grown on LB agar with 5 mM EDTA. Black arrows indicate lysed cells. Images representative of >20 single colonies tracked, from at least 3 biological replicates. Scale bar=5 μm. (FIG. 19D) OD₆₀₀ growth curves of E. coli BW25113 (WT) and BW25113 ΔslyB, transformed with ppmrA^{R81S}. All cells grown in LB supplemented with 1 mM EDTA, and with (I) or without (NI) 0.1% L-arabinose for pmrA^{R81S} induction. Mean±SD (n=4 biological replicates).

FIGS. 20A and 20B. Structure determination of the SlyB:BtuB and SlyB:TSX complexes.

(FIGS. 20A and 20B) Summary of the data processing strategies for the cryoEM 3D reconstruction of SlyB:BtuB (a) and SlyB:TSX (b) complexes. Steps performed using Relion⁷⁴ or cryoSPARC⁴² are colored green or red, resp.

FIGS. 21A-21D. Immunization with SlyB OMD complexes invokes a strong, diverse and self-adjuvanting humoral immune response.

Antibody titers in sera obtained at day 63 from mice immunized with SlyB OMD complexes, with (FIGS. 21A and 21C) or without (FIGS. 21B and 21D) adjuvant (AddaVax) (indicated on top of each graph). Sera where tested for the presence of antibodies that bind to heath denatured (i.e. boiled, panel a, b) or folded (i.e. unboiled, panel c, d) E. coli OMPs BamA, LptDE, BtuB, or FhuA (indicated in legend on right of each graph). Data points show mean and sd epifluorescence signal ((AU) 800 nm) of sera obtained from two mice. The SlyB OMD complexes used for immunization represent SlyB nanodiscs that are detergent-extracted from the EDTA-stressed outer membrane of E. coli. SlyB OMDs are composed of SlyB and mixed cell envelope proteins.

FIGS. 22A-22H. Immunization with SlyB OMD complexes invokes a self-adjuvanting humoral immune response, comparable to adjuvanted immunization with isolated cargo antigens.

Antibody titers in sera obtained at day 63 from mice immunized with (see legends on the right of each graph): purified SlyB OMD or SlyB:BamA complexes, or purified OMPs (detergent solubilized BamA, LptDE, BtuB or FhuA). Immunizations using SlyB OMD or SlyB:BamA were injected in absence of adjuvant, detergent purified OMPs included the AddaVax adjuvant. Sera where tested for the presence of antibodies binding to heath denatured (i.e. boiled, FIGS. A, C, E, and G) or folded (i.e. unboiled, FIGS. B, D, F, and H) E. coli OMPs. Data points show mean and sd epifluorescence signal ((AU) 800 nm) of sera obtained from two mice.

FIGS. 23A and 23B. Immunization with SlyB OMD complexes invokes a strong, cell surface targeting humoral immune response.

Antibody titers in sera obtained at day 63 from mice immunized with (see legends on the right of each graph): purified SlyB OMD or SlyB:BamA complexes, or purified OMPs (detergent solubilized BamA, LptDE, BtuB or FhuA). Immunizations using SlyB OMD or SlyB:BamA were injected in absence of adjuvant, detergent purified OMPs included the AddaVax adjuvant. Sera where tested for the presence of antibodies that bind surface exposed epitopes in the cell envelope of whole cell E. coli strain MG1655 (FIG. 23A) and its deep rough mutant ΔrfaD (FIG. 23B). Data points show mean and sd epifluorescence signal ((AU)800 nm) of sera obtained from two mice.

### DETAILED DESCRIPTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein. The invention, both as to organization and method of operation, together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases ‘in one embodiment’ or ‘in an embodiment’ in various places throughout this specification are not necessarily all referring to the same embodiment but may.

### Definitions

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments, of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016), for definitions and terms of the art. Unless defined otherwise, all technical and scientific terms used herein have the same meaning as commonly understood by one of ordinary skill in the art (e.g. in molecular biology, biochemistry, structural biology, and/or computational biology).

“Nucleotide sequence”, “DNA sequence” or “nucleic acid molecule(s)” as used herein refers to a polymeric form of nucleotides of any length, either ribonucleotides or deoxyribonucleotides. This term refers only to the primary structure of the molecule. Thus, this term includes double- and single-stranded DNA, and RNA. It also includes known types of modifications, for example, methylation, “caps” substitution of one or more of the naturally occurring nucleotides with an analog. “Coding sequence” is a nucleotide sequence, which is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, recombinant nucleotide sequences or genomic DNA, while introns may be present as well under certain circumstances. “Gene” as used here includes both the promoter region of the gene as well as the coding sequence. It refers both to the genomic sequence (including possible introns) as well as to the cDNA derived from the spliced messenger, operably linked to a promoter sequence. “Promoter region of a gene” as used here refers to a functional DNA sequence unit that, when operably linked to a coding sequence and possibly placed in the appropriate inducing conditions, is sufficient to promote transcription of said coding sequence. “Operably linked” refers to a juxtaposition wherein the components so described are in a relationship permitting them to function in their intended manner. A promoter sequence “operably linked” to a coding sequence is ligated in such a way that expression of the coding sequence is achieved under conditions compatible with the promoter sequence.

With a “chimeric gene” or “chimeric construct” or “chimeric gene construct” is meant a recombinant nucleic acid sequence molecule in which a promoter or regulatory nucleic acid sequence is operatively linked to, or associated with, a nucleic acid sequence that codes for an mRNA, such that the promoter or regulatory nucleic acid sequence is able to regulate transcription or expression of the associated nucleic acid coding sequence. The regulatory nucleic acid sequence of the chimeric gene is not operatively linked to the associated nucleic acid sequence as found in nature, and may be heterologous to the encoding nucleic acid sequence molecule, meaning that its sequence is not present in nature in the same constellation as presented in the chimeric construct. More general, the term “heterologous” is defined herein as a sequence or molecule that is different in its origin. Thus “heterologous” refers also to two biological components that are not present together in nature. The components may be host cells, genes, or regulatory regions, such as promoters, or proteins. Although the heterologous components are not present together in nature, they can function together. For instance, a heterologous promoter, operably linked to a coding sequence, is heterologous but activates the expression of said coding sequence.

The terms “protein”, “polypeptide”, and “peptide” are interchangeably used further herein to refer to a polymer of amino acid residues and to variants and synthetic analogues of the same. A “peptide” may also be referred to as a partial amino acid sequence derived from its original protein, for instance after tryptic digestion. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers. This term also includes posttranslational modifications of the polypeptide, such as glycosylation, phosphorylation, ubiquitination, sumoylation, and acetylation, among others known in the art. Based on the amino acid sequence and the modifications, the atomic or molecular mass or weight of a polypeptide is expressed in (kilo)dalton ((k)Da). By “recombinant polypeptide” is meant a polypeptide made using recombinant techniques, i.e., through the expression of a recombinant or synthetic polynucleotide. When the chimeric polypeptide or biologically active portion thereof is recombinantly produced, it is also preferably isolated from or substantially free of culture medium, i.e., culture medium represents less than about 20%, more preferably less than about 10%, and most preferably less than about 5% of the volume of the protein preparation. By “isolated” or “purified” is meant material that is substantially or essentially free from components that normally accompany it in its native state. For example, an “isolated SlyB nanodisc”, “purified SlyB nanodisc composition”, “isolated SlyB OMDs”, or “isolated SlyB nanodisc particles” or “purified SlyB oligomer or proteins” refers to a (plurality of) discs, composition, complex or polypeptides which has been purified from the bacterial or host molecules which flank it in a naturally-occurring state, e.g., other membrane proteins or lipids as identified and disclosed herein which have been removed from the molecules present in the sample or mixture, or bacterial environment, such as a production host, that are adjacent but not part of the said SlyB nanodiscs, by using the detergents, or other agents, and/or purification means as disclosed herein, and as known in the art. An isolated protein or complex or oligomer or disc composition can be generated by amino acid chemical synthesis followed by further treatments or can be generated by recombinant production or by purification from a complex sample such as a bacterial culture. The term “molecular complex” or “complex” refers to a molecule associated with at least one other molecule, which may be a protein or a chemical entity. As used herein, the term “protein complex” or “complex” or “assembled protein(s)” refers to a group of two or more associated macromolecules, whereby at least one of the macromolecules is a protein. A protein complex, as used herein, typically refers to associations of macromolecules that can be formed under physiological conditions. Individual members of a protein complex are linked by non-covalent interactions. A protein complex can be a non-covalent interaction of only proteins, and is then referred to as a protein-protein complex; for instance, a non-covalent interaction of two proteins, of three proteins, of four proteins, etc. More specifically, a complex of the a membrane protein and another protein of interest, or a membrane protein and a nucleic acid molecule, optionally with other proteins or compounds bound to it.

“Homologue”, “Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived. The term “amino acid identity” as used herein refers to the extent that sequences are identical on an amino acid-by-amino acid basis over a window of comparison, preferably over the full length of the recited sequence. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, lie, Phe, Tyr, Trp, Lys, Arg, His, Asp, Glu, Asn, Gln, Cys and Met, also indicated in one-letter code herein) occurs in both sequences to yield the number of matched positions, dividing the number of matched positions by the total number of positions in the window of comparison (i.e., the window size), and multiplying the result by 100 to yield the percentage of sequence identity. A “substitution”, or “mutation”, or “variant” as used herein, results from the replacement of one or more amino acids or nucleotides by different amino acids or nucleotides, respectively as compared to an amino acid sequence or nucleotide sequence of a parental protein or a fragment thereof. It is understood that a protein or a fragment thereof may have conservative amino acid substitutions which have substantially no effect on the protein's activity. The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified”, “mutant” or “variant” refers to a gene or gene product that displays modifications in sequence, post-translational modifications and/or functional properties (i.e., altered characteristics) when compared to the wild-type gene or gene product. It is noted that naturally occurring mutants can be isolated; these are identified by the fact that they have altered characteristics when compared to the wild-type gene or gene product. A knock-out refers to a modified or mutant or deleted gene as to provide for non-functional gene product and/or function. It is noted that naturally occurring mutants or variants may be isolated; these are identified by the fact that they have altered characteristics when compared to the wild-type gene or gene product, and a different sequence as compared to the reference gene or protein.

“Binding” means any interaction, be it direct or indirect. A direct interaction implies a contact between the binding partners or interactors. An indirect interaction means any interaction whereby the interaction partners interact in a complex of more than two molecules. The interaction can be completely indirect, with the help of one or more bridging molecules, or partly indirect, where there is still a direct contact between the partners, which is stabilized by the additional interaction of one or more molecules. By the term “specifically binds,” as used herein is meant a binding domain which recognizes a specific target protein or specific target component or molecule, but does not substantially recognize or bind other molecules in a sample. Specific binding does not mean exclusive binding. However, specific binding does mean that proteins have a certain increased affinity or preference for one or a few of their binders.

The terms “subject”, “individual” or “patient”, used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, primates, avians, fish, reptiles, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes). However, it will be understood that the aforementioned terms do not imply that symptoms are present.

The term “treatment” or “treating” or “treat” can be used interchangeably and are defined by a therapeutic intervention that slows, interrupts, arrests, controls, stops, reduces, or reverts the progression or severity of a sign, symptom, disorder, condition, or disease, but does not necessarily involve a total elimination of all disease-related signs, symptoms, conditions, or disorders.

### DETAILED DESCRIPTION

The first aspect of the present invention relates to isolated or technically purified compositions, more specifically compositions comprising SlyB nanodiscs, which are isolated from a production system, more specifically from a production host, preferably from a bacterial production host its membrane structure. Said nanodiscs are obtained or obtainable by a method for producing said SlyB lipid discs, which involves to recombinantly express SlyB protein in a bacterial host, under conditions that trigger the increase in expression of SlyB, and consequently result in SlyB nanodomain formation in the bacterial membrane. So following the step of applying SlyB nanodomain-inducing conditions in a host cell, the SlyB nanodomains appear in the bacterial membrane as discoidal SlyB belts, enclosing a lipid bilayer nanodomain, which frequently captures outer membrane protein(s) in its lumen. To isolate the SlyB nanodiscs, as defined herein, those SlyB nanodomains are consequently extracted from the bacterial cells, preferably using detergent extraction, and the SlyB nanodiscs are specifically isolated from that extract using affinity and/or size separation techniques, to obtain the isolated SlyB nanodiscs, enclosing the lipid bilayer nanodomain as defined herein, and with a saccharolipid moieties surrounding the SlyB oligomeric belt, and typically containing one or more membrane proteins and/or macromolecules within the lipid nanodomain.

An isolated SlyB nanodisc or SlyB outer membrane nanodisc (OMD) as interchangeably used herein is defined herein as to comprise:
a discoidal SlyB oligomer, comprising two or more SlyB proteins,
a lipid bilayer nanodomain comprising one or more phospholipid layers,
a saccharolipid moiety surrounding the SlyB oligomer,
and typically one or more membrane proteins, and/or macromolecules (endogenous or heterologous to the production host)
   wherein said one or more membrane proteins or macromolecules are embedded in said lipid nanodomain and wherein the discoidal SlyB oligomer acts as membrane scaffolding protein enclosing the lipid bilayer nanodomain.

Nanodiscs as described herein thus refer to discoidal (or circular or ring-enclosed) lipid nanodomains in which a lipid bilayer is surrounded by a belt of membrane scaffolding molecules. In general, as known in the art, nanodisc belt protein are amphipathic molecules including proteins, peptides, and synthetic polymers, and formed by the use of certain apolipoprotein members as membrane scaffold protein (MSP), concerning artificially designed proteins comprising truncated forms of apolipoprotein (apo) A-I, wherein several helix elements are repeated or shuffled or engineered further, as to create diverse options for wrapping around the patch of a lipid bilayer to form a disc-like particle (Grinkova, et al. 2010; Protein Eng. Des. Sel. 23, 843-848). Nanodiscs as referred to in the art are synthesized by mixing together phospholipid/detergent micelles and MSP proteins, optionally with a membrane protein solubilized in detergent micelles for incorporation in the disc. During the process of nanodisc assembly, the amphipol polymers wrap around the hydrophobic patches of the membrane protein to form a stable complex in solution, followed by detergent removal. The advantage of using nanodiscs and thereby removing the detergent prevents damage of detergent compounds to the transmembrane domain's integrity and to its interfaces with the extracellular region (Scott & Aricescu, 2019; Curr. Opin. Struct. Biol. 54, 189-197).

As described in this invention, SlyB can be used as an alternative MSP to generate detergent solubilized nanodiscs, herein also referred to as “SlyB OMDs” or “SlyB nanodiscs”. SlyB nanodiscs enclose a lipid bilayer able to hold one or more membrane proteins, and consist of circular or discoidal SlyB oligomers that form by side-by-side positioning of SlyB protomers that are oriented perpendicular to the plane of the enclosed bilayer (as shown in FIG. 3 and FIG. 11). In SlyB nanodiscs, the SlyB proteins, which are not amphipathic molecules, thus act as belt proteins, and the assembly of SlyB nanodiscs is different from the ApoI-MSP-based nanodiscs in the sense that they are assembled within bacterial membranes through formation of SlyB lipid nanodomains, triggered upon increased expression of SlyB in the bacteria, which can consequently be isolated in a controlled and efficient manner using detergent-mediated extraction. So there is no detergent removal as in known nanodisc generation, but SlyB nanodiscs are detergent-solubilized particles, wherein macromolecules or proteins embedded in the lipid nanodomain are held in position and stabilized by the presence of the SlyB protein belt. So in a similar manner as for apolipoprotein-based nanoparticle systems, the SlyB nanodiscs are applied to provide a stable and more native-like environment to membrane proteins, be it in a stress-induced environment (while being in an artificial environment for the classical nanodiscs), though resembling the native membrane. Thus, the ‘SlyB nanodisc’ is defined herein as a nanoscale membrane system that assists in the solubilization, stabilization, and presentation of enclosed membrane proteins. Likewise to apolipoprotein or saposin-based nanodiscs (Frauenfeld et al. 2016; Nature Methods, 13(4):345-51), the SlyB nanodiscs may thus provide for a number of advantages compared to other systems for membrane protein solubilization and stabilization, in particular for ligand binding studies, analysis of conformational dynamics, and protein interaction studies (Wang, et al. 2015; Protein J. 34, 205-211), because of the more native-like environment and higher stability of proteins inside nanodiscs. Membrane protein/nanodisc complexes are advantageous to use in cryo-EM or crystallization studies because of their increased homogeneity, protection from aggregation, and conservation of conformational structure (Hagn, et al 2013; J. Am. Chem. Soc. 135, 1919-1925).

In a further embodiment of the invention, the isolated SlyB nanodiscs comprise at least a SlyB oligomer formed by at least two SlyB proteins, said SlyB proteins represented by the mature domain or mature protein form as shown in SEQ ID NOs: 57-84 derived of any of the amino acid sequences of SEQ ID NO:1-28, representing a selection of SlyB bacterial proteins identified herein as SlyB proteins by their homology in conserved regions essential for the typical SlyB fold and 3D structure (see also FIG. 13), or a further functional SlyB homologue with a mature domain of at least 80% amino acid identity of any one of those SEQ ID NO:1-28 sequences thereof, considered over the full length of the mature protein form of the SlyB SEQ ID NO:1-28 sequence, so over the full length of SEQ ID NOs: 57-84, resp., or a further SlyB mutant or variant sequence thereof. The SlyB mature domain is considered to be the sequence that remains after removal of the signal sequence (FIG. 13). It is understood that the full-length SlyB sequences represented in SEQ ID NO:1-28 correspond to SlyB pre-proteins, that are processed into SlyB ‘mature domains’ or ‘mature protein forms’, as used interchangeably herein, by proteolytic removal of the signal peptide, and attachment of a lipid anchor to the N-terminal Cys of their mature domain. The ‘signal peptide’, ‘signal sequence’ or also called ‘pre-sequence’ is a N-terminal sequence that directs the SlyB pre-protein to the general secretory pathway. In SlyB, the signal peptide comprises a lipoprotein signal peptide that ensures the pre-protein is transported by the Sec translocon and is cleaved by Signal Peptidase II (Lsp). Signal sequences can be predicted by means of bioinformatic tools as described Ref 70-71, and available through public servers such as the SignalP server (http://www.cbs.dtu.dk/services/SignalP/). So the mature SlyB proteins are defined herein as the SlyB proteins represented by the amino acid sequence after cleavage of the signal peptide. For the selected SlyB proteins of SEQ ID NO:1-28, as shown in FIG. 13, the mature SlyB protein forms are presented by the amino acid sequence as provided in SEQ ID NO:1-28 (and FIG. 13), minus the signal peptide at the N-terminus, as indicated in FIG. 13, resulting in the amino acid sequences of SEQ ID NOs: 57-84, resp. The bacterial SlyB homologues with at least 80% identity of the mature form are thus calculated as an amino acid sequence with at least 80% identity to any of the SEQ ID NO:1-28 over the full length of the mature protein form, lacking the signal peptide, or as an amino acid sequence with a mature domain as shown in SEQ ID NOs: 57-84 with a functional homology of at least 80% identity to any one of SEQ ID NOs: 57-84.

In a further embodiment said SlyB oligomer is formed by at least two SlyB proteins comprising amino acid sequences of SEQ ID NO:1-28, or a further functional SlyB homologue with a mature domain with at least 60%, 65%, 70%, 75%, 85%, 90%, 95%, 97%, or 99% amino acid identity of any one of those SEQ ID NO:1-28 sequences thereof, considered over the full length of the SlyB SEQ ID NO:1-28 sequence, or a further SlyB mutant or variant sequence thereof. In a further embodiment said SlyB oligomer is formed by at least two SlyB proteins comprising amino acid sequences of SEQ ID NO:57-84, or a further functional SlyB homologue with a mature domain with at least 60%, 65%, 70%, 75%, 85%, 90%, 95%, 97%, or 99% amino acid identity of any one of those SEQ ID NOs: 57-84 sequences thereof, considered over the full length of the SlyB SEQ ID NO: 57-84 sequence, or a further SlyB mutant or variant sequence thereof.

Another specific embodiment relates to the SlyB nanodiscs comprising the SlyB oligomer formed by at least 2 SlyB proteins comprising at least one SlyB mutant or variant sequence of any one of SEQ IDNO:1-28, or of SEQ ID NO: 57-84, resp., wherein a mutant contains at least one substitution, deletion or insertion of one or more amino acids, with an overall homology over the full length sequence of at least 60%, 70%, 80%, 90%, or 95% as compared to the original wild type sequence, and a variant refers to a conjugated or further adapted form of a functional SlyB protein, retaining its fold and function to form nanodiscs.

Another embodiment relates to the isolated SlyB nanodisc or membrane lipid discs or OMDs constituting a SlyB oligomer composed of at least 2 SlyB protomers, or preferably, at least 4, at least 5, 6, 7, 8, 9, 10, 11, 12, 13, 14, 15 protomers, and with a maximum of about 16, 17, 18, 19, 120, 21, 22, 23, 24, 25, 26, 27, 28, 29, or 30 SlyB protomers, wherein said SlyB protein sequences of the protomers may be identical or similar of variants or different SlyB proteins, such as a mixture of 2 or more different SlyB proteins, as defined herein, preferably with a mixture of SlyB proteins of any one of SEQ ID NOs: 54-87, or a homologue of at least 95% identity of any one thereof.

In specific embodiments, said isolated SlyB nanodiscs as defined herein have a size with a diameter of said nanodisc being below 40 nm, even preferably below 20 nm, more specifically between 9 and 15 nm. Indeed, the SlyB nanodomains, when present in a bacterial membrane are isolated as defined discs of which the size will depend on the number of SlyB proteins present in the belt or discoidal oligomer, and which typically is below 40 nm, thereby also differentiating the nanodiscs from OMVs, being vesicular structures that are larger and isolated from the bacterial membrane through a different isolation method.

Said SlyB nanodisc as described herein comprises at least any of the above SlyB oligomers, and further comprises a lipid nanodomain which contains at least one phospholipid layer, or bilayer, or multi-layered phospholipids, wherein the term “lipid nanodomain”, “lipid bilayer nanodomain”, also called “lipid rafts”, “membrane rafts”, or “membrane nanodomains”, as used herein, refers to compartmentalized parts of a (plasma) membrane which are enriched in a number of lipids such as for instance phospholipids, sphingolipids and sterol-rich lipid domains, and known to possibly recruit specific molecules or proteins, organized in lipid-protein assemblies by their interactions. Said lipid nanodomains are resistant to several detergents and hence typically isolated or extracted from the membrane or cells by detergent-mediated purification. In a specific embodiment, said compartmentalized part is present in the outer membrane of a Gram-negative bacterial cell, comprising one or more phospholipid layers. Said lipid nanodomain is thus encapsulated by ring-shaped SlyB transmembrane oligomers in the SlyB nanodisc, as present in the cell membrane, or as isolated from the membrane where it appears in SlyB nanodomains which were induced in SlyB inducing conditions. Compartmentalized OM nanodomains may vary in size and may or may not enclose one or more macromolecules, e.g., outer membrane proteins (OMPs) or foreign antigens. In the case of entrapment of proteins, lipid nanodomain size (diameter) and the number of SlyB monomers in an oligomer-ring complex may vary to encapsulate and stabilize embedded proteins, so the total mass of a SlyB nanodisc enclosing said OMPs will vary between 150 and 1000 kDa. In a specific embodiment, said isolated SlyB detergent solubilized particles have a size of 150 to 750 kDa, forming nanodisc structures of 9-12 nm width and approximately 7 nm height, comprising a circular SlyB oligomer encapsulating a lipid bilayer and embedded transmembrane protein. Thus, for any of such SlyB nanodiscs, with or without further macromolecule and/or membrane protein enclosed therein, the size will remain in the nanoscale with a hydrodynamic range of below 40 nm diameter, even below 30 nm, even below 25 nm, even below 20 nm, even below 18 nm, even below 15 nm, and with at least 5 nm, or at least 7 nm, or at least 9 nm, or at least 11 nm. For a conversion of protein mass to hydrodynamic range in nanometer see e.g. as defined in Erickson, (2009, Biological Procedures Online, 11 (1) p. 32).

It is envisaged herein that said SlyB nanodisc comprises at least any of the above SlyB oligomers, enclosing a lipid nanodomain as described herein, and further comprising one or more saccharolipid entities, preferably an outer saccharolipid moiety that is surrounding the SlyB proteins of the SlyB oligomer, preferably present at the periphery of the oligomer in a 1:1 ratio with the SlyB protomers. As used herein, the term “saccharolipid moiety” refers to chemical compounds containing fatty acids linked directly to a sugar backbone, forming structures that are compatible with membrane bilayers. In the saccharolipids, a monosaccharide substitutes for the glycerol backbone present in glycerolipids and glycero-phospholipids. Said saccharolipid moiety may thus be a lipid A component of the lipopolysaccharides in Gram-negative bacteria, or may for instance contain an lipooligosaccharide (LOS) or lipopolysaccharide (LPS) component, or even an engineered or modified amount or structure of a wild type LPS. In a specific embodiment, said saccharolipid component of the SlyB lipid disc is the most outside-facing layer of the SlyB lipid ring, belting the SlyB oligomers. The layer may comprise one more of the components such as detergent micelles, LPS ring and lipid A components of LPS. In said SlyB nanodisc structure, the SlyB oligomers are surrounded as a belt by said at least one outer saccharolipid moiety. The composition of the outer membrane may be subjected to modification to increase the suitability of the nanodiscs as a medicament, or as a composition administrable to a subject, or to be applied as an immunogenic composition. The SlyB nanodisc described herein comprises a SlyB oligomer, as described herein, enclosing a lipid nanodomain in its luminal side of the ring, and carrying one or more saccharolipid moieties surrounding said SlyB oligomeric ring or disc. The saccharolipid moiety as demonstrated and comprised in said isolated SlyB nanodiscs contributes to the self-adjuvating properties of the SlyB nanodiscs, when used as (part of) an immunogenic composition. It is clear to the skilled person in the art how to tweak and fine-tune the adjuvanting or immunogenic properties of the SlyB nanodiscs by for instance modifying the nature or abundance of the saccharolipid moiety in the SlyB nanodiscs. So in a specific embodiment, said saccharolipid moiety of the isolated SlyB nanodisc is a modified saccharolipid moiety, are a synthetic or unnatural saccharolipid moiety, and/or is present in the SlyB nanodisc at reduced or increased abundance or numbers in ratio to the SlyB protomers. Said ratio may be lower as 1:2, 1:4, 1:6, 1:8 or 1:10 saccharolipid moieties over SlyB protomers.

Said SlyB nanodisc composition comprises one or more isolated SlyB nanodiscs, as isolated from the host cell(s), and may contain further components beyond SlyB nanodiscs, such as macromolecules, which may be present as additional components of the composition, not linked, coupled or covalently fused to the SlyB proteins in the nanodisc; said macromolecules may be anchored at the outer nanodisc environment(s) in the composition, or the macromolecule(s) may be encapsulated or captured within the SlyB nanodisc(s) of the composition, preferably within the lipid bilayer nanodomain in the luminal side of the SlyB nanodisc(s). Also further individual particles or additional compounds and components present in said SlyB OMDs or nanodisc composition (as used interchangeably herein) are envisaged herein, such as further proteins, further chemicals, adjuvants, stabilizers, among others. The term “compound” describes any molecule, either naturally occurring or synthetic that is designed, identified, or generated, comprising organic and inorganic compounds. Compounds may also be more specifically “small molecules”, which refers to a low molecular weight (e.g., <900 Da or <500 Da) organic compound. The compounds also include chemicals, polynucleotides, lipids or hormone analogues that are characterized by low molecular weights. Other biopolymeric organic test compounds include small peptides or peptide-like molecules (peptidomimetics) comprising from about 2 to about 40 amino acids and larger polypeptides comprising from about 40 to about 500 amino acids, such as antibodies, antibody mimetics, antibody fragments or antibody conjugates.

In a further embodiment, said SlyB nanodisc composition comprises SlyB nanodiscs wherein at least one macromolecule is present within the lumen of said one or more nanodisc of the composition. The term “macromolecule” as used herein refers to a protein, a nucleic acid molecule, or a complex. When SlyB nanodiscs are produced in a host cell, the macromolecule is preferably directed to the cell membrane to be encapsulated within the forming SlyB nanodomains, from which the SlyB nanodisc/macromolecule complexes (or SlyB:OMP complexes, also defined herein as SlyB:OMDs) are consequently isolated. Said macromolecule may be an endogenously present macromolecule of the host cell producing the SlyB nanodisc, or may be an exogenously added macromolecule, the latter referring to a macromolecule foreign to or heterologous to, or recombinantly produced in the host cell. Said macromolecule may be expressed by any methods known to the person skilled in the art. Optionally, the macromolecule originates from the same bacterial species as the SlyB protein present in said nanodisc(s), alternatively, the macromolecule or a part thereof is heterologous to the SlyB protein of the nanodisc. When the heterologous protein is present as a fusion to the SlyB protein within the oligomer, the heterologous protein should have a molecular weight below 15 kDa, as not to disturb the formation of SlyB nanodomains for isolation of the SlyB nanodiscs containing the SlyB protein belt. Alternatively, the macromolecule is fused to the N-terminal part of a surface exposed membrane protein or lipoprotein of said host cell. In a specific embodiment, said macromolecule is a complex, which may be a complex of different types of macromolecules, or a complex of different proteins. In a specific embodiment envisaged herein, said macromolecule is a membrane protein, preferably a bacterial outer membrane protein or lipoprotein, specifically a beta-barrel-containing membrane protein, or a transmembrane protein, most specifically an outer membrane protein from a Gram-negative bacterium. The term “outer membrane protein” (OMP) as used herein refers to a polypeptide or protein integral to or attached to or expressed on the outer membrane of Gram-negative bacteria.

The protein may be an integral membrane protein, i.e. “embedded” within the membrane, optionally having portions exposed periplasmically and/or extracellularly. Alternatively, the protein may be attached to the extracellular surface of the outer membrane, either directly to the lipid bilayer or to an integral protein. Suitably, the outer membrane protein is between 600-1000 amino acids in length. Said macromolecule may also be a “carrier protein” to which another cargo or antigen is coupled or attached or conjugated, typically for the purpose of displaying or enhancing or facilitating detection of the coupled cargo or antigen.

In an alternative embodiment, the SlyB nanodisc composition as described herein further comprises an interactor protein of SlyB, which forms a complex with at least one of the SlyB protomers of the SlyB oligomeric nanodisc, by non-covalent or covalent binding, and preferably wherein said interactor protein comprises a YnfB protein as for example presented in amino acid sequence SEQ ID NO:29, or a YggE protein as for example presented in SEQ ID NO:30, or a bacterial functional homologue with at least 60%, 70%, 80%, 90%, 95%, or 97% identity of any one thereof, considered over the full length window of said protein sequence of SEQ ID NO:29 or SEQ ID NO: 30, or a mutant or variant thereof. Said SlyB-interactor complexes may be useful or more suitable in the formation of larger or better enclosed ‘cages’ for capturing macromolecules that are rather globular, soluble or charged in nature.

In certain embodiments, the macromolecule can be an immunogen or an antigen of a bacterial pathogen (infectious agent), a virus, and/or of a tumor or a further target. For example, the antigen can be from pathogens and infectious agents such as viruses, bacteria, fungi and protozoa. So in another embodiment, the macromolecule present in said SlyB nanodisc composition may be at least one macromolecule that has an identical or similar or variant structure or sequence as compared to a wild type macromolecule as present in a virus, a prokaryote, an eukaryote, a fungus a protozoan, a parasite, a pathogen, a mammal or human tumor or cancer cell, or a human or mammal neoplastic cell.

An alternative embodiment relates to said isolated SlyB nanodisc composition that is an immunogenic composition or a composition used as a vaccine. With an “immunogenic composition” or “vaccine” is meant herein a composition comprising at least one antigenic component which is capable of generating an immune response when administered to a subject. “Immune response” as used herein refers to a B-cell antibody and/or T cell response. An “antigen” or “immunogen” as used herein refers to a substance which stimulates an immune response in the body. More specifically, the antigen may be a nucleic acid such as a DNA or RNA molecule, or a polypeptide, such as a surface protein or a protein derived from the outer membrane protein of a Gram-negative strain. By “derived” in this sense is meant that the antigen is generated recombinantly or using said wild type antigenic structure as a starting point for modifying the antigen (e.g. by target mutation, truncation etc.) or intellectually (e.g. using the known sequence of said protein to design a synthesized polypeptide).

Indeed, the isolated SlyB nanodiscs, and composition comprising said discs, may be considered as an alternative bacterially-derived membranous nanoparticle composition to outer-membrane-vesicles (OMVs), capable of carrying and displaying cargo proteins, such as outer membrane proteins, to the immune cells of a subject. Although different from OMVs in that the isolated SlyB nanodiscs described herein are smaller in size, different in nature, since they at least do not comprise an inner periplasmic portion, and are detergent-solubilized particles, and in production/purification process, their immunogenic properties, which for OMVs has been shown to lead to protective mucosal and systemic bactericidal antibody responses in animal and human subjects, may be similar. OMVs were shown to be phagocytized and processed by antigen-presenting cells, which allows display of the OMV-contained antigens to CD4+ T cells, leading to the generation of antigen-specific B cell responses. In the OMV immunogenic response, lipo-polysaccharides function as potent activator of the immune cells.

The immune response raised by the isolated SlyB OMDs or nanodisc compositions may recognize SlyB as immunodominant component from its Gram-negative pathogenic host, or may recognize a further OMP present in the nanodisc composition, or even a further antigenic macromolecule or component present in said immunogenic composition. Alternatively, the immune response may be caused by displaying OMPs present on two or more strains, in which case said proteins need not be identical between the strains but must share similar epitopes such that each of the respective proteins is recognized by the immune response raised by the antigen. Of particular interest is the raising of an immune response which recognizes multiple, or all, strains of a given Gram-negative species, wherein said strains differ by at least one of serogroup, serotype, serosubtype or the precise amino acid sequence of the outer membrane protein from which the immunogenic composition is derived. Cross-protection may also extend beyond individual species. The immunogenic composition as envisaged herein comprises SlyB nanodiscs which may act as a vehicle, preferably a self-adjuvanting vehicle, further comprising endogenous OMPs from its production host, against which an immune response is desired, as to apply said immunogenic composition as a vaccine or as to raise antibodies specifically binding said OMPs, ultimately with said antibodies or immune response leading to a preventive or therapeutic effect against said pathogenic host. Alternatively, said immunogenic composition comprises isolated SlyB nanodiscs comprising a heterologously or recombinantly or exogenously added macromolecular antigenic determinant, as for instance exemplified herein for BamA and further bacterial antigens. Said immunogenic composition may be used for immunization or vaccination of a subject as to elicit an immune response against said exogenous macromolecule presented by the SlyB nanodiscs of said composition.

Another aspects relates to the isolated SlyB nanodisc, or the composition comprising the SlyB nanodisc, or the immunogenic composition described herein for use as a medicine. Alternatively, the isolated SlyB nanodisc, or the composition comprising the SlyB nanodisc, or the immunogenic composition described herein for use in prevention or treatment of a disease, such as a bacterial or viral infection.

A further embodiment relates to the isolated SlyB nanodisc, or the composition comprising the SlyB nanodisc, or the immunogenic composition described herein for use as a vaccine. So a further embodiment relates to a vaccine or a vaccine composition comprising the isolated SlyB nanodisc described herein, preferably for use in treatment or prevention of bacterial infection or disease. More specifically, an embodiment describes a vaccine comprising a first antigenic composition comprising the SlyB nanodisc described herein, or the immunogenic composition described herein, and optionally a second antigenic composition comprising a protein or nucleic acid of a pathogen or a human neoplastic cell or an animal neoplastic, tumor or cancer cell, wherein the pathogen is a virus, bacterium, fungus, protozoan, or parasite, wherein optionally said second antigenic composition is encapsulated in said first antigenic SlyB nanodisc or immunogenic composition. The SlyB nanodisc composition present in said vaccine may play a role as a carrier vehicle for the antigenic determinant of the vaccine, or alternatively the SlyB nanodisc may function as an adjuvant or as an antigen itself.

In a further specific embodiment, the immunogenic composition comprising the isolated SlyB nanodisc as described herein is used for eliciting a humoral immune response against the at least one endogenous or heterologous macromolecule embedded within the lipid domain of said SlyB nanodisc(s).

In a further embodiment, said vaccine, or immunogenic composition comprising the isolated SlyB nanodisc(s) described herein as a first antigen, has a second antigen wherein said second antigen is at least one of the OMPs or outer membrane lipoproteins of a bacterial target host, or recombinantly modified derivatives thereof, where recombinant modification may comprise the addition or modification of sequences that form immunogenic epitopes. Furthermore, said vaccine or immunogenic composition may further comprise another adjuvant moiety.

The vaccines or immunogenic compositions as described herein provide for pharmaceutical compositions, as known in the art, and can be administered orally, nasally, nasopharyngeally, parenterally, enterically, gastrically, topically, transdermally, subcutaneously, intramuscularly, in tablet, solid, powdered, liquid, aerosol form, locally or systemically, with or without added excipients or further adjuvants.

The vaccine or immunogenic compositions as described herein, comprising the SlyB nano disc composition isolated from a host cell, comprises at least one saccharolipid moiety, preferably a number of saccharolipid moieties, anchored to the/each SlyB protein(s) of said SlyB oligomer present in said SlyB nanodisc, wherein said saccharolipid moiety may be an LPS molecule, or a part thereof, or an LOS or a lipidA or part thereof, or a bio-engineered form of any one thereof. Pharmaceutical compositions for use to treat or administer to a subject preferably comprise SlyB nanodiscs with an altered or reduced LPS saccharolipid moiety, as to prevent toxicity, depending on the subject nature and dose.

The saccharolipid moiety of said SlyB nanodiscs preferably originates from the host cell or production host, being endogenous to the host, specifically a Gram-negative bacterial host. In a specific embodiment, the saccharolipid moiety of the SlyB nanodisc composition is obtained from a host that is bioengineered or modified in its LPS synthesis pathway, or from a cell culture that has been treated with LPS modifiers. LPS modification is possible in several genetic ways, as known by the skilled person, and is possible via chemical treatment. Moreover, LPS synthesis modification and/or LPS destabilization in the host membrane may be useful as to efficiently induce SlyB nanodomain formation in the host cell upon cultivation. In one embodiment, said Gram-negative bacterium is thus genetically modified to produce modified LPS with reduced endotoxicity as compared to Gram-negative bacterium without genetic modification, and preferably wherein the LPS at least retains partial adjuvant activity. The term “modified LPS molecule” as used herein refers to LPS that is modified for a reduction in toxicity as compared to its wild-type counterpart. Preferably said modified LPS has less than about 90, 80, 70, 60, 50, 40, 30, 20, 10, or 5% of the toxicity of its wild-type counterpart when administered to a subject. Any suitable assay that is known the person in the art can be used to measure the endotoxicity of wild-type and modified LPS forms and amounts. Preferably, modified LPS molecule or its Lipid A moiety that have reduced toxicity, retain at least part of its immunostimulatory, adjuvant activity. The saccharolipid moiety of the SlyB lipid disc may also be modified in the sense that fewer LPS are present on the SlyB lipid disc or oligomer, or that the SlyB lipid disc composition comprises SlyB lipid discs with LPS anchoring in a 1:1 ratio, mixed with SlyB lipid discs that have an altered saccharolipid anchor, with reduced or no endotoxicity. Overall, the LPS with reduced toxicity of the Gram-negative bacterium applicable in the SlyB lipid disc composition used as immunogenic composition, vaccine or pharmaceutical composition preferably has at least about 10, 20, 30, 40, 50, 60, 70, 80, 90 or 100% of the immunostimulatory activity of the corresponding wild-type LPS. The immunostimulatory activity may be determined by a method known by the skilled person, and is measured herein as a relative value over the wild type activity, for instance measured as the production of at least one cytokine or the expression of at least one costimulatory molecule upon co-cultivation of dendritic cells.

In one embodiment the SlyB nanodisc(s) are preferably obtained or obtainable from a Gram-negative bacterium with one or more genetic modifications in the LPS synthesis, which may lead to: a) a bacterium producing an LPS with reduced toxicity, wherein preferably the genetic modification reduces or eliminates expression of at least one of a IpxLI, IpxL2 and IpxK gene or a homologue thereof and/or increases the expression of at least one of a IpxE, IpxF and pagL genes; or b) preventing proteolytic release of cell surface-exposed lipoproteins from the bacterium, wherein preferably, the genetic modification reduces or eliminates expression of a nalP gene or homologue thereof.

In another embodiment, the isolated SlyB nanodiscs are preferably obtained or obtainable from a Gram-negative bacterial cell culture expressing SlyB in nanodomain-inducing conditions, wherein said cell culture has undergone a chemical treatment to modify the LPS content, and/or to destabilize the LPS composition or concentration in the membrane. For instance, reduced concentrations of divalent metal ions such as Mg²⁺ or Ca²⁺, preferably below 50 micromolar concentration, the addition of a metal chelator, or the addition of cationic antimicrobial peptides result in the destabilization of the outer membrane and its LPS composition. In another example, when chemical treatment with an inhibitor of LPS synthesis is used in cultivation of the SlyB nanodomain-forming bacteria, LPS concentrations in the outer leaflet of the outer membrane will be reduced, which in its turn affects the phospholipid content within said outer leaflet to be more abundant, resulting in formation of SlyB nanodomains, comprising SlyB OMDs which can be isolated thereof. In another example, alterations in the lipid composition of the outer membrane can be used to generate SlyB nanodiscs with an altered saccharolipid moiety content or modified LPS, usable as composition with reduced endotoxicity. One example of such an exogenous treatment may be obtained by using an inhibitor of LpxC, the first enzyme of the LPS synthesis pathway, to prevent LPS formation and thus deplete the bacterial cell culture from LPS, thereby destabilizing the outer membrane and inducing SlyB nanodomain and SlyB OMD formation, as demonstrated by SlyB promoter activity measurements (in cells where endogenous SlyB is present), and/or the presence of SlyB oligomers on native SDS-PAGE analyses of cell extracts, and optimally resulting in SlyB nanodisc compositions with altered or reduced saccharolipid moieties and reduced endotoxicity.

The Gram-negative bacterium where the SlyB nanodiscs may be produced, induced in, and isolated from preferably belongs to a genus selected from the group consisting of Neisseria, Brucella, Bordetella, Helicobacter, Salmonella, Vibrio, Shigella, Campylobacter, Haemophilus, Delftia, Diaphorobacter, Pseudomonas, Escherichia, Mannheimia, Moraxella, Coxiella, Chlamydia, Klebsiella, Legionella, Pasteurella, Yersinia, Acidovorax, Azotobacter, Aggregatibacter, Porphyromonas, Fusobacterium, Enterobacter, Francisella, Borellia, Bartonella, Rickettsia, Proteus and Acinetobacter, more preferably the bacterium belongs to a species selected from the group consisting of Neisseria meningitidis, Neisseria lactamica, Neisseria gonorrhoeae, Escherichia coli, Salmonella typhi, Salmonella typhimurium, Salmonella enterica, Helicobacter pylori, Vibrio cholerae, Shigella spp., Pseudomonas aeruginosa, Acinetobacter baumannii, Acinetobacter calcoaceticus, Borrelia burgdorferi, Brucella abortus, Francisella tullarensis, Porphyromonas gingivalis, Fusobacterium nucleatum, and Moraxella catarrhalis, specifically species infecting the respiratory tracts such as Brucella sp., Coxiella sp., Pseudomonas sp., Acinetobacter sp., Moraxella sp., Chlamydia psittaci, Chlamydia trachomatis, Klebsiella pneumoniae, Haemophilus influenzae, Haemophilus parasuis, Haemophilus somnus, Legionella pneumophila, Actinobacillus pleuropneumoniae, Bordetella pertussis, Bordetella parapertussis, Bordetella bronchoseptica, Mannheimia haemolytica, Pasteurella dagmatis and Pasteurella multocida.

Another aspect of the invention relates to a chimeric gene, which is composed of at least the following elements: a promoter to activate expression of SlyB, operably linked to a nucleic acid molecule comprising a coding sequence for a SlyB protein, preferably a sequence of a bacterial SlyB protein comprising SEQ ID NO:1-28 or a functional homologue with a mature domain as shown in SEQ ID NOs: 54-87, of at least 60%, 70%, 80%, 90%, or 95% identity of any one thereof, or a coding sequence for a SlyB protein functional variant of any one thereof or a mutant of anyone thereof. Said chimeric gene may further comprise operably linked DNA sequence encoding further proteins, which may be SlyB interactor proteins as defined herein, or further envisaged herein are protein fused or linked to SlyB. Alternatively, the chimeric gene may further comprise a nucleic acid molecule to encode or provide for the macromolecule additionally present in the production host cell or in the SlyB nanodisc composition as described herein. The promoter of said chimeric gene may be an endogenous promoter to the SlyB protein, in the sense that the promoter sequence originates from the same bacterial genus or species as the SlyB protein present in the chimeric gene. More specifically, the promoter may be a native SlyB promoter, or a SlyB promoter heterologous to the SlyB protein of said chimeric gene, but derived from a heterologous SlyB bacterial gene; or another bacterial promoter, preferably an inducible promoter, or alternatively said promoter may be heterologous promoter from a non-bacterial origin, or a synthetic promoter, preferably an inducible promoter, as known in the art. In a specific embodiment said chimeric gene does not comprise a nucleic acid sequence encoding a SlyB fusion protein wherein the fusion partner of SlyB is larger than 10 kDa.

In another aspect the invention relates to a host cell comprising the SlyB nanodiscs, the immunogenic composition, or the chimeric gene as described herein. The term “host cell” as used herein refers to any pathogenic, bacterial (Gram-negative or Gram-positive) or eukaryotic cell. A host cell according to the invention typically recombinantly expresses a SlyB protein, which may be a native SlyB protein of said host, in which case the endogenous SlyB encoded by the genome of said host will preferably be knocked-out in said host, and the native SlyB protein is expressed by introducing a chimeric gene comprising the native SlyB protein sequence for its recombinant expression. Alternatively, said host cell genome does not contain a SlyB-encoding gene and the SlyB nanodiscs are present in said host by recombinant expression or exogenous addition of a SlyB protein that is heterologous to the host. Said host cell expressing or comprising the SlyB oligomers forming nanodiscs may further contain exogenous macromolecules as part of the SlyB nanodisc composition described herein or as part of the immunogenic composition described herein. In an alternative embodiment said host cell comprising said recombinantly introduced SlyB nanodiscs may further be useful in isolation of SlyB nanodisc compositions, which preferably comprise additional components from said host cell, such as additional membrane compartments or parts, additional OMPs, or alternative protein components, or additional macromolecules. A host cell may be a pathogen and/or bacteria wherein one or more native OMPs are the macromolecules of/isolated with the SlyB nanodisc. A host cell may be a bioengineered cell which expresses/produces one or more foreign antigen and/or macromolecules.

In a preferred embodiment, the host cell comprising a recombinantly expressed SlyB protein, for forming SlyB nanodiscs, and preferably lacking endogenous SlyB, is a Gram-negative bacterium, from the genus and/or species as disclosed herein.

Another aspect of the invention relates to the use of the isolated SlyB nanodisc composition described herein. Said SlyB nanodisc composition may function as a chaperone or carrier or stabilizer for other molecules and therefore used for structural analysis of a protein of interest or a macromolecule of interest, encapsulated in said SlyB nanodiscs described herein. Specifically, the use of said nanodiscs is envisaged herein as an alternative to other nanoparticles or membranous nanodiscs known in the art, for providing an environment to a membrane protein or membrane component that is close to its native environment when aiming for structural analysis in vitro or even in vivo, by making use of the host cell comprising said SlyB nanodiscs as described herein. In a further embodiment said isolated SlyB nanodiscs or OMDs as described herein are provided for use in holding, positioning and/or stabilizing a protein enclosed in the lumen of the lipid bilayer nanodomain of said discs, and moreover, said enclosed protein preferably being a membrane protein, which may consequently be presented as an immunogen in vitro or ex vivo, and/or may be presented by the SlyB nanodiscs for insertion into a lipid membrane, in an in vitro or ex vivo system.

Further embodiments relate to the use of the isolated SlyB nanodisc composition as described herein in several biomedical applications such as liposome-based vaccines or lipid particle-based drug-delivery platforms; more specifically, specific SlyB nanodiscs may be applied to achieve cell-specific targeting and increase drug accumulation in targeted sites. Cargos could be covalently linked to the surface of SlyB nanodiscs or be encapsulated in the lumen of SlyB nanodiscs, providing for a use of the compositions described herein as vehicle.

Alternatively, said SlyB nanodisc composition or SlyB OMDs comprising bacterially derived membranous components may be useful as bacterial mimics and impair the adhesion and infection of bacteria to host cells, with the potential to administer SlyB nanodisc compositions as ‘anti-adhesion therapies’ for inhibiting the adhesion of bacteria to a subject's tissues and thus blocking bacterial infections.

A final aspect of the invention relates to a method to produce and preferably isolate the one or more SlyB nanodiscs or OMDs or SlyB nanodisc composition, as described herein, said method comprising the steps of:
a) inducing SlyB expression in a host cell, as to trigger SlyB nanodomain formation in the outer membrane of the host cell, and
b) extraction of the one or more SlyB nanodiscs from the SlyB nanodomains in the host cell membrane, preferably using a detergent, and
c) optionally, purifying the one or more SlyB nanodiscs from the extract using further purification techniques such as for instance size separation, and/or affinity chromatography.

In a specific embodiment, said SlyB expression is induced in a host cell by introducing a recombinant protein comprising a SlyB amino acid sequence, or a chimeric gene encoding a SlyB protein as described herein, preferably selected from any one of the sequences comprising SEQ ID NOs:1-28, or a sequence comprising the mature SlyB protein selected from any one of SEQ ID NOs: 54-87, or a homologue of any one thereof with at least 80% identity thereof.

A specific embodiment relates to the method for producing isolated SlyB nanodisc compositions wherein the step of isolation of the nanodisc particles is preferably performed by extraction of the nanodiscs through the addition of detergent to the cell culture, since the nanodiscs as described herein, are detergent-soluble and inherently resistant to disintegration by detergent use. “Detergent-mediated extraction” as used herein refers to a deliberate disruption of the bacterial membrane, by using detergent treatment (e.g., deoxycholate or sodium dodecyl sulfate, or any other detergent known in the art) to produce SlyB nanodiscs.

In a further specific embodiment, the induction of SlyB expression in a bacterial host, in step a), which triggers the formation of SlyB nanodomains or alternatively called “SlyB nanodomain-inducing conditions” or as used interchangeably herein “SlyB-inducing conditions” refers to any change in growth condition of a host cell culture that leads to an increase in SlyB protein and/or transcript level as compared to said host cells grown in non-stressed/unchanged/normal/native conditions. More particular, it is a surprising finding of the invention that native SlyB oligomers and nanodomain formation are induced to compartmentalize lipid nanodomains that stabilize the membrane proteome thereby by stress activation mechanisms. As exemplified herein, stress conditions trigger the induction of the stress regulon of the PhoP-PhoQ system and thereby seem also to activate SlyB expression in a manner that it is present in the outer membrane in an abundancy and conformation to form oligomeric structures to shield the outer membrane proteome, called SlyB nanodomains. It is also known in the art that the PhoP-PhoQ regulon or stress conditions inducing said signaling cascade is obtained by damage of the membrane or by affecting LPS stability or integrity in the outer membrane. So preferably, introduced SlyB is induced (i.e. “SlyB nanodomain-inducing conditions” are provided) in a Gram-negative bacterial host by a) induction of PhoPQ two-component system by applying stress, such as sensing low pH (i.e. pH below 5), divalent cation (e.g. Mg²⁺) shortage and cationic antimicrobial peptide (AMP) activity; b) by direct or indirect regulation of the genes encoding LPS modifying enzymes. In this case, the SlyB nanodomain-inducing conditions control the induction of SlyB expression by activation of a native or heterologous SlyB promoter that is introduced in the bacterial host, and thus is responsive to such stress-induced cascade. In specific embodiments, said SlyB nanodomain induction refers to induction of the expression in a host cell comprising an endogenous SlyB (as part of a Gram-negative bacterial host), or preferably induction of the expression in a host cell lacking an endogenous SlyB (as a SlyB knock-out or mutant strain), but having a recombinantly introduced SlyB protein, wherein said protein preferably comprises a sequence of any one of SEQ ID NO:1-28 or a bacterial homologue with a mature domain (as depicted in SEQ ID NOs: 54-87, resp.) of at least 80% identity thereof, or a mutant or variant thereof, capable of forming SlyB nanodiscs, and controlled by an inducible promoter. Ideally the host cell contains the chimeric gene as described herein, wherein SlyB expression is induced by activation of a SlyB promoter through any of the PhoPQ inducing conditions, or membrane and LPS destabilizing conditions. Alternatively, said chimeric gene comprises a heterologous promoter, which may be a bacterially-derived or synthetic promoter that is inducible by another artificial trigger known in the art, and allows to recombinantly overexpress SlyB in said host cell. In fact, the overexpression of SlyB, in limited period of cellular growth, is sufficient to induce SlyB nanodomain formation in the recombinant host.

So in a preferred embodiment, the SlyB nanodomain-inducing conditions comprise:
induction of the PhoP-PhoQ stress regulon of said host cell,
damaging the outer membrane and/or LPS destabilization of said host cell,
reducing the pH of the cell culture to pH 5 or lower,
addition of an cationic antimicrobial peptide to said cell culture,
depleting divalent cations, preferably by adding a metal chelator to said cell culture, or
addition of a SlyB agonist to said cell culture,
overexpression of an outer membrane protein,
or addition of an LPS synthesis inhibitor to said cell culture.

When recombinantly producing SlyB in said host cell by induction of a synthetic or bacterial inducible promoter system that leads to overexpression or ectopic expression of SlyB, the SlyB nanodomains will also form and be induced in a consistent and controllable manner, providing for a generic method to produce the SlyB nanodomains in any recombinant host cell that is suitable for the formation of the desired SlyB nanodisc compositions.

The method to produce and isolate SlyB nanodisc compositions thus further specifically involves the extraction of the SlyB nanodiscs, referred to herein as extraction by a detergent, and preferably followed by purification which may include conventional chromatographic purification, such as affinity purification, ion exchange, reverse-phase chromatography, hydrophobic interaction chromatography, size exclusion, and filtration, ultrafiltration, tangential flow filtration, centrifugation and gradient separations, among others. Preferably, since SlyB nanodiscs are isolated and distinguishable from the SlyB nanodomains in their smaller size, a size separation step is performed on the extract for isolating the SlyB nanodisc particles with a diameter below 40 nm, preferably below 20 nm, and with a molecular weight of maximally 1 Megadalton. With size separation techniques is referred to herein any methodology or chromatography known to the skilled person allowing to separate protein or macromolecules based on their size, such as for example gel filtration or size exclusion chromatography, or ultrafiltration, diafiltration, and tangential flow filtration, or hollow fiber filtration.

Said purified SlyB nanodisc composition may comprise additional endogenous or exogenous compounds present in the host cells, such as host cell proteins, membrane proteins or complexes, or endogenous or exogenously added antigens or macromolecules. In another embodiment said purified isolated SlyB nanodisc composition as described herein and obtained by the method of the present invention is used in a method further comprising the step of mixing or incubating a purified SlyB nanodisc composition with an exogenous macromolecule or mixture or sample, which results in encapsulation of at least one component of said exogenously added macromolecule or mixture or sample in said nanodiscs.

In a preferred embodiment, said method for producing and/or isolating the SlyB nanodisc composition relates to a method in which the host cell is a pathogen, preferably a Gram-negative bacterial pathogen, and more specifically is E. coli.

Another preferred embodiment relates to the method to produce the SlyB nanodisc composition wherein the host cell is not a wild type cell but is modified in that its endogenous SlyB is lacking or mutated, and/or in that its LPS synthesis is affected or modified.

In a further specific embodiment said method to produce and isolate the SlyB OMD particles provides for a host cell wherein the SlyB expression is induced through recombinant overexpression of a heterologous membrane protein, which in itself can trigger the increase in SlyB expression and SlyB nanodomain formation as to stabilize the membrane components of said host cells when the exogenous membrane protein is abundantly present in the host membrane. Said SlyB nanodiscs obtained from said method may be used as an immunogenic composition comprising the self-adjuvanting SlyB nanodiscs comprising the heterologous membrane protein enclosed in the lumen of the SlyB lipid bilayer nanodomain and presented by said nanodiscs as an antigenic determinant.

It is to be understood that although particular embodiments, specific configurations, compositions, as well as materials and/or molecules, have been discussed herein for methods, compositions and bacterial products according to the disclosure, various changes or modifications in form and detail may be made without departing from the scope of this invention. The following examples are provided to better illustrate particular embodiments, and they should not be considered limiting the application. The application is limited only by the claims.

### EXAMPLES

### Introduction

This study was set off by the serendipitous observation that the lipoprotein SlyB co-purified with the outer membrane assembly platform BamA when the latter was overexpressed (FIG. 5) in absence of its partner lipoproteins. Cryo electron microscopy (cryoEM) analyses showed an intricate complex formed by a circular SlyB oligomer enclosing a single copy of BamA. SlyB is one of three core components of the conserved PhoPQtwo-component system^{12,13} that monitors outer membrane integrity and stability by sensing low pH, Mg²⁺ shortage and cationic antimicrobial peptide (AMP) activity^{14,15}. AMP activity or shortage in divalent cations results in a destabilized and leaky OM by LPS shedding and increased PL infiltration in the outer leaflet, conditions that result in OM membrane blebbing and lysis if not countered⁷. To that effect, a primary outcome of PhoPQ activation is the upregulation of genes encoding LPS modifying enzymes that rigidify Lipid A by altering its acylation or by modifying its phosphates with aminoarabinose or phosphoethanolamine^{6,14,16}. The function of SlyB, however, remains largely obscure, despite its conserved position in the PhoPQ regulon and some reports hinting at a role in outer membrane integrity^{12,17,18}. Moreover, slyB can also be under control of the σE pathway¹⁹, which responds to dysfunction in OM biogenesis by sensing accumulation of LPS and unfolded OMPs in the periplasm²⁰. Upon activation, σE directs the increased transcription of genes implicated in LPS and OMP synthesis and stability such as the periplasmic chaperones, the Bam components and Lpx genes.

### Example 1. The PhoPQ Core Regulon Protein SlyB is Essential During LIPS Destabilization

We first investigated to what extend a ΔslyB knockout could phenocopy a ΔphoP mutant. Loss of PhoP function sensitizes E. coli to low [Mg²⁺], low pH and cationic antimicrobial peptides¹⁵ (FIG. 1a). Growth of the ΔslyB and ΔphoP mutants of E. coli strain BW25113 on LB agar or in LB broth was unaffected compared to that of their WT parent (FIG. 1b; FIG. 5). However, when grown on LB agar containing increasing concentrations of EDTA as a Mg²⁺ scavenger, BW25113 ΔslyB showed growth defects similar to those seen for BW25113 ΔphoP (FIG. 1a). Starting from 1 mM EDTA, BW25113 ΔslyB was attenuated in growth. In liquid cultures containing 5 mM EDTA, BW25113 ΔslyB had initial growth kinetics equivalent to its WT parent (38 and 37±2 minute generation time, resp.; FIG. 14a), but showed a loss in optical density after hours of growth (FIG. 1b). Time-lapse phase-contrast imaging of BW25113 ΔslyB grown in LB+EDTA showed cells to grow and divide for 3 to 4 generations before an abrupt arrest in growth and a sudden loss in contrast as a result of cell lysis (FIG. 1c). In contrast, WT cells continued expanding until reaching a stationary phase after approximately 12 hours (FIG. 1b,c). Although reaching a lower cell density than when grown in LB, WT cells grown in LB+EDTA had similar growth kinetics and showed no signs of lysis (FIG. 1c). The EDTA-stressed ΔphoP mutant showed a lower generation time (46±2 minutes) compared to WT or ΔslyB (FIG. 14a), but did not show the abrupt cell lysis seen for the latter (FIG. 19a,b). EDTA-induced lysis of BW25113 ΔslyB was sudden, indicating an acute loss of cell integrity (FIG. 1c). Chromosomal insertion of slyB under its native promotor at a distant locus (i.e. attTn7) restored EDTA resistance to WT levels, confirming the sensitization of BW25113 ΔslyB to the divalent cation scavenger results from a non-polar loss-of-function (FIG. 19a). Of notice, attempts to complement BW25113 ΔslyB by expression of slyB from plasmid resulted in growth attenuation and cell lysis, particularly when expressing the protein from an inducible promotor (FIG. 6). Similar to EDTA stressed BW25113 ΔslyB, the overexpression of SlyB resulted in sudden cell lysis after 6-7 generations post induction, indicating that non-physiological timing and/or levels of slyB transcription become toxic. The EDTA induced toxicity could be complemented by addition of an excess Mg²⁺, Mn²⁺ or Ca²⁺, indicating it resulted from a loss of divalent metal ions in general rather than a specific metal (FIG. 19c). Moreover, growth attenuation in BW25113 ΔslyB was specific to extracellular scavenging of divalent ions, since cells grown on minimal medium with limiting [Mg²⁺] showed growth characteristics equivalent to WT cells, unlike BW25113 ΔphoP, which showed reduced growth even at 2 mM Mg²⁺ (FIG. 14b). We reasoned that the conditional lethality of BW25113 ΔslyB related to an acute destabilization of LPS by loss of divalent counter ions, and that cells conditioned to low Mg²⁺ are desensitized to a SlyB requirement because of modification of LPS such as ethanolamine or aminoarabinose addition to the lipid A phosphates by activity of EptA and ArnT, respectively 6. In agreement with this hypothesis, a constitutively active pmrA mutant (i.e. R81S), which results in activation of eptA and arnT^{78,79}, suppressed the EDTA sensitivity of BW25113 ΔslyB to WT levels (FIG. 19d; FIG. 14c). We next assayed whether loss of SlyB also sensitizes E. coli to the other known PhoPQ activators, i.e. low pH and cationic peptides. The ΔslyB mutant showed an intermediate increase of pH sensitivity by 0.2 pH units compared to 0.4 pH units for ΔphoP cells (FIG. 1a). As an independent LPS destabilizing assault, we assessed the sensitivity of BW25113 ΔslyB to bactenecin A (Bac2A), a bovine leukocytic antimicrobial peptide that helps kill invasive pathogens by the disruption of cell envelope integrity²¹, mimicked that of the isogenic ΔphoP mutant (FIG. 6c). The BW25113 ΔslyB and ΔphoP mutants show full growth attenuation at 100 μg·mL⁻¹, conditions where the WT parent is unaffected in growth. Thus, we find slyB to be essential under an acute OM destabilization, conditions that can at least partially be alleviated by LPS modifying pathways induced by the PhoPQ sensory system.

As an example of environmentally induced LPS destabilization, we sought to investigate the role of SlyB in phagocyte survival of enteric pathogens like invasive E. coli and Salmonella enterica, where PhoPQ serves as a required response regulator for exposure to antimicrobial peptides and survival in the phagolysosome¹⁴, conditions associated with low pH and limiting Mg²⁺ concentrations²². Since we observed that the loss of slyB can in large part phenocopy ΔphoP, we generated ΔslyB mutants of uropathogenic E. coli strain UTI89, two poultry salmonellosis outbreak strains S. enterica sv. Enteritidis PT4 and S. enterica sv. Typhimurium χ3000 and the model murine typhoid strain S. enterica sv typhimurium LT2. Mouse monocyte-derived macrophages were infected with a 20-fold multiplicity of infection (MOI), and viable intracellular bacteria were monitored after 24 hours. Compared to the WT parents, mutant Salmonella strains showed a significant, ˜10-fold reduction in viable intracellular bacteria (FIG. 1d). Thus, SlyB function is required during host encounter of this invasive pathogen, and is at least in part responsible for the reduced intracellular survival of phoPQ mutants²³. UTI89 showed a lower but still significant reduction in intracellular survival (FIG. 1d), possibly due to a lower ability of phagocyte survival of this strain compared to Salmonella.

### Example 2. SlyB Encapsulates OMPs in Stress-Induced Lipid Nanodomains

Our prior finding that SlyB can bind and enclose apoBamA into a SlyB oligomer (FIG. 5) prompted us to systematically investigate the binding partners of SlyB. To do so, the coding sequence for a TEV-cleavable His-tagged SlyB (hereafter SlyB^{TEV_His}) under control of the native sly promotor was inserted at the Tn7 insertion site in the chromosome of E. coli BW25113 ΔslyB (FIG. 14d). Detergent solubilized cell extracts of bacteria grown in LB with or without 5 mM EDTA were subjected to two-step nickel-IMAC purification before loading on SDS-PAGE under denaturing (1% SDS, 5 min 95° C.) or semi-native (1% SDS, room temperature) conditions. Denaturing SDS-PAGE showed SlyB is present under non-stressed conditions, in agreement with previous studies that found a synthesis rate of 5.000-18.000 copies per generation in non-limiting media²⁴. EDTA stress resulted in an approximately 8-fold increase in SlyB concentration as well as in the co-elution of additional species ranging 25 to 100 kDa in apparent molecular weight (MW) (labeled * in FIG. 2a). Under semi-native conditions, a fraction of SlyB ran at the apparent MW of the monomer (SlyB^{M}), while a second fraction was found in the form of SDS-resistant high MW complexes (SlyB^{C}; FIG. 2a, FIG. 7). Similar sized SlyB^{C} complexes were also seen in SDS extracts of WT cells, within 5 minutes following EDTA- or Bac2A-induced LPS destabilization, increasing over time to occupy over 20% of cellular SlyB (FIG. 15a). SlyB^{C} formation was accompanied by increasing total cellular [SlyB]. Under non-stressed conditions, SlyB levels were stable and SlyB^{C} accounted for less than 5% of total [SlyB](FIG. 15a). Cells grown on N minimal medium with limiting [Mg²⁺] showed little SlyB^{C} formation (FIG. 15b), suggesting SlyB^{C} induction may be specific to acute LPS destabilization by EDTA or Bac2A.

When run over a size exclusion column, affinity-purified EDTA-induced SlyB^{C} eluted as a broad population of complexes ranging 150 to 1000 kDa apparent MW (FIG. 2b, FIG. 7). Semi-native PAGE of the SEC fractions revealed the presence of discrete SDS-resistant high MW complexes as well as a fraction of complexes that disintegrated into SlyB monomers in presence of SDS (MW: 13.8 kDa; FIG. 2c, FIG. 7). To determine the nature of the various SlyB complexes, SlyB pulldowns of cells grown in LB and LB+EDTA were submitted to peptide mass fingerprinting (FIG. 2d, e). Under LB conditions a select series of cell envelope proteins co-eluted with SlyB (defined as >10-fold change over control, and p<0.01; FIG. 2d), including OM beta-barrel proteins BtuB, PAL, TSX, OmpA, OmpC, MipA, FhuA, YfaZ and LptD; outer membrane lipoproteins YaeY and YiaD (both of unknown function); the inner membrane protein QmcA, and a single periplasmic protein of unknown function, YnfB. Under EDTA-stressed conditions, a much larger variety of cell envelope proteins co-eluted with SlyB, including 44 out of 57 identified outer membrane lipoproteins (OMLs; 77%), 22 out of 27 identified OMPs (81%), 20 out of 98 identified periplasmic proteins (20%), and 16 out of 124 identified inner membrane proteins (IMPs; 13%); compared to just 8 out of 906 cytoplasmic protein (0.9%; FIG. 2f). Thus, SlyB appeared to interact with integral OM proteins, particularly under EDTA-induced OM stress conditions, where up to 80% of the identified outer membrane proteome was found able to co-elute with SlyB. Western analysis of the size-separated SlyB pulldown with selected anti-OMP sera (OmpA, OmpC, BamA, LptD) confirmed their presence in high MW complexes (FIG. 7d, FIG. 15c). CryoEM imaging and reference-free single particle alignment showed the presence of discrete oligomeric complexes in the SlyB pulldowns (FIG. 2 g, h, FIG. 7e,f). A homogeneous group of classes (6 and 14% of the aligned particles in the LB and LB+EDTA pulldowns, resp.) corresponded to a 16-fold C symmetric complex of SlyB and the periplasmic protein YnfB (FIG. 2 g, h, FIG. 7e,f). In the LB pull, the majority of the remaining particles aligned into ring-shaped oligomeric complexes of variable diameter (˜84% of aligned particles; FIG. 7e), as well as in asymmetric low MW particles (SlyB_{LMW}; ˜10% of aligned particles; FIG. 2 g). All these complexes reveal the presence of a detergent belt surrounding the SlyB oligomers, indicative of their transmembrane nature. In the SlyB pulldown under OM-stressed conditions 2D classification revealed discrete SlyB:OMP complexes (˜86% of aligned particles, FIG. 2 g; FIG. 7f). These take the shape of (semi)circular SlyB oligomers surrounding an enclosed OMP, where the diameter and protomer number of the SlyB oligomers increases or decreases to match the size of the OMP. These SlyB:OMP classes included individually recognizable complexes such as SlyB:porin (i.e. OmpC or OmpF) and SlyB:TolC-like, as well as various OMPs that could not be unambiguously assigned based on the structural properties seen in the 2D classes.

To better understand the dynamics of the SlyB:OMP complexes we followed SlyB:BamA as an individual SlyB:OMP complex by co-overexpression of SlyB and BamA. Blue native PAGE of the SlyB pulldown showed the presence of a series oligomeric species with apparent MW ranging 500-600 kDa, and a second series of species ranging ˜180-220 kDa, corresponding to complexes containing, respectively, SlyB and BamA (i), and SlyB only (ii) (FIG. 2j,k). CryoEM and 2D alignment show SlyB:BamA complexes containing a single BamA surrounded by 13 to 14 SlyB protomers (FIG. 5e, f).

TABLE 1
Cryo-EM data collection, refinement and validation statistics.

| | | SlyB₁₁ | |
|---|---|---|---|
| | SlyB₁₀ | EMD-12950 | SlyB₁₂ |
| | EMD-12945 | PDB: 7OJG | EMD-12946 |
| Data collection and processing | | | |
| Magnification | 60 000 | 60 000 | 60 000 |
| Voltage (kV) | 300 | 300 | 300 |
| Electron exposure (e−/Å²) | 60 | 60 | 60 |
| Defocus range (μm) | 0.6-2.5 | 0.6-2.5 | 0.6-2.5 |
| Pixel size (Å) | 0.784 | 0.784 | 0.784 |
| Symmetry imposed | C10 | C11 | C12 |
| Initial particle images (no.) | 1 520 000 | 1 520 000 | 1 520 000 |
| Final particle images (no.) | 20 628 | 145 472 | 70 176 |
| Map resolution (Å) | 5.6 | 3.8 | 4.3 |
| FSC threshold | 0.143 | 0.143 | 0.143 |
| Map resolution range (Å) | 5.1-6.3 | 3.6-4.2 | 4.1-5.5 |
| Refinement | | | |
| Initial model used (PDB code) | | Ab initio | |
| Model resolution (Å) | | 3.8 | |
| FSC threshold | | 0.143 | |
| Model resolution range (Å) | | 3.6-4.2 | |
| Map sharpening B factor (Å²) | | −241.5 | |
| Model composition | | | |
| Non-hydrogen atoms | | 13,871 | |
| Protein residues | | 1760 | |
| Ligands (non-standard residues) | | 88 | |
| B factors (Å²) | | 35 | |
| Protein | | 26.31 | |
| Ligand | | 64.95 | |
| R.m.s. deviations | | | |
| Bond lengths (Å) | | 0.004 | |
| Bond angles (°) | | 1.109 | |
| Validation | | | |
| MolProbity score | | 1.89 | |
| Clashscore | | 7.29 | |
| Poor rotamers (%) | | 0.98 | |
| Ramachandran plot | | | |
| Favored (%) | | 91.91 | |
| Allowed (%) | | 8.09 | |
| Disallowed (%) | | 0 | |
| | | | SlyB₁₃-BamA |
| | SlyB₁₃ | SlyB₁₄-BamA | EMD-12949 |
| | EMD-12947 | EMD-12948 | PDB: 7OJF |
| Data collection and processing | | | |
| Magnification | 60 000 | 60 000 | 60 000 |
| Voltage (kV) | 300 | 300 | 300 |
| Electron exposure (e−/Å²) | 60 | 60 | 60 |
| Defocus range (μm) | 0.6-2.5 | 0.6-2.5 | 0.6-2.5 |
| Pixel size (Å) | 0.784 | 0.784 | 0.784 |
| Symmetry imposed | C13 | Local-Sym | Local-Sym |
| Initial particle images (no.) | 1 520 000 | 3 000 000 | 3 000 000 |
| Final particle images (no.) | 16 631 | 56 166 | 73 756 |
| Map resolution (Å) | 5.8 | 6.7 | 3.9 |
| FSC threshold | 0.143 | 0.143 | 0.143 |
| Map resolution range (Å) | 5.2-5.8 | 5.4-9.3 | 3.8-5.3 |
| Refinement | | | |
| Initial model used (PDB code) | | | 5D0O/SlyB₁₁ |
| Model resolution (Å) | | | 3.9 |
| FSC threshold | | | 0.143 |
| Model resolution range (Å) | | | 3.8-5.3 |
| Map sharpening B factor (Å²) | | | −86.2 |
| Model composition | | | |
| Non-hydrogen atoms | | | 20,293 |
| Protein residues | | | 2,292 |
| Ligands (non-standard residues) | | | 104 |
| B factors (Å²) | | | 56 |
| Protein | | | 53.04 |
| Ligand | | | 71.96 |
| R.m.s. deviations | | | |
| Bond lengths (Å) | | | 0.009 |
| Bond angles (°) | | | 1.192 |
| Validation | | | |
| MolProbity score | | | 2.21 |
| Clashscore | | | 14.45 |
| Poor rotamers (%) | | | 0 |
| Ramachandran plot | | | |
| Favored (%) | | | 90.13 |
| Allowed (%) | | | 9.87 |
| Disallowed (%) | | | 0 |

### Example 3. Structure of OMP-Free SlyB Oligomers and the SlyB-BamA Complex

To obtain a better insight in the SlyB complexes, we purified SlyB oligomers and SlyB:BamA and subjected these to cryoEM single particle analysis. 2D classification of SlyB oligomers showed particles of variable diameter and protomer number in agreement with the broad elution range of the protein on size exclusion chromatography (FIG. 2, FIG. 8a). Subsequent 3D classification of the SlyB oligomers converged to at least four well populated classes corresponding to C10, C11, C12 and C13 oligomers, representing respectively 8%, 58%, 28% and 6% of classified particles in the SlyB oligomer fraction used for cryoEM data collection (FIG. 8b). The more abundant C11 classes allowed a 3D reconstruction to 3.8 Å resolution, providing an Coulomb potential map that allowed unambiguous de novo building of the SlyB model (FIG. 3 a, b, c and FIG. 8, 16). The model contains the full residue range of the mature lipidated protein, comprising residues 18-155, as well as the N-terminal palmitic acid and partial density for the diacylglycerol moiety of the N-terminal lipid anchor (FIG. 3 a, c). The SlyB protomer folds into a 6-stranded split β-barrel like domain comprising residues 18-61 and 107-155, interrupted in the loop connecting strands β2 and β3 by an α-helical hairpin spanning residues 62 to 106 (FIG. 3a). α1 and α2 each consists of three consecutive GXXXG motifs, together representing a Glycine zipper 2™ domain (FIG. 10), a conserved putative two transmembrane α-helical region found in a variety of protein architectures in Gram-negative bacteria and ascomycete fungi (PFAM https://pfam.xfam.org/family/PF05433). The glycine zipper results in a tight right-handed packing of α1 and α2, which are otherwise comprised of hydrophobic residues (FIG. 10). Consistent with its prediction as a membrane-spanning region²⁵, the 2™ Gly zipper is flanked by a phospholipid molecule (PL) and the lipid A moiety of LPS on opposing sides of the α1-α2 hairpin, and the N-terminal palmitic acid is lining the groove between α1 and α2 (FIG. 3a, c; FIG. 10, FIG. 16). In the selected particles, 10 to 13 SlyB protomers align to form C-symmetry oligomers. Inter-protomer contacts are formed by a (3-sheet augmentation in the periplasmic domains, where β1 lines up with β4 of the adjacent protomer (FIG. 11). The 2™ domains line up into an α-helical barrel, but do so by unspecific hydrophobic contacts, suggesting the 2™ does not play a major driving force in SlyB oligomerization and can exist as individual transmembrane element, consistent with the observation of a fraction low molecular weight SlyB-OMP complexes comprising one to a few SlyB protomers only (FIG. 2; FIG. 11c). In the ring-shaped SlyB oligomers, the bound LPS molecules line the exterior of the particles, with the lipid A glycans and phosphates contacting the loop connecting α1 and α2 (FIG. 10, 11d; FIG. 16), and the inner core glycans forming a distinct ring protruding over the rim of the α-helical barrel. Remarkably, the bound PLs line the luminal side of the 2™ barrel and a cross-section of the SlyB oligomers shows continuous density with the expected width of a lipid bilayer (FIG. 3b). Thus, the Coulomb potential maps suggest SlyB oligomers enclose continuous lipid nanodomains of variable diameter (FIG. 3b; FIG. 11d).

In the 3D reconstruction of the SlyB:BamA complexes, two dominant particle classes correspond to 13- and 14-fold oligomeric SlyB nanodomains, with a single BamA copy enclosed in the lumen of the SlyB 2™ barrel (FIG. 5f; FIG. 9). The SlyB oligomer loses C-symmetry and instead partially follows the oval outline of the BamA beta-barrel. The SlyB₁₃:BamA allowed 3D reconstruction to 3.9 Å resolution, showing clear density for the BamA β-barrel (residues 424-808; in inward open conformation) as well as partial density for POTRA domains 5 and 4 (residues 347-420 and 269-346, resp.) (Table 1). BamA adopts the inward open conformation frequently seen in BamA apo structures as well as in the BamABCDE holo-complex²⁶. Remarkably, the SlyB:BamA complexes show no direct protein-protein contacts between BamA and the SlyB protomers. Instead, the proteins are separated by a single or multiple layers of lipid depending on the shape mismatch between the oval BamA barrel and the pseudo-C-symmetric SlyB 2™ barrel (FIG. 3f; FIG. 5f and FIG. 12a). Thus, SlyB:BamA complexes represent SlyB oligomers enclosing a lipid nanodomain with an embedded OMP rather than a direct contact with the cargo protein. This is further illustrated in SlyB:BtuB and SlyB:TSX complexes purified by tandem affinity purification (FIG. 11d). CryoEM 2D classes and 3D reconstructions of these complexes show a variable diameter SlyB nanodomains with an embedded OMP that is separated from the SlyB 2™ barrel by a variable width lipid bilayer. The Coulomb potential maps in these SlyB:OMP complexes again suggests a luminal PL bilayer rather than the canonical asymmetric PL-LPS bilayer comprising the OM FIG. 16 a & b).

### Example 4. SlyB Protects OM Stability and OMP Proteostasis Under LIPS Destabilization

Our structural and biochemical data show that SlyB assembles into oligomeric complexes that compartmentalize parts of the OM into lipid nanodomains. During OM stress, SlyB nanodomains contained individual OMPs. We reasoned that the isolation and encapsulation of OMPs may have a protective, stabilizing activity. To evaluate this hypothesis, we performed whole cell proteomics on WT and E. coli BW25113 ΔslyB cells under stressed and non-stressed conditions. In WT cells, EDTA-induced OM destabilization showed no significant change in OMPs or other cell envelope associated proteins other than a reduction in OmpC, and an increase in flagella and pilus subunits. The latter virulence factors are known to be upregulated during PhoPQ induction^{12, 13}. ΔslyB cells, however, showed a statistically significant decrease in OMPs, again with the exception of the virulence associated pilus assembly platform FimD (FIG. 18d). Cytoplasmic, inner membrane, periplasmic, or outer membrane lipoproteins did not show a similar unilateral decrease (FIG. 18d). Of note, the slyB mutant, but not WT, showed a significant increase in several cell envelope proteins involved in OM stability (Tol-Pal, Lpp), osmoregulation (OsmY, OsmE), and murein (FtsN, Slt) or phospholipid (GlpQ) remodeling, as well as a decrease in the OMP assembly associated lipoprotein BamC (FIG. 18d). Thus, in absence of slyB LPS destabilization results in reduced OMP levels and increased cell envelope remodeling & stabilization. We next followed cellular levels of BamA as a model OMP in WT or ΔslyB cells under different OM stress conditions. Under non-stressed conditions, BamA showed equivalent levels in both (FIG. 18e). However, treatment with EDTA, Bac2A or LPS depletion by PF-04753299 resulted in a small increase in WT cells, but a significant loss of BamA in the slyB mutant, within 30 minutes of exposure to the stressors (FIG. 18e). To test if SlyB nanodomain formation can have a stabilizing effect on an enclosed OMP, we purified SlyB:BamA nanodomains and apoBamA from EDTA stressed and non-stressed cells, respectively, and monitored the thermal stability of BamA (FIG. 4a). Thermal unfolding of apoBamA became evident from 62° C. onwards and reached half maximum (i.e. melting temperature or Tm) at 66.2° C. For the SlyB:BamA nanodomains, semi-native PAGE showed the folded BamA was part of SDS-stable HMW SlyB:BamA complexes. The SlyB-enclosed BamA showed an increased resistance to thermal unfolding, with a Tm of 72.0° C. BamA unfolding was concomitant with its release from the HMW complexes (FIG. 4a). To evaluate if SlyB:OMP nanodomain formation can stabilize BamA in its native OM environment, we performed a cellular thermal shift assay (CETSA) on cells treated with EDTA, where the melting temperature of BamA showed a ˜4° C. decrease in mutant versus WT cells (FIG. 4a), suggesting that under these stress conditions SlyB can bind at least a fraction of BamA in cellulo and may have a direct stabilizing activity on the OMP. Extracellular Mg²⁺ scavenging and AMPs result in an increased shedding of LPS and OMV formation. We therefore evaluated if the reduced OMP levels may also stem from excess vesiculation in ΔslyB versus WT cells. OMVs were spun down by ultracentrifugation and the levels of associated BamA were determined by Western analysis (FIG. 18 f). Loss of slyB resulted in a significant increase in [BamA] in OMVs isolated from cells grown on LB, LB+EDTA or LB+Bac2A. OMV formation could readily be observed by cryoEM inspection of WT cell treated with EDTA or Bac2A (FIG. 18 g). Strikingly, in addition to the presence of OMVs, the cell envelope of ΔslyB cells revealed the presence of distinct nanometer scale breaches in the OM (FIG. 18g). Periplasmic height and peptidoglycan—OM distance appeared largely intact in ΔslyB cells, indicating that OM organizing pathways like Tol-Pal, Lpp and OmpA^{73,74} are functionally intact. Instead, cells showed gaps in the OM, atop an otherwise continuous murein layer (FIG. 18g, inset i). We suspect that the lack of closure of the OM breaches is likely a result from the low fluidity and lateral diffusion of LPS-PL bilayers and an intact OM—murein association. Occasionally, the inner membrane could be seen to partially bulge through the OM gaps (FIG. 18g, inset ii). The presence of OM breaches was specific to ΔslyB cells and cells treated with EDTA or Bac2A. An accumulation of OM breaches may underlie the conditional lethality and abrupt lysis of OM stressed ΔslyB cells seen in phase contrast microscopy (FIG. 19b).

We have also monitored three model OMPs (BamA, FhuA and BtuB) by western analysis in whole cell extracts of E. coli BW25113 ΔslyB and its WT parent grown in LB at 30° C. with or without a 6 hour exposure to EDTA (FIG. 4b). In WT cells, exposure to EDTA resulted only in minor reductions of BamA, FhuA and BtuB levels. In sharp contrast, Mg²⁺ limitation in the slyB null mutant resulted in a 25 to 50% drop in the concentration of these OMPs, suggesting that SlyB nanodomain formation protects OMPs from denaturation and/or degradation under OM stress conditions. The level of protection slightly differed amongst the OMPs, suggesting that the reduction in OMP concentration is a function of their denaturation rather than a loss by means of OM shedding under EDTA stress conditions. To further evaluate that the SlyB protecting activity is indeed a direct function of reduced OMP denaturation and breakdown we monitored a temperature sensitive mutant of BamA (i.e. R661G/D740G²⁷), here referred to as BamA^{ts}). OM stress in cells lacking slyB resulted in a >70% reduction in BamA^{ts}, compared to only 25% in case of WT BamA. This suggest that the protective activity of SlyB:OMP complex formation under OM stress conditions results from a direct influence of SlyB on OMP denaturation and degradation.

### Example 5. Outer Leaflet PL Triggers SlyB Oligomerization and Nanodomain Formation

The structures of apo and OMP-enclosing SlyB nanodomains reveal the presence of a distinct PL binding site on the luminal side of the SlyB 2™ barrel (FIG. 3; FIG. 16). Remarkably, the orientation and location of these SlyB-bound PLs corresponds to the outer leaflet of the enclosed bilayer, suggesting SlyB oligomers enclose a PL-PL rather than a PL-LPS bilayer (FIG. 3 b; FIG. 16). A notion further supported by the sharp solvent boundary of the enclosed bilayer and lack of density corresponding to LPS inner core (FIG. 3b). To determine the significance of this outer leaflet PL, E. coli BW25113 ΔslyB was chromosomally complemented with slyB^{PL}, encoding a mutant SlyB with disrupted PL binding site (I65A, V69A and F73A; FIG. 18a; FIG. 16c). Although SlyB^{PL} got correctly sorted to the outer membrane, the mutant protein failed to induce nanodomain formation upon EDTA- or Bac2A induced LPS destabilization (FIG. 18a). Furthermore, whilst the SlyB^{PL} mutant showed WT growth levels under non-stressed conditions, it failed to rescue the conditional lethality of acute OM destabilization by EDTA or Bac2A (FIG. 18b). These OM assaults induce LPS shedding, resulting in a compromised OM asymmetry and increased outer leaflet PL levels 1. Together, these data suggest that excess outer leaflet PL acts as a biochemical trigger for SlyB oligomerization and nanodomain formation. To further test this hypothesis, we depleted cellular LPS levels by chemical inhibition of LpxC (PF-04753299; ⁷²) as an orthogonal condition resulting in a compromised OM lipid asymmetry. Exposure of E. coli BW25113 to PF-04753299 readily resulted in SlyB nanodomain formation (FIG. 18c).

### Example 6. Immunization with SlyB Nanodiscs

To test the ability of purified SlyB nanodiscs to elicit a humoral immune response against embedded outer membrane proteins, mice were immunized with SlyB nanodisc complexes (E. coli SlyB OMDs or SlyB:BamA) or isolated control antigens (BamA, LptDE, BtuB or FhuA) (See Methods). SlyB OMDs (see methods) are composed of SlyB and mixed cell envelope proteins, present as discoidal complexes of less than 20 nm, formed by a SlyB oligomer enclosing a lipid nanodomain that contains on average one OMP per individual SlyB nanodisc—see FIG. 2a-1 and FIG. 7.

Mice were immunized with five consecutive doses of the respective antigens, each dose formulated as 20 μg antigen diluted in 20 mM Tris pH 8, 150 mM NaCl, 0.03% DDM, and administered as intraperitoneal injection on day 0, 14, 28, 42 and 56, with a final bleed at day 63 to recover immune sera. SlyB OMDs and SlyB:BamA complexes were used as SlyB nanodisc antigens, each time with or without addition of AddaVax™, a squalene-based oil-in-water nano-emulsion to test the requirement of an adjuvant to elicit a strong humoral immune response. For immunization, the purified antigens (BamA, LptDE, BtuB or FhuA) were each supplemented with the AddaVax™ adjuvant. Two mice were immunized per antigen.

FIG. 21 shows the antibody titers (IgG1, IgG2a, IgG2b, and IgG3, IgM and IgA) in sera obtained at day 63 from mice immunized with SlyB OMD complexes (5×20 g in 20 mM Tris pH 8, 150 mM NaCl and 0.03% DDM) administered by intraperitoneal injection at day 0, 14, 28, 42, 56, with or without adjuvant (AddaVax). Sera where tested for the presence of antibodies that bind to heath denatured (i.e. boiled), or folded (i.e. unboiled) E. coli outer membrane proteins BamA, LptDE, BtuB, or FhuA. Antibody titers were determined by quantitative microfiltration dot blot analysis using anti-mouse secondary antibody (reactive to mouse IgG1, IgG2a, IgG2b, and IgG3, IgM and IgA) labelled for 800 nm fluorescence (see Methods). The SlyB OMD complexes used for immunization represent SlyB nanodiscs that are detergent-extracted from the EDTA-stressed outer membrane of E. coli. SlyB OMDs are thus composed of SlyB and mixed cell envelope proteins, present as discoidal complexes with diameter of less than 20 nm, formed by a SlyB oligomer enclosing a lipid nanodomain that contains on average one OMP per individual SlyB nanodisc—see FIG. 2a-1 and FIG. 7. As such, individual antigens represent less than 5% of the total antigen, with the exception of SlyB, which represents an ˜30% fraction of the antigen. Remarkably, the data show the presence of specific antibodies for each of the four antigens here tested, with titers as low as ˜1:50000 for BamA and LptDE, or ˜1:10000 for BtuB and FhuA in mice immunized with SlyB OMD complexes (FIG. 21). Moreover, robust humoral immune responses are seen also for the SlyB OMD immunizations lacking an added adjuvant (FIG. 21 b, d), demonstrating the self-adjuvanticity of the SlyB microdomains. Strikingly, the immune titers to both unfolded and folded OMPS are found to be larger in mice immunized with SlyB OMD complexes in absence of adjuvant (FIG. 21).

FIG. 22 shows antibody titers (IgG1, IgG2a, IgG2b, and IgG3, IgM and IgA) in sera obtained at day 63 from mice immunized with purified SlyB OMD or SlyB:BamA complexes, or detergent solubilized purified antigen OMPs BamA, LptDE, BtuB or FhuA (each time 5×20 μg in 20 mM Tris pH 8, 150 mM NaCl and 0.03% DDM) administered by intraperitoneal injection at day 0, 14, 28, 42, 56. Immunizations using SlyB OMD or SlyB:BamA were injected in absence of adjuvant, detergent purified OMPs included the AddaVax adjuvant. Sera where tested for the presence of antibodies binding to heath denatured (i.e. boiled, panel a, c, e, g) or folded (i.e. unboiled, panel b, d, f, h) E. coli OMPs. Same to above, in the SlyB OMD complexes used for immunization individual antigens represent less than 5% of the total antigen, with the exception of SlyB, which represents an ˜30% fraction of the antigen. Remarkably, the immune response in mice immunized with SlyB OMDs is found to be almost as strong, or even superseding that seen in mice immunization with the purified antigen itself, despite a >20-fold lower antigen concentration and the lack of adjuvant in the SlyB OMD samples, demonstrating the highly self-adjuvanting properties of the SlyB OMDs.

FIG. 23 shows the antibody titers (IgG1, IgG2a, IgG2b, and IgG3, IgM and IgA) in sera obtained at day 63 from mice immunized with purified SlyB OMD or SlyB:BamA complexes, or detergent solubilized OMPs (as above). Immunizations using SlyB OMD or SlyB:BamA were injected in absence of adjuvant, detergent purified OMPs included the AddaVax adjuvant. Sera where tested for the presence of antibodies that bind surface exposed epitopes in the cell envelope of whole cell E. coli strain MG1655 and its deep rough mutant ΔrfaD. Strikingly, the combined cell-surface targeting immune response in mice immunized with SlyB OMDs or SlyB:BamA complexes (titer as low as 1:50000) greatly exceeds that seen in mice immunized with individually detergent-solubilized OMPs (BamA, LptDE, BtuB and FhuA). Comparison of the immune response in the MG1655 strain and with its isogenic deep rough mutant ΔrfaD, shows the contribution of the LPS core glycan to the cell targeting immune response in SlyB OMD and Slyb:BamA immunized mice. A similar difference is not seen for mice immunized with the detergent-purified antigens, suggesting that the SlyB nanodiscs allow a more native-like presentation of the antigens compared to classic OMP—adjuvant immunizations.

### DISCUSSION AND CONCLUSION

The electrostatic stabilization of outer leaflet lipopolysaccharides by divalent metal ions plays a major role in ensuring the mechanical properties and essential barrier function of the OM in Gram-negative bacteria⁷⁷. LPS shedding induced by external stressors such as metal chelators or AMPs is accompanied by increased outer leaflet PL levels and has a strong destabilizing activity to the OM. Here we show that in response to a loss in OM lipid asymmetry the outer membrane lipoprotein SlyB oligomerizes into membrane enclosing nanodomains that encapsulate a large fraction of the OM proteome, a function that is essential during LPS destabilizing stresses. During such conditions, absence of a functional SlyB results in drastic declines of the OM proteome, nanometer scale breaches of the OM and abrupt lysis of cells. Strikingly, SlyB encapsulates lipid nanodomains that appear enriched in PL bilayers rather than the canonical PL-LPS bilayer (FIG. 3). Disruption of a luminal outer leaflet PL binding site on the SlyB monomers thwarts SlyB oligomerization and OMP encapsulation.

Spectroscopic evidence points to LPS and PL demixing and the segregation of excessive outer leaflet PL into PL bilayer islets or rafts^{1,29}. We speculate such PL islets to have increased fluidity and to lack the tensile strength of canonical PL-LPS bilayers, which may make the OM vulnerable to local rupturing. Our combined data support a function of SlyB as an OM guard protein, where excess outer leaflet PL act as trigger to oligomerization and nanodomain formation, an activity that is implicated in the maintenance of the OM proteome and the physical stability of the OM. We envision a model where increased PL imbalance during cation shortage or cationic peptide exposure results in a fraction of the OMP proteome residing in PL islets or ‘rafts’ rather than canonical PL-LPS bilayer. PL-induced SlyB oligomerizes compartmentalizes PL islets into SlyB nanodomains and encapsulates islet-associated OM proteins. The OM guard activity of SlyB may be dual, directly stabilizing encapsulated OMPs, and/or preventing the rupture and extrusion of PL islets (and associated OM proteins) from the OM. The stabilization of PL islets may stem from their compartmentalization, as well as from SlyB encapsulation of OM proteins that are in a stabilizing interaction with the cell wall.

Essential to SlyB nanodomain formation is its 2™ Gly zipper domain that can reside as monomeric transmembrane element as well as assemble into circular and elliptical oligomers of variable protomer number. At least five more 2™ Gly Zipper containing lipoproteins are seen in the E. coli genome (FIG. 16), all of unknown function. Of particular interest is YiaD, which includes a C-terminal OmpA like domain, and which was consistently found to co-purify with SlyB, suggesting it may have the ability to form mixed SlyB-YiaD oligomers. OmpA-like domains have cell wall binding properties in OM stabilizing proteins such as OmpA and Pal, suggesting YiaD may play a role in SlyB's OM stabilizing activity. However, it is unclear whether YiaD or any of the other 2™ Gly zipper proteins holds a cooperative, redundant or divergent function to SlyB.

In conclusion, we show that a small alphahelical TM hairpin protein can form variable size lipid nanodomains that have the capacity to encapsulate and stabilize embedded membrane proteins. SlyB and 2™ Gly zipper proteins are widespread across proteobacteria. In eukaryotes, the family is restricted to Ascomycetes, although it is conceivable that small, oligomerizing alphahelical proteins remain undetected that have a similar capacity to delineate lipid rafts and/or act as TM chaperones. We further show that the induced SlyB nanodomains can be extracted from the outer membrane in a format of detergent-soluble SlyB nanodisc particles, where SlyB is required as membrane scaffolding protein forming the belt which encapsulates a lipid bilayer and embedded proteins. Finally, it was demonstrated through mice immunizations that the application of SlyB OMDs—with envelope microbial proteins, or with co-expressed OMP antigens—provides for a self-adjuvanting vehicle for those OMPs, being capable of eliciting an immune response resulting in antibody titers at least comparable to the titers obtained using purified antigenic protein samples supplemented with an adjuvants, and superior in the fact that more native-like OMP-binding antibodies are obtained.

### Materials and Methods

Strains. Wildtype and derivative mutants of E. coli strains BW25113 and UTI89, LF82, and S. enterica sv. Typhimurium strains LT2 and χ3000 and sv. Enteritidis strain PT4 were grown in Lysogeny Broth (LB) at 37° C. while shaking, unless mentioned otherwise (Table 2). Where required, the culture medium was supplemented with the appropriate antibiotics at following concentrations: ampicillin 100 μg·mL⁻¹, chloramphenicol 25 μg·mL⁻¹ for plasmid-based resistance and 12.5 μg·mL⁻¹ for genomic resistance, kanamycin 50 μg·mL⁻¹, or spectinomycin 100 μg·mL⁻¹. E. coli and S. enterica knock-out strains were made using Datsenko and Wanner's single gene knock-out protocol³⁵ or through P1 (E. coli)³⁶ or P22 (S. enterica)³ phage transduction creating strains AJ1, AJ2, AJ3, AJ4, AJ5 and AJ6 (Table 2). In short, the pKD46 plasmid harboring encoding the phage λ Red recombinase was transformed into the target strain, followed by transformation with a linear DNA construct harboring a cm/R (chloramphenicol) or kanR (kanamycin) or speR (spectinomycin) resistance cassette and flanked by 50 bp homology regions of the target gene to knock out (Table 2). Complementation by genomic knock-in was performed using McKenzie and Craig's Tn7 directed gene insertion method³⁸, creating strains AJ1 attTn7::slyB, AJ1 attTn7::slyB^{TEV_His}, or AJ1 attTn7::slyB^{PL} containing the native sly promoter followed by, resp., slyB, a TEV-His-tagged version of slyB, or slyB^{PL} (i.e. encoding SlyB mutant I65A/V69A/F73A); and AJ5 attTn7::slyB and AJ5 attTn7::slyB^{PL} (Table 2). In short, target genes including native promotor were PCR-amplified using primers AJ102 and AJ103 (see Sequence listing) and cloned into the MCS of the pGRG25 vector (Table 2). Strains of interest were transformed with modified pGRG25 constructs (Table 2), and grown overnight under arabinose induction for the Tn7 directed recombination, before re-streaking on LB-agar incubated at 42° C. to cure cells from the heat-sensitive pGRG25. Expression plasmids were cloned using restriction-based cloning and/or an incomplete PCR based method. Details of the various expression constructs used in this study are reported in Table 2.

TABLE 2
Strains and plasmids.

| Strain | name | Description | Reference |
|---|---|---|---|
| E. coli BW25113 | | E. coli K-12 reference strain | (58) |
| E. coli BW25113 ΔslyB | AJ1 | slyB::cmlR by λ-red recombination with pKD3 PCR | This study |
| | | product using primers AJ93 and AJ94 | |
| E. coli BW25113 ΔslyB | AJ1 attTn7::slyB | AJ1 with slyB knockin at attTn7 using pGRG25_SlyB | This study |
| attTn7::slyB | | | |
| E. coli BW25113 ΔslyB | AJ1 | AJ1 with slyB^{TEV}^{_{—}}^{His} knockin at attTn7 using | This study |
| attTn7::slyB^{TEV}^{_{—}}^{His} | attTn7::slyB^{TEV}^{_{—}}^{His} | pGRG25_SlyB^{TEV}^{_{—}}^{His} | |
| E. coli BW25113 ΔslyB | AJ1 | AJ1 with slyB^{PL} knockin, a slyB mutant lacking PL | This study |
| attTn7::slyB^{PL} | attTn7::slyB^{PL} | binding, at attTn7 using pGRG25_SlyB^{PL} | |
| E. coli BW25113 ΔphoP | AJ2 | phoP::kanR by λ-red recombination with pKD4 PCR | This study |
| | | product using primers AJ97 and AJ98 | |
| S. enterica serovar | | Principal model strain for human salmonellosis and | (64) |
| Typhimurium LT2 | | mouse typhoid fever. | |
| S. enterica serovar | AJ3 | slyB::cmlR by λ-red recombination with pKD3 PCR | This study |
| Typhimurium LT2 ΔslyB | | product using primers AJ95 and AJ96 | |
| S. enterica serovar | AJ4 | phoP::cmlR by λ-red recombination with pKD3 PCR | This study |
| Typhimurium LT2 ΔphoP | | product using primers AJ161 and AJ162 | |
| E. coli LF82 | | Clinical isolate from patient with Crohn's disease (CD) | (80) |
| E. coli LF82 ΔslyB | AJ5 | slyB::speR by λ-red recombination with pBAD43 PCR | This study |
| | | product using primers AJ93 and AJ94 | |
| E. coli LF82 ΔslyB::slyB | AJ5 attTn7::slyB | AJ5 with slyB knockin at attTn7 using pGRG25_SlyB | This study |
| E. coli LF82 ΔslyB::slyB^{PL} | AJ5 | AJ5 with slyB^{PL} knockin at attTn7 site using | This study |
| | attTn7::slyB^{PL} | pGRG25_SlyB^{PL} | |
| E. coli LF82 ΔphoP | AJ6 | phoP::cmlR by λ-red recombination with pKD3 PCR | This study |
| | | product using primers AJ97 and AJ98 | |
| E. coli BL21-Al | | F− ompT hsdSB(rB − mB−) gal dcm araB::T7RNAP-tetA | Thermofisher |
| E. coli Stellar ™ Competent | | Chemical competent E. coli HST08 cells, used for | Takara Bio |
| Cells | | cloning | |
| J774A.1 | | Mouse BALB/c monocyte derived macrophage | ATCC ® |
| | | | TIB-67 ™ |
| Plasmid | name | Relevant information | Reference |
| pASK_IBA12_His | | pASK-IBA12 (IBA Lifesciences) modified to hold a | This study |
| | | SGGHHHHHHSGGGGG sequence between a N-terminal StrepII | |
| | | and thrombin recognition site | |
| pASK_IBA12_His_TEV_{—} | pBamA | Strep_His_TEV-tagged BamA expression vector: E. coli bamA | This study |
| BamA | | (residue 22-810) in pASK-IBA12_His | |
| pBAD43 | | pSC101 based expression vector with ara promotor and speR | (63) |
| | | resistance | |
| pBAD43_SlyB | | pBAD43 with slyB including native promotor | This study |
| pBAD43_pmrA | | pBAD43 with pmrA under Para control | This study |
| pBAD43_pmrA-R81S | | Encoding R81S mutant of PmrA, generated from pBAD43_pmrA | This study |
| | | using primers AJ159 and AJ160 | |
| pGRG25 | | Transposase vector for genomic knock-in directed to attTn7 with | (65) |
| | | oriT | |
| pGRG25_SlyB | | pGRG25 with slyB including native promotor, for genomic | This study |
| | | knockin at the attTn7 site | |
| pGRG25_SlyB^{TEV}^{_{—}}^{His} | | pGRG25 with slyB^{TEV}^{_{—}}^{His} under native promotor for genomic | This study |
| | | knockin at the attTn7 site | |
| pGRG25_SlyB^{PL} | | pGRG25 with slyB^{PL} under native promotor for genomic knockin | This study |
| | | at the attTn7 site | |
| pKD46 | | λ-red recombinase vector for genomic knock-outs with oriT | (60) |
| pKD3 | | Template vector for chloramphenicol cassette of genomic knock- | (60) |
| | | outs | |
| pKD4 | | Template vector for kanamycin cassette of genomic knock-outs | (60) |
| pASK_IBA12_His_TEV_{—} | pBtuB | Strep_His_TEV-tagged BtuB expression vector: E. coli btuB | This study |
| BtuB | | (residue 21-615) in pASK-IBA12_His | |
| pASK_IBA_SlyB_TEV_{—} | pSlyB | SlyB^{TEV}^{_{—}}^{His} expression vector: E. coli slyB (residue 1-155) with C- | This study |
| His | | terminal TEV and 6xHis in modified pASK-IBA12 (lacking OmpA | |
| | | signal peptide) | |
| pASK_IBA12_SlyB_TEV_{—} | pSlyB-BamA | Bicistronic expression of SlyB^{TEV}^{_{—}}^{His} and BamA; i.e. pSlyB + E. coli | This study |
| His_RBS_BamA | | bamA | |
| pASK_IBA12_SlyB_TEV_{—} | pSlyB-BtuB | Bicistronic expression vector for SlyB^{TEV}^{_{—}}^{His} and BtuB^{2xStrep}; i.e. | This study |
| His_RBS_BtuB_TwinStrep | | pSlyB + E. coli btuB with C-terminal 2xStrep | |
| pASK_IBA12_SlyB_TEV_{—} | pSlyB-OmpC | Bicistronic expression vector for SlyB^{TEV}^{_{—}}^{His} and OmpC^{2xStrep}; i.e. | This study |
| His_RBS_OmpC_TwinStrep | | pSlyB + E. coli ompC with C-terminal 2xStrep | |
| pASK_IBA12_SlyB_TEV_{—} | pSlyB-Tsx | Bicistronic expression vector for SlyB^{TEV}^{_{—}}^{His} and Tsx^{2xStrep} i.e. | This study |
| His_RBS_Tsx_TwinStrep | | pSlyB + E. coli tsx with C-terminal 2xStrep | |

Culture conditions. Growth curves were obtained by growing bacteria in 100 μL of N minimal medium (5 mM KCl, 7.5 mM (NH₄)₂SO₄, 0.5 mM K₂SO₄, 1 mM KH₂PO₄, and 0.1 mM Tris-HCl pH 7.4) supplemented with 0.1% Casamino Acids, 38 mM glycerol, and varying concentrations of MgCl₂ (0, 0.02, 0.2, 2 mM), or on LB medium, supplemented with OM stress stabilizing or destabilizing agents, as specified (MgCl₂, MnCl₂, CaCl₂, EDTA, Bactenecin 2A (Eurogentec), or PF-04753299 (Sigma-Aldrich)). Cells were gown in a 96-well plate with U shaped bottom wells (Falcon) in the Cytation One (Biotek) at 37° C. under double orbital shaking. For plasmid-based expression, media were supplemented with appropriate antibiotics, and cells were induced with 0.1% L-arabinose (Sigma-Aldrich) as mentioned. Bacterial growth was monitored by OD₆₀₀, measured every 30 min. On plate bacterial growth under OM stress conditions was monitored by streak plating 2 μL OD 0.5 inocula in star dish sectional petri dishes (phoenix biochemical) with LB-agar supplemented with varying EDTA concentration (0.5 mM, 1 mM, 2.5 mM, 5 mM) or pH (4.8, 4.6, 4.4, 4.2). Images were taken using the GelDoc (Bio-rad).

### Protein Production and Purification.

BamA production and purification (leading to the primary, serendipitous discovery of SlyB:BamA complex). The sequence encoding E. coli K12 BamA was PCR-cloned into pASK-IBA12_HIS vector with the N-terminal OmpA leader followed by StrepII tag, a 6×His tag and a TEV cleavage site (see sequence listing), forming pBamA. E. coli BL21AI was transformed with pBamA and grown in Terrific Broth (TB) at 37° C. At an OD₆₀₀ of 0.6-0.8 cells were induced by addition of 200 μg·L⁻¹ anhydrotetracycline and grown overnight at 30° C. with shaking. Cells were harvested by 15 min centrifugation at 5,000 g. Cells were resuspended at 1 g per 5 mL of lysis buffer, containing 50 mM Tris pH 8, 300 mM NaCl, 10 mM imidazole, 100 μg·mL⁻¹ lysozyme, 50 μg·mL⁻¹ DNAse I+5 mM Mg²⁺, 0.4 mM AEBSF, 1 μg·mL⁻¹ leupeptin, and 1.5% β-D-dodecyl maltoside (DDM). Cell lysis was performed at 4° C., over 2 hours while stirring. Lysate was cleared from insoluble debris by centrifugation (40,000 g, 4° C., 60 minutes). Recombinant BamA was purified using 2-step Ni-IMAC purification as follows: Strep-His-TEV-BamA was loaded on a pre-equilibrated HisTrap™ FF Ni-IMAC column (Cytiva), and contaminant E. coli proteins were removed by a 12 column volume (CV) wash with 50 mM Tris pH 8, 150 mM NaCl and 50 mM imidazole (buffer A). Native BAM lipoproteins (i.e. BamBCDE) that co-purified with Strep-HIS-TEV-BamA were eliminated with buffer A supplemented with 0.1% SDS before Strep-HIS-TEV-BamA was eluted with buffer A containing 300 mM Imidazole. The eluted fractions were pooled and incubated with TEV protease (TEV:protein, 1:20 w/w ratio) and dialysed against 50 mM Tris pH 8, 150 mM NaCl, and 10 mM imidazole overnight at 4° C. The proteins were then subjected to a second Ni-IMAC column to resorb Ni-binding contaminants. The untagged BamA was collected in the flowthrough and concentrated before loading on a Superdex 200 column (GE healthcare 16/600), pre-equilibrated with 20 mM Tris pH 8, 150 mM NaCl, 0.03% DDM. Peak fractions were analyzed with SDS-PAGE. Protein bands at 14 kDa and 180 kDa co-eluting with BamA were excised and analyzed by mass spectrometry fingerprint (FIG. 5).

Pulldown of SlyB complexes (from cells grown in LB and LB+EDTA for mass spectrometry). SlyB MS interactome experiments made use 2-step Ni-IMAC affinity purification of protein extracts of E. coli AJ1 attTN7::slyB and AJ1 attTN7::slyB^{TEV_His} (Table 2), containing a chromosomal knocking of the slyB gene with native promotor, and with (AJ1 attTN7:: slyB^{TEV_His}) or without (AJ1 attTN7::slyB; negative control) a C-terminal TEV-cleavable 6×His tag. Cells were grown in triplicate in two different conditions, LB and LB supplemented with 5 mM EDTA. Briefly, 50 mL culture was inoculated with 0.5 mL overnight starter culture. Cells were grown for 6 hours at 37° C. before harvesting by centrifugation at 5,000 g for 10 minutes. Individual cell pellets were resuspended in TBS buffer (50 mM tris pH 8.0, 300 mM NaCl) to a final OD₆₀₀ of 100, supplemented with 10 mM Imidazole, 50 μg·mL⁻¹ DNAse, 5 mM Mg²⁺, 0.4 mM AEBSF, and 1 μg mL⁻¹ leupeptin. The detergent DDM was added to the mixture to the final concentration of 1.5% for cell lysis and solubilization of total proteome, and incubated for 2 hours in cold room using a head-to-head rotator. Lysate clearing was done using a bench centrifuge at 14,000 g for 30 minutes at 4° C. SlyB affinity pulldowns were done using 2-step IMAC purification, (see method for BamA production and purification). However, pre-packed Ni-NTA columns were replaced by HisPur™ Ni-NTA Spin Columns, 0.2 mL (Thermo-Fisher). The final flowthroughs (SlyB pulldowns) were analyzed on a semi-native SDS PAGE and subjected to mass spectrometry.

Pulldown of SlyB complexes (from cells grown in LB and LB+EDTA for cryo-EM). See the section above for the pulldown procedure. To get enough materials for size exclusion chromatography and electron microscopy, 4 liters of culture was used in each condition, LB or LB supplemented with 5 mM EDTA. SlyB pulldowns after 2-step Ni-IMAC were applied onto a superose 6 increase column (GF healthcare 10/300), pre-equilibrated with 20 mM tris pH 8, 150 mM NaCl, 0.03% DDM. Fractions from each pulldown were checked by semi-native PAGE, screened by negative staining EM. Datasets were collected by cryo-EM.

Production and purification of SlyB apo complexes. E. coli BL21AI was transformed with pSlyB and grown in Terrific Broth (TB) at 37° C. The protein production and purification were performed as described for BamA production and purification (see above). After size exclusion chromatography, using a Superdex 200 16/600 column, peaked fractions containing SlyB were analysed using SDS-PAGE, semi-native PAGE and blue-native PAGE. Two fractions, the left slope of the first peak and the right slope of the second peak, were chosen for cryo-EM single particle analysis (FIG. 8a).

Production and purification of SlyB:OMP complexes (for cryoEM structure determination). To generate a bicistronic expression vector for the production of the SlyB:BamA complex, E. coli bamA including an RBS and its native signal peptide was cloned down-stream of the slyB gene in pSlyB, forming pSlyB-BamA (Table 2). The protein production and purifications were performed using 2-step Ni-IMAC purification as described above. The SlyB:BamA complexes were separated from SlyB oligomers and contaminants using a Superdex 200 16/600. Fractions containing the SlyB:BamA complexes were screened with negative staining electron microscopy and a fraction containing homogeneous SlyB:BamA particles was chosen for cryo-EM single particle analysis.

For the production and purification of SlyB:BtuB, SlyB:OmpC or SlyB:Tsx, a tandem affinity protocol was used. To do so, E. coli btuB, ompC or tsx including two consecutive C-terminal StrepII tags were cloned into pSlyB downstream of slyB^{T}EV_His and a RBS, to form pSlyB-BtuB, pSlyB-OmpC and pSlyB-Tsx, resp. (Table 2). Purification of the respective SlyB:OMP complexes was performed using batch 2-step His affinity purification as described above, and followed by a StrepII affinity step to separate the respective SlyB:OMPs from SlyB oligomers. After 2-step His affinity purification the flowthroughs containing the SlyB nanodomains were applied onto Strep-Tactin® XT Spin Columns (IBA-lifesciences, 2-4150-025) for the tandem affinity step pulling on the respective OMP partners. The columns were then washed with TBS buffer (50 mM Tris pH8, 150 mM NaCl, 0.03% DDM) and eluted with TBS buffer supplemented with 50 mM biotin. Purified complexes were analysed by SDS-PAGE and screened with negative staining EM before cryo-EM data collection.

Production and purification of control OMPs (BamA, LptDE, BtuB and FhuA). For the production and purification of BamA, BtuB, FhuA and LptDE a tandem affinity protocol was used using N-terminally His-TEV-tagged proteins expressed in E. coli BL21 using a modified pASK-Iba12 vector where the Strep-II tag was replaced by the coding sequence for a 6×His-TEV-tag, followed by the respective genes (i.e. E. coli K12 bamA, btuB, fhuA and lptD and IptE). In brief, total membrane proteins were extracted from the cells lysed in a buffer (using 10 mL per 1 g of cells) containing 50 mM Tris pH 8, 300 mM NaCl, 0.05 g/L lysozyme, 0.025 g DNAse l/L, 5 mM Mg²⁺ (added just before cell lysis), 1 mM DTT, protease inhibitors, and passed through a LM10 microfluidizer run at 12.000 Psi. Protein extraction was performed by addition of an equal volume of solubilization buffer containing 50 mM Tris pH 8, 300 mM NaCl, 10 mM Imidazole, 1 mM DTT, and protease inhibitors (optional). Supernatant containing the extracted membrane proteins is loaded on a Ni-IMAC column equilibrated in buffer A (50 mM Tris pH 8, 300 mM NaCl, 10 mM imidazole 10 mM, and 0.03% DDM, washed with 20 column volumes buffer A, before elution of His-TEV-tagged proteins by switch to buffer A supplemented with 300 mM imidazole. Eluted proteins are digested overnight at 4° C. with TEV protease, before a second passage of an equilibrated Ni-IMAC column. Pure target proteins are found in the flowthrough of the second Ni-IMAC column, concentrated and passaged over an S200 size exclusion column (GE Healthcare) equilibrated with 20 mM Tris pH 8, 150 mM NaCl, and 0.03% DDM.

### Structural Analysis Using Cryo-EM Single Particle Analysis.

Overall procedure. Protein samples were screened using negative staining EM. Briefly, samples were stained with 2% uranyl acetate and imaged using an in-house 120-kV JEM 1400 (JEOL) microscope equipped with a LaB₆ filament. For high-resolution cryo-EM analysis, sample grids were prepared by spotting 3 μl of sample (0.02-0.04 mg·mL⁻¹) on R2/1 holey grids (Quantifoil) coated with graphene oxide (Sigma Aldrich), manually blotted (3-4 s) and flash-frozen in liquid ethane using a CP3 plunger (Gatan). Sample quality was screened on the in-house JEOL JEM 1400 before collecting a dataset on a 300-kV CRYO ARM™ 300 (JEM-Z300FSC) Field Emission Cryo-Electron Microscope (JEOL) equipped with a K3 Summit direct electron detector (Gatan) at the VIB-VUB Bio-Electron Cryo-Microscopy center (BECM) in Brussels, Belgium. The detector was used in counting mode with a cumulative electron dose of around 60 electrons per Å² spread over 59-61 frames, at 60K magnification. Images were motion-corrected with MotionCor1.3³⁹ and defocus values were determined using CtfFind4⁴⁰. Dose-weighting scheme was applied. Particles were picked with crYOLO⁴¹ using a pre-trained model from a subset of manually picked particles with threshold 0.1. The box files were imported to RELION for particles extraction. Particle stack file was then exported to cryoSPARC⁴² for multiple rounds of 2D classification for junk particle filtration. Good particles were imported back to RELION for 3D alignment and reconstruction. Rotational and local symmetry were applied for the apo SlyB complexes and SlyB:BamA complexes, respectively. Map conversion and sharpening was done using PHENIX auto_sharpen⁴³, and SlyB models were built de novo using COOT⁴⁴. Refinement was performed using PHENIX real-space refinement⁴⁵ in combination with manual rebuilding using COOT. Geometry restraints for non-standard ligands (i.e. N-terminal diacyl glycerol and S-palmitoyl-L-Cys thioester; LPS) were generated by extraction of idealized coordinates of component residues from the PDB or PubChem (GOL: glycerol; PLM: palmitic acid; DAO: lauric acid; MYR: myristic acid; FTT: 3-hydroxy-tetradecanoic acid; PA1: 2-amino-2-deoxy-alpha-D-glucopyranose; GCS: 2-amino-2-deoxy-beta-D-glucopyranose; KDO: 3-deoxy-D-manno-octulonsonic acid; GMH: L-glycero-alpha-D-manno-heptopyranose; GLC: glucose; A4N: 4-amino-4-deoxy-L-arabinopyranose; DPO: diphosphate; P04: phosphate), manual building of the ligands and covalent LINK descriptions, followed by semi-empirical quantum mechanical geometry optimisation (AM1) and restraint generation using PHENIX eLBOW⁴⁶. Three-dimensional reconstructions were displayed, and figures were prepared in USCF Chimera⁴³ and PyMOL (https://pymol.org/2/).

SlyB oligomer cryo-EM single particle analysis. 6,352 movies composed of 61 frames at a defocus range between 0.6 and 2.5 μm were collected at 0.784 Å·pixel⁻¹. Motion correction, ctf estimation and particle picking were performed as described above, resulting in 1.52 million particles, binned 4. After 3 rounds of 2D classification, in cryoSPARC, 756,227 particles retained and converted to a star file using csparc2star.py for 3D classification in RELION. To ease the 3D classification, the first round, C12 symmetry was applied to all particles (FIG. 8). The best defined class was then was then selected and re-extracted without binning. Further 3D classification and 3D refinement using C12 symmetry were performed, leading to a Coulomb potential map of 4.3 Å resolution consisting of 70,176 particles. The classes from the first 3D classification with obviously smaller sizes were combined and subjected to another 3D classification with C11 symmetry. Two classes that agree with C11 symmetry were selected and a new particle subset was re-extracted without binning. Further 3D classification and refinement, resulting in a 3D map of 3.8 Å resolution with 145,472 particles. Additionally, the class with the smallest defined SlyB particle was selected and re-extracted for C10 symmetry 3D classification and refinement, resulting in a 3D map of 5.6 Å resolution with 20,628 particles. A final 3D class corresponding to bigger sized particles was selected and re-extracted for C13 symmetrized SlyB₁₃ reconstruction, which led to a 3D map at 5.8 Å resolution with 16,631 particles (FIG. 9). See Table 1 for data and model statistics. 3D classification and refinement of this dataset were also performed using cryoSPARC⁴², resulting in equivalent maps (not shown).

Processing of SlyB-BamA complexes. 7,352 movies composed of 61 frames at a defocus range between 0.6 and 2.5 μm were collected at 0.784 Å·pixel⁻¹. After motion correction and ctf estimation, 7,014 good images were retained. CrYOLO picking using a pre-trained model led to 3.0 million particles. After 3 rounds of 2D classification in cryoSPARC, 792,005 particles were retained and converted to RELION star file for 3D classification and refinement. Multiple rounds of 3D classification and refinement resulted in 2 main populations of SlyB:BamA complexes: SlyB₁₃:BamA and SlyB₁₄:BamA. Using local symmetry for SlyB monomers pushed the resolution of SlyB₁₃:BamA complex to 3.9 Å resolution with 73,756 particles while SlyB₁₄:BamA complex was reconstructed at 6.7 Å resolution with 56,166 particles (FIG. 9). To prepare maps and masks for local symmetry, 3D reconstructions were displayed and segmented using the SEGGER module implemented in UCSF Chimera⁴⁷. The SlyB13:BamA model was build using manual rigid body docking of SlyB monomers derived from the SlyB₁₁ model (see above) and BamA (derived from PDB ID: 5D0O) into the Coulomb potential maps, followed by manual rebuilding using COOT⁴⁴ and PHENIX real-space refinement including NCS averaging and secondary structure restraints⁴⁵. See Table 1 for data and model statistics.

Processing of SlyB-BtuB complex. 16,515 movies composed of 61 frames at a defocus range between 0.8 and 2.6 μm were collected at 0.76 Å·pixel⁻¹. The movies were motion corrected and ctf estimated. 3,515,932 particles were picked using CrYOLO⁴¹ with a general model. Particles were extracted, binned 4×, using RELION⁷⁵ and exported to cryoSPARC⁴² for three rounds of 2D classification, resulting in 576,212 well defined single particles corresponding to the SlyB-BtuB complex. These selected particles were re-extracted without binning and imported into cryoSPARC⁴² for 3D classification and refinement. Non-uniform refinement led to a reconstructed map of SlyB₁₄:BtuB at 3.86 Å resolution, (FIG. 20; FIG. 11d). SlyB₁₄:BtuB model was built by rigid body docking of SlyB monomers derived from the SlyB₁₁ model and BtuB (PDB ID: 1NQE) into the SlyB₁₄:BtuB reconstructed map, followed by manual rebuilding using COOT 44 and PHENIX real-space refinement including NCS averaging and secondary structure restraints 45. See Table 1 for data and model statistics.

Processing of SlyB-Tsx complexes. 15,393 movies composed of 61 frames at a defocus range between 0.8 and 2.6 μm were collected at 0.76 Å·pixel⁻¹. The movies were motion corrected and ctf estimated. 4,796,767 particles were picked using CrYOLO⁴¹ with a general model. Particles were extracted, binned 4×, using RELION 75 and exported to cryoSPARC⁴² for three rounds of 2D classification, resulting 1,219,574 well defined single particles corresponding to the SlyB-Tsx complex. These selected particles were re-extracted without binning and imported into cryoSPARC⁴² for 3D classification and refinement. Non-uniform refinement led to reconstructed maps of SlyB13:Tsx and SlyB12:Tsx at 3.84 Å and 4.05 Å resolution, respectively (FIG. 20; FIG. 11d). SlyB:Tsx models were built by rigid body docking of SlyB monomers derived from the SlyB₁₁ model and Tsx (PDB ID: 1TLY) into the reconstructed map, followed by manual rebuilding using COOT 44 and PHENIX real-space refinement including NCS averaging and secondary structure restraints 45. See Table 1 for data and model statistics.

Processing small datasets of SlyB pulldown in LB and LB supplemented with 5 mM EDTA. SlyB pulldowns in LB and LB supplemented with 5 mM EDTA were plunged and collected on a 300-kV CRYO ARM™ 300 for small datasets. Motion correction, ctf estimation and particle picking were performed as described above. Multiple 2D classification rounds were deployed to obtain representative 2D classes of SlyB complexes.

SDS PAGE, semi-native PAGE, Blue native PAGE, and immunoblotting. Semi-native PAGE (heat modifiability analysis of OMP folding) was performed using Tris-HCl 4-12% or 10.5% polyacrylamide gradient gels, or precast 4-15% polyacrylamide gradient gels (Biorad), with MES running buffer containing 50 mM MES, 50 mM Tris base, 1 mM EDTA, and 0.1% (w/v) SDS. Samples were mixed in 3:1 ratio with 4×SDS loading dye consisting of 200 mM Tris pH 6.8, 4% SDS (sodium dodecyl sulfate), 0.4% (w/v) bromophenol blue, 40% glycerol, and with/without 200 mM DTT. Mixtures were either heated at 95° C. for 5 minutes (denaturing) or incubated at room temperature (native) before loading on gels. Semi-native PAGE were performed at 150 V for 75 minutes at 4° C. Blue native electrophoresis analysis was carried out on precast 3-12% Bis-Tris gels (Invitrogen) following the manufacturer's instructions. Briefly, 20 μL of each sample was mixed 3:1 with NativePAGE™ sample loading buffer 4× (Invitrogen) and loaded on a NativePAGE™ 3-12%, Bis-Tris gel (Invitrogen). Electrophoresis was run for 90 minutes at 150 volts at 4° C., using NativePAGE™ Running Buffer Kit (Invitrogen). For the two-dimensional SDS-PAGE, whole lanes or individual bands of interest were excised from blue native PAGE, heated in SDS-PAGE sample dye (95° C. for 5 minutes) and applied to the top of a SDS PAGE.

For immunoblotting, proteins from acrylamide gels were transferred in a semi-dry manner (Biorad Trans-Blot Turbo) onto a PVDF membrane (Thermo Fisher) and then blocked in PBST buffer (PBS supplemented with 0.05% Tween-20) supplemented with 4% (w/v) milk powder for 1 hour at RT or overnight at 4° C. while shaking. The membranes were then washed five times with PBST and incubated with primary antibodies for 1 hour at RT (see Table 3 for titers and antibody details). Blots were rinsed with PBST to remove excess unbound antibody and incubated with the corresponding secondary antibody (either horseradish-peroxidase-coupled or Alkaline-phosphatase antibody). Primary and secondary antibodies were diluted in PBST supplemented with 0.4% milk according to the titers reported in Table 3. After final wash with PBST, the membranes were revealed using Clarity ECL Substrate (Bio-Rad) solutions or NBT/BCIP substrate (Roche) and images were acquired and digitized using a Bio-Rad Chemidoc imaging system and ImageLab software.

TABLE 2
Antibodies and titers used in this study.

| Antibody | Description | Dilution | Source |
|---|---|---|---|
| Mouse anti-His | Monoclonal mouse antibody binding His-tag | 1:1000 | ID: MCA1396 Bio-Rad |
| Rabbit anti-SlyB | Polyclonal rabbit antibody binding E. coli SlyB | 1:4000 | This study |
| Rabbit anti-BamA | Polyclonal rabbit antibody binding E. coli BamA | 1:20000 | J. F. Collet |
| Rabbit anti-LptD | Polyclonal rabbit antibody binding E. coli LptD | 1:20000 | J. F. Collet |
| Rabbit anti-OmpC | Polyclonal rabbit antibody binding E. coli OmpC | 1:10000 | ID: ORB28266_100ug |
| | | | Bio-connect |
| Rabbit anti-OmpA | Polyclonal rabbit antibody binding E. coli OmpA | 1:10000 | ID: ORB422682_100ug |
| | | | Bio-connect |
| Rabbit anti-EF-Tu | Monoclonal mouse antibody binding E. coli EF- | 1:2000 | ID: HM6010-20UG |
| | Tu | | Sanbio |
| Goat anti-rabbit | Polyclonal goat antibody fused to horseradish | 1:2000 | ID: A0545-1ML Sigma- |
| HRP | peroxidase (HRP) binding rabbit antibody | | Aldrich |
| Goat anti-mouse | Polyclonal goat antibody fused to HRP binding | 1:2000 | ID: A9917-1ML Sigma- |
| HRP | mouse antibody | | Aldrich |
| Goat anti-rabbit AP | Polyclonal goat antibody fused to alkaline | 1:2000 | ID: A8025-1ML Sigma- |
| | phosphatase (AP) binding rabbit antibody | | Aldrich |

Cell membrane fractionation using sucrose gradient ultracentrifugation. Cell fractionation was performed as described⁷⁶ with some modifications. Briefly, cells were grown at 37° C. in LB until OD600 0.6-0.8, followed by a centrifugation at 5000 g for 15 minutes, and resuspended in lysis buffer, containing 50 mM Tris pH 8, 150 mM NaCl, 0.1 mg·mL⁻¹ lysozyme, 0.05 mg·mL⁻¹ DNAse (Roche), 0.4 mM AEBSF, and 1 μg·mL⁻¹ leupeptin. After incubation of 1 hour at 4° C., cell suspension was passed through a LM10 microfluidizer, at 15.000 psi. The unbroken cells and cell debris were classified and discarded by centrifugation at 5000 g for 15 minutes. The total membrane was collected by 2-step sucrose gradient centrifugation. 40 mL of supernatant was placed on top of 5 ml of 2.02 M sucrose and 14 ml of 0.77 M sucrose gradient, both in 10 mM HEPES pH 7.5. Samples were centrifuged at 40.000 rpm for 3 hours at 4° C. in a 45 Ti Bechman rotor. 6 mL of soluble membrane fraction was carefully collected from the bottom of the centrifuge tubes, then diluted 4 times with 10 mM HEPES pH 8. 0.5 mL of the membrane fraction was then placed on top of a 3-step sucrose gradient (4.2 mL of 2.02 M sucrose, 5 mL of 1.44 M sucrose, 2.8 mL of 0.77 M sucrose, in 10 mM HEPES pH 7.5) in a polyallomer thinwall tube and centrifuged at 35 000 rpm for 18 hours at 4° C. in a SW 41 Ti Beckman rotor. After centrifugation, each polyallomer tube was carefully punched a hole at the bottom to collect different fractions. The amount of SlyB, marker proteins for IM and OM were analysed using specific antibodies. Western blot procedure was described above.

Quantification of cell- and outer membrane vesicles (OMVs) associated BamA. BW25133 and AJ5 cells were grown in specific stress conditions as mentioned (i.e. 1 mM EDTA, 100 μg/ml Bactenecin 2A (Eurogentec) or 0.1 μg/ml LpxC inhibitor PF-04753299 (Sigma-Aldrich)). Cell samples were collected 1 h after stress induction and OD₆₀₀ was normalized to a value of 0.5. To determine cell-associated BamA levels, cells were spun down at 5000 g (10 min), resuspended to an OD₆₀₀ of 10 and 15 μL was used for SDS-PAGE and western blot as previously described with anti-BamA and anti-EF-Tu as primary antibodies. ImageLab software was used to integrate band intensity of obtained western blots. Obtained OM integration values were normalized by EF-Tu levels of the corresponding sample. For OMV preparation growth media were cleared of cells by a 10 min centrifugation at 5000 g, followed by a syringe filtration with 0.22 μm pore size. 40 ml of supernatant was applied on top of an ultracentrifuge tube containing a sucrose gradient consisting of 5 mL of 2.02 M sucrose and 14 mL of 0.77 M sucrose, both in 10 mM HEPES pH 7.5. Samples were centrifuged at 40 000 rpm for 3 hours at 4° C. in a 45 Ti Bechman rotor. OMV-containing fraction was carefully collected from the bottom of the centrifuge tubes. OMV-associated BamA were analysed using western blot as described above for whole cells.

In vitro heat stability assay of purified BamA and purified SlyB-BamA. BamA and SlyB:BamA were purified as described above and stored in SEC buffer (20 mM Tris pH 8, 150 mM NaCl, and 0.03% DDM). Proteins, at concentration of 0.2 mg·mL⁻¹, were aliquoted in PCR tubes, 30 μL each. Tubes were incubated in triplicate at the indicated temperature range for 5 minutes. The BamA folding (i.e. native—folded, or unfolded) status was monitored by band-shift assay on semi-native SDS-PAGE. PAGE were stained with InstantBlue Coomassie Protein Stain (Abcam, ISB1L), imaged with GelDoc Go (Bio-Rad) and band intensities were integrated using ImageLab software (Bio-Rad).

Cellular thermal shift assay (CETSA). 5 ml of LB culture was inoculated with BW25113 or BW25113 ΔslyB. Cells were grown at 37° C. with shaking, for about 2 hours until OD600 reaches 0.4-0.6, followed by a centrifugation at 5000 g for 10 minutes. Cells were washed once in PBS buffer, spun at 5000 g for 10 minutes and resuspended in PBS to OD₆₀₀ of 0.5. Aliquots of 50 μL in PCR tubes were incubated at different temperature from 55 to 100° C., for 10 minutes. Heated samples were subjected to a semi-native SDS-PAGE without boiling in SDS running buffer. BamA was followed using Western blot analysis using anti-BamA antibody and anti-rabbit-HRP antibody. Band intensities were integrated using ImageLab software (Bio-Rad).

OMP quantification. BW25113 and AJ1 were transformed with pET23a_BamA, pET23a_BamA^{R661G/D740G}, pBtuB and pFhuA (Table 2). Cells were grown on non-inducing LB medium (i.e. using leaky expression of T7/tet promotor) at 30° C. for an initial 3 hours, followed by another 6 hours in presence or absence of 5 mM EDTA. Samples were spun down (4000 g, 10 min), resuspended to an OD₆₀₀ of 10 and 15 μL was used for SDS-PAGE and western blot as previously described with anti-His and anti-EF-Tu as primary antibodies. ImageLab software was used to integrate band intensity of obtained western blots. Obtained OM integration values were normalized by EF-Tu levels of the corresponding sample.

Time lapse microscopy imaging. LB-agar slips were prepared as described⁴⁸. In short, an agar slip was made in a gene frame (ABgene 1.7×2.8 cm) on a microscopy slide. Overnight cultures of the different E. coli strains were diluted to an OD₆₀₀ of 0.05 and 3 μL was transferred to the LB-agar strip. The slides were imaged in a preheated incubating chamber (37° C.). Images were taken in phase contrast mode on a Leica DMi8 with a 100× objective. Images were taken every 5 minutes and Leica adaptive focus control was used for autofocusing before each image.

Macrophage survival assays. J774A.1 murine macrophages (ATCC) were cultured at 37° C. under 5% CO2 in DMEM (GIBCO) supplemented with 10% fetal bovine serum (Lonza). For the infection assay, the macrophages at 80-90% confluency were harvested and 200 μL was transferred to each well of a 96-well plate at 2.5×10⁵ cells·mL⁻¹ for overnight growth and adhesion to the plate. The next day, the tested bacterial strains were collected in exponential growth phase (OD600 0.6-1.2) and diluted in DMEM to 5×10⁶ cells·mL⁻¹ to perform infection experiments with a multiplicity of infection of 20:1. Bacteria and cells were centrifuged at 1000 g for 10 min and incubated at 37° C., 5% CO2. 30 minutes after the beginning of infection, the culture medium was replaced with DMEM supplemented with 100 μg·mL⁻¹ of gentamicin to kill the remaining extracellular bacteria. One hour later, culture medium was changed with fresh DMEM supplemented with 10 μL·ml⁻¹ of gentamicin and left for 22.5 h at 37° C., 5% CO₂. To quantify intracellular bacteria, the macrophages were washed twice with PBS and lysed in PBS with 0.2% Triton X-100 for 10 min at 37° C. Dilutions were plated onto LB agar, incubated at 37° C. to determine colony-forming units (CFU). Experiments were performed in 12 biological repeats, and statistical analysis performed by an unpaired T test with a lack of difference between WT and the isogenic ΔslyB mutants as null hypothesis.

Mass spectrometry fingerprint. The protein bands excised from the gel were crushed in small pieces of about 1 mm³. The gel pieces were washed with 25 mM NH₄HCO₃ and 50% CH₃CN/25 mM NH₄HCO₃ and then dried in a vacuum centrifuge. The gel pieces were swollen in an ice-cold bath in 10 μl of a digestion buffer containing 25 mM NH₄HCO₃, and 10 ng·μL⁻¹ of sequencing grade trypsin/Lys-C (Promega). The digestion was carried out overnight at 37° C. The supernatants were collected and the peptides remaining in the gel pieces were extracted sequentially by 25 mM NH₄HCO₃, 50% CH₃CN/25 mM NH₄HCO₃ and 50% CH₃CN/5% HCOOH. The supernatants and the lavages were pooled and dried in a vacuum centrifuge. Before analysis by mass spectrometry, the samples were desalted on ZipTip C18 (Millipore) and eluted in 50% acetonitrile/1% formic acid (v/v). The samples were loaded into a nanoflow capillary (Proxeon) and ESI mass spectra were acquired on a quadrupole time-of-flight instrument (Q-Tof Ultima—Micromass/Waters) operating in the positive ion mode, equipped with a Z-spray nanoelectrospray source. Data acquisition was performed using a MassLynx 4.1 system. The sequence of the peptides was determined by tandem mass spectrometry (MS/MS). After processing of the MS/MS data by the maximum entropy data enhancement program MaxEnt 3, the amino acid sequence was semi-automatically deduced using the peptide sequencing program PepSeq (Waters). Based on the peptide sequences, the proteins were identified using the MASCOT Sequence Query.

### Quantitative Mass Spectrometry of S/B Interactome and OM Proteome.

Sample preparation. Samples for SlyB interactome analyses were prepared using the 2-step His-tag pulldown protocol described above, using E. coli AJ1 attTN7::slyB and AJ1 attTN7::slyB^{TEV_His} grown in LB or LB supplemented with 5 mM EDTA. The SlyB interactome in LB or LB+EDTA conditions was determined by differential shotgun LC-MS/MS analysis of both SlyB^{TEV_His} pulldowns (LB and LB+EDTA), with the equivalent 2-step His-tag pulldown of AJ1 attTN7::slyB (i.e. non-tagged SlyB) as reference sample for non-specific contaminants. Samples for OM proteomics analyses were prepared as follows. 3 ml overnight culture of BW25113 or BW25113 ΔslyB were used to inoculate 300 mL LB or LB+EDTA (2 mM). Cells were grown until and OD600 of 0.4 and then pelleted at 5000 g for 15 minutes before resuspension in lysis buffer, containing 50 mM Tris pH 8, 300 mM NaCl, 0.1 mg·mL⁻¹ lysozyme, 0.05 mg·mL⁻¹ DNA and 5 mM MgCl₂, 1 μg·mL⁻¹ Leupeptin and 0.1 mg·mL⁻¹ AEBSF, and lysed by sonication. Unbroken cells and cell debris were discarded by centrifugated at 5000 g for 15 minutes. The total membrane was pelleted using an ultracentrifugation at 100.000 g for 45 minutes. These membrane samples were washed once in 50 mM Tris pH 8, 300 mM NaCl buffer and another ultracentrifugation round. The final membrane samples were then solubilized in 50 mM Tris pH8, 300 mM NaCl, 1.5% DDM, 0.5% LDAO, 1 μg·mL⁻¹ Leupeptin, 0.1 mg·mL⁻¹ AEBSF for 2 h at 4° C. The supernatant was cleared by ultracentrifugation at 100.000 g and sent for mass spectrometry. All samples were grown and processed in biological triplicate. MS proteome analysis were performed at the VIB Proteomics Core (https://cmb.vib.be/labs/vib-proteomics-core #.

MS analysis of the SlyB interactome. Per sample, 600 μg eluted proteins were reduced with 5 mM dithiothreitol (DTT) (SigmaAldrich) for 30 min at 55° C. in 8M urea buffer. Alkylation was performed by the addition of 10 mM iodoacetamide (Sigma-Aldrich) for 15 min at room temperature in the dark. The samples were diluted with 20 mM N-(2-hydroxyethyl)piperazine-N′-ethanesulfonic acid (HEPES), pH 8.0, to a urea concentration of 4 M, and digested for 4 hours with 1 μl endolysC at 370 followed by a second dilution with 20 mM HEPES to 2 M urea and overnight digestion with 1 μg of trypsin (V5111, Promega) (1/100, w/w) at 37° C. The digested peptides were cleaned up with Phoenix clean-up cartridges (PreOmics) according to the manufacturer's protocol and purified peptides were dried completely in a rotary evaporator. Peptides were re-dissolved in 80 μl loading solvent A (0.1% TFA in water/ACN (98:2, v/v)) of which 2 μl was injected for LC-MS/MS analysis on an Ultimate 3000 RSLC nano LC (Thermo Fisher Scientific, Bremen, Germany) in-line connected to a Q Exactive mass spectrometer (Thermo Fisher Scientific). Peptides were first loaded on a trapping column made in-house (100 μm internal diameter (I.D.)×20 mm, 5 μm beads C18 Reprosil-HD, Dr. Maisch, Ammerbuch-Entringen, Germany) and after flushing from the trapping column the peptides were separated on a 50 cm μPAC™ column with C18-endcapped functionality (Pharmafluidics, Belgium) kept at a constant temperature of 35° C. Peptides were eluted by a stepped gradient from 98% solvent A′ (0.1% formic acid in water) to 30% solvent B′ (0.1% formic acid in water/acetonitrile, 20/80 (v/v)) in 75 min, and up to 50% solvent B′ in 25 min at a flow rate of 300 nL/min, followed by a 5 min wash reaching 95% solvent B′. The mass spectrometer was operated in data-dependent, positive ionization mode, automatically switching between MS and MS/MS acquisition for the 5 most abundant peaks in a given MS spectrum. The source voltage was 3.5 kV, and the capillary temperature was 275° C. One MS1 scan (m/z 400-2,000, AGC target 3×E6 ions, maximum ion injection time 80 ms), acquired at a resolution of 70,000 (at 200 m/z), was followed by up to 5 tandem MS scans (resolution 17,500 at 200 m/z) of the most intense ions fulfilling predefined selection criteria (AGC target 50.000 ions, maximum ion injection time 200 ms, isolation window 2 Da, fixed first mass 140 m/z, spectrum data type: centroid, intensity threshold 1.3×E4, exclusion of unassigned, 1, 5-8, >8 positively charged precursors, peptide match preferred, exclude isotopes on, dynamic exclusion time 12 s). The HCD collision energy was set to 25% Normalized Collision Energy and the polydimethylcyclosiloxane background ion at 445.120025 Da was used for internal calibration (lock mass). QCloud was used to control instrument longitudinal performance during the project 4. LC-MS/MS runs of all 12 samples were searched together using the MaxQuant algorithm (version 1.6.9.0) with mainly default search settings, including a false discovery rate set at 1% on peptide and protein level and carbamidomethylation on cysteine residues as a fixed modification, oxidation on methionines and acetylation on the N-terminus as variable modifications. Spectra were searched against the E. coli (strain K12) protein sequences in the Uniprot database, containing 4,519 sequences (https://www.uniprot.org/, release version of June 2019, taxid 83333), supplemented with the SlyB_TEV_His sequence. A total of 211,817 peptide-to-spectrum matches (PSMs) were performed, resulting in 11,026 identified unique peptides, corresponding to 1,516 identified proteins, of which 1,136 protein groups were reliably quantified (=protein groups with at least 3 valid LFQ intensity values in one of the experimental conditions. PCA analysis showed clustering of the biological triplicates and separation of the four sample preparation conditions (i.e. AJ1::slyB LB, AJ1::slyB LB+EDTA, AJ1::slyB^{TEV_His} LB and AJ1::slyB^{TEV_HIS} LB+EDTA). To compare protein intensities in the AJ1::slyB^{TEV_His} LB and AJ1::slyB LB samples, a t-test was performed for each protein. Statistical significance for differential regulation was set at FDR<0.01 and s0=1. Results are shown in FIG. 2. When applying a fold change cut-off of 10 and a p-value of 0.01, 26 proteins were significantly pulled down as SlyB interactors under LB conditions. Applying an equivalent procedure and significance cut-off of the AJ1::slyB^{TEV_His} LB+EDTA and AJ1::slyB LB+EDTA samples, 110 proteins were identified as significantly pulled down as SlyB interactors under LB+EDTA conditions.

MS analysis of the OM proteome. Per sample 600 μg of protein per sample were reduced with 5 mM dithiothreitol (DTT) (SigmaAldrich) for 30 min at 55° C. in 8 M urea buffer. Alkylation, trypsin digestion and LC MS/MS acquisition and analysis were performed as described above for the SlyB interactome. A total of 125,234 peptide-to-spectrum matches (PSMs) were performed, resulting in 15,528 identified unique peptides, corresponding to 1,665 identified proteins, of which 1,204 protein groups were reliably quantified (=protein groups with at least 3 valid LFQ intensity values in one of the experimental conditions. PCA analysis showed clustering of the biological triplicates and separation of the four sample preparation conditions (i.e. BW25113 LB, BW25113 LB+EDTA, BW25113 ΔslyB LB and BW25113 ΔslyB LB+EDTA). To compare protein intensities in the LB and LB+EDTA conditions for the BW25113 and BW25113 ΔslyB, a t-test was performed for each. Statistical significance for differential regulation was set at FDR<0.01 and s0=1. Results are shown in FIG. 18d.

### Immunoassay Procedure.

Antigen preparation. The purified SlyB OMD complexes (or SlyB nanodiscs) used for immunization were produced in EDTA-stressed outer membrane of E. coli AJ1 attTN7::slyB^{TEV_His} (see above), following detergent-extraction, and purified by 2-step IMAC Ni-affinity chromatography and size exclusion chromatography as described (see above: ‘Pulldown of SlyB complexes’). Purified SlyB:BamA nanodiscs were detergent extracted from EDTA-stressed E. coli AJ1 attTN7::slyB^{TEV_His} pBamA and purified by 2-step IMAC Ni-affinity chromatography and size exclusion chromatography as described (see above: ‘Production and purification of SlyB:OMP complexes’). Purified SlyB OMDs and SlyB:BamA nanodisc complexes were formulated in 20 mM Tris pH 8, 150 mM NaCl, 0.03% DDM. Pure protein antigens were prepared with pBamA, pLptDE, pBtuB or pFhuA encoding N-terminally His-TEV tagged version of E. coli BamA (Uniprot: POA940), LptDE (Uniprot: P31554, POADC1), BtuB (Uniprot: P06129) or FhuA (Uniprot: P06971) in a modified pASK-Iba12 vector (see above). Proteins were detergent extracted from the respective cells, and purified by 2-step Ni-IMAC affinity purification, and formulated in 20 mM Tris pH 8, 150 mM NaCl, 0.03% DDM.

The presence and strength of the humoral immune response in animals immunized with SlyB nanodiscs (SlyB OMDs or SlyB:BamA) or control antigens (BamA, LptDE, BtuB or FhuA) was determined using a microfiltration dot blot procedure. In brief, 200 ng of the respective antigen was immobilized on a nitrocellulose membrane (with or without prior 10 min 95° C. heat treatment), blocked with LiCor Intercept Blocking TBS, and exposed to 100 all per well of the respective mouse immune sera at indicated dilution (i.e. from 1:100 to 1:102400 in TBS). Washed membranes (2× in TTBS), were exposed to 100 μl per well of IRDye 800 CW goat anti-mouse, used at 1:20.000 as the secondary antibody (recognizing mouse IgG1, IgG2a, IgG2b, and IgG3, IgM and IgA), and again washed 2× with TTBS prior to readout of the 800 nm epifluorescence signal on a LI-COR Odyssey M imager. Integrated fluorescence intensities were plotted against serum dilution to determine immune titers (FIGS. 21-23). Data represent averages of two biological replicates. To determine the immune titer for linear versus non-linear epitopes, antigens were coated with or without heat denaturation by a 10 min 95° C. heat treatment prior to coating. To evaluate the immune titer for surface localized antigens in whole cell E. coli, an equivalent microfiltration dot blot procedure was performed, with the exception that rather than with the purified antigens, the nitrocellulose membranes were coated with 50 μl per well of life E. coli cells at OD₆₀₀ of 0.01 in TBS, using E. coli strain MG1655 and its deep rough mutant ΔrfaD to evaluate the influence of the LPS inner core to antibody binding.

### REFERENCES

1 Nikaido, H. Molecular basis of bacterial outer membrane permeability revisited. Microbiol Mol Biol Rev 67, 593-656 (2003).
2 Whitfield, C. & Trent, M. S. Biosynthesis and export of bacterial lipopolysaccharides. Annu Rev Biochem 83, 99-128 (2014).
3 Rojas, E. R. et al. The outer membrane is an essential load-bearing element in Gram-negative bacteria. Nature 559, 617-621 (2018).
4 Lundstedt, E., Kahne, D. & Ruiz, N. Assembly and Maintenance of Lipids at the Bacterial Outer Membrane. Chem Rev (2020).
5 Guest, R. L., Rutherford, S. T. & Silhavy, T. J. Border Control: Regulating LPS Biogenesis. Trends Microbiol (2020).
6 Simpson, B. W. & Trent, M. S. Pushing the envelope: LPS modifications and their consequences. Nat Rev Microbiol 17, 403-416 (2019).
7 Sutterlin, H. A. et al. Disruption of lipid homeostasis in the Gram-negative cell envelope activates a novel cell death pathway. Proc Natl Acad Sci USA 113, E1565-1574 (2016).
8 Hagan, C. L., Silhavy, T. J. & Kahne, D. beta-Barrel membrane protein assembly by the Bam complex. Annu Rev Biochem 80, 189-210 (2011).
9 Noinaj, N., Gumbart, J. C. & Buchanan, S. K. The beta-barrel assembly machinery in motion. Nat Rev Microbiol 15, 197-204 (2017).
10 Okuda, S. & Tokuda, H. Lipoprotein sorting in bacteria. Annu Rev Microbiol 65, 239-259 (2011).
11 De Geyter, J. et al. Protein folding in the cell envelope of Escherichia coli. Nat Microbiol 1, 16107 (2016).
12 Perez, J. C. et al. Evolution of a bacterial regulon controlling virulence and Mg(2+) homeostasis. PLoS Genet 5, e1000428 (2009).
13 Monsieurs, P. et al. Comparison of the PhoPQ regulon in Escherichia coli and Salmonella typhimurium. J Mol Evol 60, 462-474 (2005).
14 Groisman, E. A. The pleiotropic two-component regulatory system PhoP-PhoQ. J Bacteriol 183, 1835-1842 (2001).
15 Richards, S. M., Strandberg, K. L., Conroy, M. & Gunn, J. S. Cationic antimicrobial peptides serve as activation signals for the Salmonella Typhimurium PhoPQ and PmrAB regulons in vitro and in vivo. Front Cell Infect Microbiol 2, 102 (2012).
16 Murata, T., Tseng, W., Guina, T., Miller, S. I. & Nikaido, H. PhoPQ-mediated regulation produces a more robust permeability barrier in the outer membrane of Salmonella enterica serovar typhimurium. J Bacteriol 189, 7213-7222 (2007).
17 Deich, R. A. et al. Antigenic conservation of the 15,000-dalton outer membrane lipoprotein PCP of Haemophilus influenzae and biologic activity of anti-PCP antisera. Infect Immun 58, 3388-3393 (1990).
18 Plesa, M., Hernalsteens, J. P., Vandenbussche, G., Ruysschaert, J. M. & Cornelis, P. The SlyB outer membrane lipoprotein of Burkholderia multivorans contributes to membrane integrity. Res Microbiol 157, 582-592 (2006).
19 Firoved, A. M. & Deretic, V. Microarray analysis of global gene expression in mucoid Pseudomonas aeruginosa. J Bacteriol 185, 1071-1081 (2003).
20 Lima, S., Guo, M. S., Chaba, R., Gross, C. A. & Sauer, R. T. Dual molecular signals mediate the bacterial response to outer-membrane stress. Science 340, 837-841 (2013).
21 Wu, M. & Hancock, R. E. Interaction of the cyclic antimicrobial cationic peptide bactenecin with the outer and cytoplasmic membrane. J Biol Chem 274, 29-35 (1999).
22 Cunrath, O. & Bumann, D. Host resistance factor SLC11A1 restricts Salmonella growth through magnesium deprivation. Science 366, 995-999 (2019).
23 Thompson, J. A., Liu, M., Helaine, S. & Holden, D. W. Contribution of the PhoP/Q regulon to survival and replication of Salmonella enterica serovar Typhimurium in macrophages. Microbiology (Reading) 157, 2084-2093 (2011).
24 Li, G. W., Burkhardt, D., Gross, C. & Weissman, J. S. Quantifying absolute protein synthesis rates reveals principles underlying allocation of cellular resources. Cell 157, 624-635 (2014).
25 Kim, S. et al. Transmembrane glycine zippers: physiological and pathological roles in membrane proteins. Proc Natl Acad Sci USA 102, 14278-14283 (2005).
26 Xiao, L. et al. Structures of the beta-barrel assembly machine recognizing outer membrane protein substrates. FASEB J 35, e21207 (2021).
27 Dwyer, R. S., Ricci, D. P., Colwell, L. J., Silhavy, T. J. & Wingreen, N. S. Predicting functionally informative mutations in Escherichia coli BamA using evolutionary covariance analysis. Genetics 195, 443-455 (2013).
28 Koski, P. & Vaara, M. Polyamines as constituents of the outer membranes of Escherichia coli and Salmonella typhimurium. J Bacteriol 173, 3695-3699 (1991).
29 Takeuchi, Y. & Nikaido, H. Persistence of segregated phospholipid domains in phospholipid-lipopolysaccharide mixed bilayers: studies with spin-labeled phospholipids. Biochemistry 20, 523-529 (1981).
30 Wu, T. et al. Identification of a protein complex that assembles lipopolysaccharide in the outer membrane of Escherichia coli. Proc Natl Acad Sci USA 103, 11754-11759 (2006).
31 Ruiz, N., Wu, T., Kahne, D. & Silhavy, T. J. Probing the barrier function of the outer membrane with chemical conditionality. ACS Chem Biol 1, 385-395 (2006).
32 May, K. L. & Silhavy, T. J. The Escherichia coli Phospholipase PldA Regulates Outer Membrane Homeostasis via Lipid Signaling. mBio 9 (2018).
33 Katz, C. & Ron, E. Z. Dual role of FtsH in regulating lipopolysaccharide biosynthesis in Escherichia coli. J Bacteriol 190, 7117-7122 (2008).
34 Akiyama, Y. Quality control of cytoplasmic membrane proteins in Escherichia coli. J Biochem 146, 449-454 (2009).
35 Datsenko, K. A. & Wanner, B. L. One-step inactivation of chromosomal genes in Escherichia coli K-12 using PCR products. Proc Natl Acad Sci USA 97, 6640-6645 (2000).
36 Thomason, L. C., Costantino, N. & Court, D. L. E. coli genome manipulation by P1 transduction. Curr Protoc Mol Biol Chapter 1, Unit 1 17 (2007).
37 Schmieger, H. Phage P22-mutants with increased or decreased transduction abilities. Mol Gen Genet 119, 75-88 (1972).
38 McKenzie, G. J. & Craig, N. L. Fast, easy and efficient: site-specific insertion of transgenes into enterobacterial chromosomes using Tn7 without need for selection of the insertion event. BMC Microbiol 6, 39 (2006).
39 Zheng, S. Q. et al. MotionCor2: anisotropic correction of beam-induced motion for improved cryo-electron microscopy. Nat Methods 14, 331-332 (2017).
40 Rohou, A. & Grigorieff, N. CTFFIND4: Fast and accurate defocus estimation from electron micrographs. J Struct Biol 192, 216-221 (2015).
41 Wagner, T. et al. SPHIRE-crYOLO is a fast and accurate fully automated particle picker for cryo-EM. Commun Biol 2, 218 (2019).
42 Punjani, A., Rubinstein, J. L., Fleet, D. J. & Brubaker, M. A. cryoSPARC: algorithms for rapid unsupervised cryo-EM structure determination. Nat Methods 14, 290-296 (2017).
43 Terwilliger, T. C., Ludtke, S. J., Read, R. J., Adams, P. D. & Afonine, P. V. Improvement of cryo-EM maps by density modification. Nat Methods 17, 923-927 (2020).
44 Emsley, P. & Cowtan, K. Coot: model-building tools for molecular graphics. Acta Crystallogr D Biol Crystallogr 60, 2126-2132 (2004). Afonine, P. V. et al. Real-space refinement in PHENIX for cryo-EM and crystallography. Acta Crystallogr D Struct Biol 74, 531-544 (2018).
46 Moriarty, N. W., Grosse-Kunstleve, R. W. & Adams, P. D. electronic Ligand Builder and Optimization Workbench (eLBOW): a tool for ligand coordinate and restraint generation. Acta Crystallogr D Biol Crystallogr 65, 1074-1080 (2009).
47 Pettersen, E. F. et al. UCSF Chimera—a visualization system for exploratory research and analysis. J Comput Chem 25, 1605-1612 (2004).
48 de Jong, I. G., Beilharz, K., Kuipers, O. P. & Veening, J. W. Live Cell Imaging of Bacillus subtilis and Streptococcus pneumoniae using Automated Time-lapse Microscopy. J Vis Exp (2011).
49 Chiva, C. et al. QCloud: A cloud-based quality control system for mass spectrometry-based proteomics laboratories. PLoS One 13, e0189209 (2018).
50 Zivanov, J. et al. New tools for automated high-resolution cryo-EM structure determination in RELION-3. Elife 7 (2018).
51 Sartorio M G, Pardue E J, Feldman M F, Haurat M F. Bacterial Outer Membrane Vesicles: From Discovery to Applications. Annu Rev Microbiol. 2021 Aug. 5. doi: 10.1146/annurev-micro-052821-031444. Epub ahead of print. PMID: 34351789.
52 Holst J, Oster P, Arnold R, Tatley M V, Nmss L M, Aaberge I S, Galloway Y, McNicholas A, O'Hallahan J, Rosenqvist E, Black S. Vaccines against meningococcal serogroup B disease containing outer membrane vesicles (OMV): lessons from past programs and implications for the future. Hum Vaccin Immunother. 2013 June; 9(6):1241-53. doi: 10.4161/hv.24129. Epub 2013 Mar. 7. PMID: 23857274; PMCID: PMC3901813.
53 Needham B D, Carroll S M, Giles D K, Georgiou G, Whiteley M, Trent M S. Modulating the innate immune response by combinatorial engineering of endotoxin. Proc Natl Acad Sci USA. 2013 Jan. 22; 110(4):1464-9. doi: 10.1073/pnas.1218080110. Epub 2013 Jan. 7. PMID: 23297218; PMCID: PMC3557076
54 Vinés E D, Marolda C L, Balachandran A, Valvano M A. Defective O-antigen polymerization in tolA and pal mutants of Escherichia coli in response to extracytoplasmic stress. J Bacteriol. 2005 May; 187(10):3359-68. doi: 10.1128/JB.187.10.3359-3368.2005. PMID: 15866920; PMCID: PMC1112028.
55 Micoli F, MacLennan C A. Outer membrane vesicle vaccines. Semin Immunol. 2020 August; 50:101433. doi: 10.1016/j.smim.2020.101433. Epub 2020 Dec. 9. PMID: 33309166.
56 Galen J E, Zhao L, Chinchilla M, Wang J Y, Pasetti M F, Green J, Levine M M. Adaptation of the endogenous Salmonella enterica serovar Typhi clyA-encoded hemolysin for antigen export enhances the immunogenicity of anthrax protective antigen domain 4 expressed by the attenuated live-vector vaccine strain CVD 908-htrA. Infect Immun. 2004 December; 72(12):7096-106. doi: 10.1128/IAI.72.12.7096-7106.2004. PMID: 15557633; PMCID: PMC529119.
57 Yang Z, Hua L, Yang M, Liu S Q, Shen J, Li W, Long Q, Bai H, Yang X, Ren Z, Zheng X, Sun W, Ye C, Li D, Zheng P, He J, Chen Y, Huang W, Peng X, Ma Y. RBD-Modified Bacterial Vesicles Elicited Potential Protective Immunity against SARS-CoV-2. Nano Lett. 2021 Jul. 28; 21(14):5920-5930. doi: 10.1021/acs.nanolett.1c00800. Epub 2021 Jul. 19. PMID: 34279108; PMCID: PMC8315139
58 Baba, T., T. Ara, M. Hasegawa, Y. Takai, Y. Okumura, M. Baba, K. A. Datsenko, M. Tomita, B. L. Wanner and H. Mori (2006). “Construction of Escherichia coli K-12 in-frame, single-gene knockout mutants: the Keio collection.” Mol Syst Biol 2: 2006 0008.
59 Cho, S. H., J. Szewczyk, C. Pesavento, M. Zietek, M. Banzhaf, P. Roszczenko, A. Asmar, G. Laloux, A. K. Hov, P. Leverrier, C. Van der Henst, D. Vertommen, A. Typas and J. F. Collet (2014). “Detecting envelope stress by monitoring beta-barrel assembly.” Cell 159(7): 1652-1664.
60 Datsenko, K. A. and B. L. Wanner (2000). “One-step inactivation of chromosomal genes in Escherichia coli K-12 using PCR products.” Proc Natl Acad Sci USA 97(12): 6640-6645.
61 Desmidt, M., R. Ducatelle, J. Mast, B. M. Goddeeris, B. Kaspers and F. Haesebrouck (1998). “Role of the humoral immune system in Salmonella enteritidis phage type four infection in chickens.” Vet Immunol Immunopathol 63(4): 355-367.
62 Gulig, P. A. and R. Curtiss, 3rd (1987). “Plasmid-associated virulence of Salmonella typhimurium.” Infect Immun 55(12): 2891-2901.
63 Guzman, L. M., D. Belin, M. J. Carson and J. Beckwith (1995). “Tight regulation, modulation, and high-level expression by vectors containing the arabinose PBAD promoter.” J Bacteriol 177(14): 4121-4130.
64 Lilleengen, K. (1948). “Typing Salmonella typhimurium by means of bacteriophage.” Acta Path. Microbiol. Scand.
65 McKenzie, G. J. and N. L. Craig (2006). “Fast, easy and efficient: site-specific insertion of transgenes into enterobacterial chromosomes using Tn7 without need for selection of the insertion event.” BMC Microbiol 6: 39.
66 Mulvey, M. A., J. D. Schilling and S. J. Hultgren (2001). “Establishment of a persistent Escherichia coli reservoir during the acute phase of a bladder infection.” Infect Immun 69(7): 4572-4579.
67 Mandi, Y., Farkas, G., Ocsovszky, I. et al. Inhibition of tumor necrosis factor production and ICAM-1 expression by pentoxifylline: beneficial effects in sepsis syndrome. Res. Exp. Med. 195, 297-307 (1995). https://doi.org/10.1007/BF02578000
68 Ziegler-Heitbrock H W, Thiel E, Futterer A, Herzog V, Wirtz A, Riethmuller G. Establishment of a human cell line (Mono Mac 6) with characteristics of mature monocytes. Int J Cancer. 1988 Mar. 15; 41(3):456-61. doi: 10.1002/ijc.2910410324. PMID: 3162233.
69 Gu, Y., Li, H., Dong, H., Zeng, Y., Zhang, Z., Paterson, N. G., Stansfeld, P. J., Wang, Z., Zhang, Y., Wang, W., Dong, C. (2016). Structural basis of outer membrane protein insertion by the BAM complex. Nature 531: 47-52. DOI: 10.1038/nature17199.
70 Armenteros et al. (2019). SignalP 5.0 improves signal peptide predictions using deep neural networks. Nature Biotechnology, 37, 420-423, doi:10.1038/s41587-019-0036-z.
71 Nielsen et al. (1997). Identification of prokaryotic and eukaryotic signal peptides and prediction of their cleavage sites. Protein Engineering, 10:1-6.
72 Brown, M. F. et al. Potent inhibitors of LpxC for the treatment of Gram-negative infections. J Med Chem 55, 914-923 (2012).
73 Cascales, E., Bernadac, A., Gavioli, M., Lazzaroni, J. C. & Lloubes, R. Pal lipoprotein of Escherichia coli plays a major role in outer membrane integrity. J Bacteriol 184, 754-759 (2002).
74 Mathelie-Guinlet, M., Asmar, A. T., Collet, J. F. & Dufrene, Y. F. Lipoprotein Lpp regulates the mechanical properties of the E. coli cell envelope. Nat Commun 11, 1789 (2020).
75 Zivanov, J. et al. New tools for automated high-resolution cryo-EM structure determination in RELION-3. Elife 7 (2018).
76 El Rayes, J., Rodriguez-Alonso, R. & Collet, J. F. Lipoproteins in Gram-negative bacteria: new insights into their biogenesis, subcellular targeting and functional roles. Curr Opin Microbiol 61, 25-34 (2021).
77 Sun, J., Rutherford, S. T., Silhavy, T. J. & Huang, K. C. Physical properties of the bacterial outer membrane. Nat Rev Microbiol 20, 236-248 (2022).
78 Herrera, C. M., Hankins, J. V. & Trent, M. S. Activation of PmrA inhibits LpxT-dependent phosphorylation of lipid A promoting resistance to antimicrobial peptides. Mol Microbiol 76, 1444-1460 (2010).
79 Wang, C. H., Siu, L. K., Chang, F. Y., Chiu, S. K. & Lin, J. C. A Resistance Mechanism in Non-mcr Colistin-Resistant Escherichia coli in Taiwan: R81H Substitution in PmrA Is an Independent Factor Contributing to Colistin Resistance. Microbiol Spectr 9, e0002221 (2021).
80 Darfeuille-Michaud, A., Neut, C., Barnich, N., Lederman, E., Di Martino, P., Desreumaux, P., Gambiez, L., Joly, B., Cortot, A., and Colombel, J. F. (1998). Presence of adherent Escherichia coli strains in ileal mucosa of patients with Crohn's disease. Gastroenterology 115, 1405-1413.

1. An isolated SlyB nanodisc comprising:
a discoidal SlyB oligomer for forming a membrane scaffolding protein belt, comprising two or more SlyB proteins,
a lipid bilayer nanodomain comprising one or more phospholipid layers, which is enclosed in the lumen of the discoidal SlyB oligomer, and
a saccharolipid moiety surrounding the SlyB oligomer.

2. The SlyB nanodisc of claim 1, wherein the SlyB proteins comprise any one of SEQ ID NOs: 57-84 or a bacterial homologue with at least 80% identity of any one thereof.

3. The SlyB nanodisc of claim 1, wherein the isolated SlyB nanodisc has a diameter below 40 nm.

4. The SlyB nanodisc of claim 1, wherein the saccharolipid moiety comprises a lipid A, a lipooligosaccharide (LOS), a lipopolysaccharide (LPS), or a modified LPS molecule.

5. The SlyB nanodisc of claim 1, further comprising at least one macromolecule within the lipid bilayer nanodomain of the SlyB nanodisc.

6. The SlyB nanodisc of claim 5, wherein the at least one macromolecule is a membrane protein, outer membrane protein, outer membrane lipoprotein, transmembrane protein, or β-barrel-containing membrane protein.

7. The SlyB nanodisc of claim 5, wherein the at least one macromolecule is a molecule present in a virus, a bacterium, a fungus, a protozoan, a parasite, a human neoplastic cell or an animal neoplastic, tumor or cancer cell.

8. The SlyB nanodisc of claim 5, wherein the SlyB nanodisc-macromolecular complex has a molecular weight of 150 to 1000 kDa.

9. The SlyB nanodisc of claim 1, wherein the nanodisc is immunogenic.

10. The SlyB nanodisc of claim 1, wherein the nanodisc is comprised in a host cell.

11. A method of producing a SlyB nanodisc, the method comprising:
a. Inducing SlyB expression in a host cell,
b. Extracting one or more SlyB nanodisc particles from the host cell culture, and optionally,
c. Isolating the SlyB nanodisc particles with a diameter below 40 nm.

12. The method according to claim 11, wherein SlyB expression in step a. is induced in the host cell through:
overexpression of recombinant SlyB from a chimeric gene comprising a heterologous inducible promoter operably linked to a nucleic acid encoding a SlyB protein, or
lowering the pH of the cell culture to pH 5 or lower, addition of a cationic antimicrobial peptide to the cell culture, addition of a LPS synthesis inhibitor, depleting divalent cations from the cell culture, addition of a SlyB agonist to said cell culture, or
recombinantly overexpressing a membrane protein in the host cell.

13. The method according to claim 11, wherein the host cell is deficient in endogenous SlyB expression, and recombinantly expressing a SlyB protein comprising SEQ ID NO: 57-84 or a bacterial homologue with at least 80% identity of any one thereof.

14. The method according to claim 11, further comprising the step of:
d. mixing the isolated SlyB nanodisc particles with a macromolecule to allow encapsulation of the macromolecule within the SlyB nanodisc.

15. The method according to claim 11, wherein SlyB expression is induced through recombinant overexpression of a heterologous membrane protein in the host cell, and producing self-adjuvanting SlyB nanodisc particles comprising the heterologous membrane protein encapsulated in the SlyB nanodisc particles.

16. (canceled)

17. A method of immunizing a subject, the method comprising administering to the subject the SlyB nanodisc of claim 1.

18. The method according to claim 17, wherein the SlyB nanodisc elicits an immune response against the macromolecule present in the SlyB nanodisc.

19. (canceled)

20. (canceled)

21. (canceled)

22. The SlyB nanodisc of claim 1, wherein the isolated SlyB nanodisc has a diameter below 20 nm.

23. The SlyB nanodisc of claim 1, wherein the saccharolipid moiety comprises a lipid A, a lipooligosaccharide (LOS), a lipopolysaccharide (LPS), or a modified LPS molecule, in a ratio of 1:1 with SlyB protomers.

24. The SlyB nanodisc of claim 10 wherein the host cell is deficient in endogenous SlyB expression.

25. The method according to claim 17, wherein the SlyB nanodisc comprises at least one macromolecule within the lipid bilayer nanodomain of the SlyB nanodisc.
